(19) United States
(12) Patent Application Publication
(10) Pub. No.: US 2016/0367188 A1
(43) Pub. Date: Dec. 22, 2016

(54) ORAL SENSOR ALERTING AND COMMUNICATION SYSTEM AND DEVELOPERS' TOOL KIT

(71) Applicants: Bela Malik, Corpus Christi, TX (US); Michael M. Saigh, Clayton, MO (US)

(72) Inventors: Bela Malik, Corpus Christi, TX (US); Michael M. Saigh, Clayton, MO (US)

(21) Appl. No.: 14/850,713

(22) Filed: Sep. 10, 2015

Related U.S. Application Data
(60) Provisional application No. 62/180,841, filed on Jun. 17, 2015.

(51) Int. Cl. A61B 5/00; A61B 5/145; A61J 17/00; A61B 5/08; A61B 5/01; A61B 5/07; A61B 5/024

(52) U.S. Cl. CPC A61B 5/682; A61B 5/076; A61B 5/14542; A61B 5/024; A61B 5/0816; A61B 5/01; A61B 5/4839; A61J 17/003; A61J 17/006; A61B 5/4866; A61B 2503/40; A61B 2562/028; A61B 2562/0285; A61B 2503/04

(57) ABSTRACT

A developers' tool kit including devices and components configured to be inserted or imbedded in an oral cavity or integrated body sensor of an animal or human. The device includes a receptacle for one or more sensors which is further configured to interface a plurality of one or more customizable functions and applications. The functions including, but not limited to, auxiliary body biosensors, data collections, alerting, tracking, reporting, communication network, preset biometric range, preventive-diagnostics to enhance health, and optimize athletic performance. The system includes technology-mining, data-mining, kinematics, integration, multi-media, reporting, and other platforms, analytics and diagnostics to accurately determine health and performance referred to as Oral Sensor Alert and Communication (OSAC). The invention provides innovative information systems, methods and diagnostic tool kits and platforms involving the aforedescribed devices from information available from oral biomarkers and other sources accessed using sensors in the oral cavity and auxiliary devices.

### CROSS-REFERENCE TO RELATED APPLICATIONS

This application claims priority to U.S. Pat. App. No. 62/180,841 entitled “Oral Biosensor Alerts and Communication System” which was filed on Jun. 17, 2015 and which is incorporated herein by reference.

### STATEMENT REGARDING FEDERALLY SPONSORED RESEARCH OR DEVELOPMENT

Not applicable.

The present disclosure relates generally to smart, oral sensor devices and the integration of such with mobile communications, alerting and related technologies for both animals and humans, referred to herein as an ORAL SENSOR ALERTS AND COMMUNICATION (OSAC) SYSTEM.

### BACKGROUND

Oral saliva is increasingly recognized as an attractive diagnostic fluid. Saliva sampling involves simple and noninvasive collection methods which allow easy and fast diagnostic testing. Oral cavities contain salivary secretions, an abundant blood supply, lymph nodes, ingested pathogens, ingested toxins, ingested allergens, ingested drugs, ingested nutrients, and/or ingested food constituents.

The presence of various disease-signaling salivary biomarkers permits accurate reflection of normal and disease states in animals and humans. Information derived from the oral cavity is capable of augmenting, or possibly replacing blood sampling, and/or oral cavity information may be used as an efficient precursor before other more invasive medical diagnostics are employed. However, currently available methods for the detection of various salivary biomarkers are inefficient and do not alert or communicate information derived from biomarkers contained in the saliva in a rapid manner. Further, current usage of salivary biomarker information is immobile and uses patient care clinical diagnoses administered in large part through rudimentary swabs and similar means. Thus, there exists a critical need for improved devices, methods and systems in animal and human oral sensor and biosensor technology and its use for physiological and health information gathering, assessment, monitoring, and ultimately, health care and therapeutic assistance.

In addition, currently there is a profound lack of integration between a multitude of cross-linked technologies and skills when determining information regarding metadata diagnosis; with geometric tracking, multimedia, communication networks, analytics, alerting, and kinematics for individuals, team sports, organizational groups, animals and humans, which enhance health and performance. In addition, these current limitations restrict a multi-dimensional approach which could seamlessly measure individuals and animals with greater accuracy, convenience, yet far less intrusively. In addition the lack of integration between disciplines fails to address the growing need for the next level of metadata and biological tools which could provide early detection of diseases and medical disorders with greater accuracy as stated herein in the present invention.

### SUMMARY OF THE INVENTION

The present invention provides smart oral cavity devices, systems and methods relating thereto, as well as auxiliary devices and methods, for greatly improving animal and human well-being through innovations in such technology. The invention combines its enhanced, “smart”, oral sensor devices and methods with communications, software management, data management, instant and long term animal and human analyses, multimedia inputs, visualizations, geometric motion, tracking, kinematics, alerting, therapeutic, electronic medical records and other beneficial systems not previously available.

The OSAC system of the invention provides for communication systems and alerting technology that link a multitude of saliva-based biological information inputs together. This method of gathering biological information from oral devices provides the basis for a real-time or near-time snapshot of an animal or human's health and well-being.

Accordingly, an oral sensor alerts and communication system, methods and devices related to and used in conjunction therewith are provided which address the needs and provide the advantages outlined herein.

Also provided is an OSAC device according to the invention where oral sensor devices are connected to “smart” medical devices in response to alerts and/or signals from the OSAC system.

In an aspect of the invention, a device is provided which includes a smart sensor receptacle for a sensor. The receptacle is configured to be inserted in an oral cavity of an animal or human. The receptacle is configured to serve one or more functions within the animal or human's oral cavity wherein the one or more functions could be customized by physicians, veterinarians, patients, animal owners, users, and caretakers. The customizable functions can utilize mechatronics and can be integrated with sensors selected to measure and diagnose one or more medical health biometrics. OSAC imbedded oral cavity sensors could be utilized in conjunction with including, but not limited to, retainers to malposition teeth or jaws, corrective functions, providing a cosmetic or cleansing function, assisting in proper breathing, eating or swallowing, providing a tongue thrust dental guard function, pacifying infants, sports and recreational performance, animal sports and recreational performance, and other medical diagnostics, and analytics function. The device includes one or more sensors contained within or upon the receptacle or multiple receptacles.

In another embodiment of the invention, the OSAC system can streamline and integrate performance measurements such as, but not limited to, various geometric models, visualization, complex spatial-temporal relations, human and animal facial and physical relationships (individually and group), data associations (i.e., pixels, auditory, motion, optimum breathing, oral air-flow, accelerometers, gyroscope, metabolic biosensors, high-definition video capture, body-wearable sensors, RFIDs, readers, positioning, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple, various degrees of dynamic alerting, tracking, positioning, multi-media, analytics, historical and other comparative data inputs, communications and platforms). Collectively, these inputs can be synced and integrated with all forms of data capture. The OSAC system can provide important real-time or near time analytics in order to correct or modify motion, behavior for individuals, team sports or organizational groups for animals and humans.

In a further embodiment, the invention provides an OSAC system including the above-described smart receptacle, one or more sensors contained within, attached, or upon the receptacle and at least one interface with a network configured to utilize the information obtained from the one or more sensors.

It is understood by anyone familiar with the art that independent to wireless storage, the data could be stored in any OSAC device though any digital storage device, connector, or mechanism.

The invention provides, in another embodiment, a system which includes a device configured to be inserted in an oral cavity of an animal or human. The device includes a smart sensor receptacle for one or more sensors wherein the receptacle is selected and could be customized for any human or animal condition. For example, the receptacle can be selected from the group consisting of a horse-bit, a thermometer, a receptacle configured so that it cannot be swallowed, a receptacle for babies or adults with biosensors on one side and an RFID on the other side which is on the outside of a mouth, a customized teeth retainer which could be attached to a sports guard to enhance functionality and purpose. OSAC can include any combination of biosensors and RFID tags, micro- and nano-electronics, micro- and nano-enabled energy harvesting, micro- and nano-energy storage, micro- and nano-devices, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple frequencies located in the mouth or integrated outside of a mouth. In addition, other consumer products could include a dental OSAC insert in a pet's mouth, an animal toy which could be chewed but not swallowed and fastened securely within an animal's oral cavity. The system also includes one or more sensors contained within or upon the receptacle, and at least one interface with a network capable of utilizing the information obtained from the one or more sensors.

In yet a further embodiment of the invention, a method is provided for obtaining sensor data from a human and/or an animal. The method includes the steps of inserting a smart sensor receptacle configured to serve one or more functions within a human or an animal's oral cavity. The smart receptacle contains or receives within or upon it one or more sensors capable of providing information relevant to the health or a physiological characteristic of the human or animal. The method further involves activating or monitoring the one or more sensors to obtain or analyze the information relevant to the health or a physiological characteristic of the human or animal and transmitting at least some portion of the health or physiological information or analysis to a network capable of utilizing the information obtained. The one or more functions the smart sensor receptacle is configured to serve, e.g., is selected from the group consisting of replacing missing teeth or parts of teeth, repairing broken teeth, providing an aligning, fixing-malpositioned teeth or jaws, or other corrective function, providing a cosmetic and cleansing function, assisting in proper breathing, eating or swallowing, providing a tongue thrust dental guard function, providing a pacifying, function, providing a sports and recreational, such as flexible, inflexible, temporary, or permanent sensors and electronic for analytical and other functions and applications in order to provide accurate physiological information.

The recognition component in these systems and methods of the invention, often called a receptor, can use, e.g., biomolecules from organisms or receptors modeled after biological systems to interact with an analyte of interest. This interaction can be measured by a biotransducer which outputs a measurable signal proportional to the presence of a target analyte in the sample.

Another example would be monitoring blood components such as glucose, blood cells, medication, blood chemicals, etc. The bleeding could occur due to gum disease, oral trauma and injury, testing, teeth and gum cleansing such as flossing, water pick, blushing, anything that cause or induces bleeding, pin-pricks, etc. An OSAC insert might also contain cameras and lighting to visualize and capture the bleeding and to direct the OSAC sensor device to collect the blood from the bleeding area.

In another aspect of the method of the invention, the receptacle used in the above method includes a smart sensor receptacle for one or more sensors for example, but not limited to, a horse-bit, a thermometer, a retainer combination sports guard, a pacifier, an attachment to a tooth, an insert in a gum, etc., a receptacle which is configured not to be swallowed, a receptacle for babies or adults with biometric inserts with biosensors, sensors, communication capabilities including, but not limited, to camera, audio, thermal IR, multi-media, speakers, an RFID, etc. on the inside or outside of a mouth and an animal toy which is configured not to be swallowed, securely and strategically placed within an animal's or human's oral cavity.

In yet an additional aspect, the invention includes a diagnostic or other system for an animal or human. The diagnostic or other system includes a smart, wearable or attachable device, or a device insertable to the body or other component, but external to the oral cavity. The system also includes a receptacle configured for placement within the oral cavity of the animal or human. The oral cavity receptacle is configured to serve one or more functions within the oral cavity. These include or can be selected from the group consisting of replacing missing teeth or parts of teeth, repairing broken teeth, providing an aligning, fixing-malpositioned teeth or jaws or other corrective function, providing a cosmetic or cleansing function, assisting in proper breathing, eating or swallowing, providing a tongue thrust dental guard function, providing a pacifying function, providing a physiological, natural or grinding protective function, and providing a sports and recreational function, such as tongue piercing, bendable or flexible sensors and electronic, tattoos, or other functions, e.g., blood sample from oral bleeding for diagnostic purposes for diabetes. The system also includes one or more sensors located within or upon the oral cavity receptacle. The smart, wearable, attachable or externally insertable device is configured to obtain information from, provide information to, or both, the one or more sensors located within or upon the oral cavity receptacle. And, the one or more sensors or the smart, external device, or both, are configured to transmit the information to a network.

Also provided is a customizable development tool kit or platform for multiple OSAC purposes and functions and for building a diagnostic or other system to provide information such as, but not limited to hardware, and software integration, analysis, or alerts, for an animal, animals, human or humans. The kit includes customizable components to meet the needs of a consumer or user of the diagnostic or other system. The components include at least one sensor insertable or temporarily or permanently imbedded within or upon one or more oral cavity receptacles, and at least one oral cavity receptacle configured for placement within the oral cavity of the animal, animals, human or humans, at least one smart auxiliary device which is wearable, attachable or insertable externally to the oral cavity. The tool kit also includes at least one network unit configured to receive information from the at least one smart, auxiliary device, the at least one sensor, or both, and analyze, transmit or both, the information received. The components for selecting the at least one auxiliary device, the at least one oral cavity sensor receptacle, the at least one oral cavity sensor, and the at least one network unit are made available to the consumer or user to construct or have constructed a diagnostic or other system configured to obtain information, analysis or alerts customized to meet the specific needs of the consumer or user.

### BRIEF DESCRIPTION OF THE DRAWINGS

The presently disclosed subject matter will be better understood from reading the following description of non-limiting embodiments, with reference to the attached drawings, wherein below:

FIG. 1A is schematic depictions of an embodiment of the OSAC secure communication system and network of the invention where inserted oral sensors connect with GPS and other communication networks and devices of the invention for animals and humans, in accordance with embodiments of the present invention.

FIG. 1B is a schematic depiction which exemplifies an OSAC smart collar equipped with wireless communication (Bluetooth, etc.) capability and retrieves information in the pet's oral cavity according to the invention. The smart collar, accessories, or wearables transmits the oral cavity information to one or more remote PC laptops, tablets, smart-phones, or wearable communication devices through the owner's WiFi in accordance with embodiments of the present invention.

FIG. 2 is a schematic depiction which exemplifies an OSAC system according to the invention which communicates with a plurality of sensors, such as biosensors located within the oral cavity, receptors, transducers and processors and micro- and nano-electronics in accordance with embodiments of the present invention.

FIG. 3 depicts an embodiment of an OSAC communications system and networks where oral sensor or biosensor devices are permanently or temporarily inserted in an animal or human's oral cavity.

FIG. 4 depicts an embodiment of an OSAC communication system and network where oral sensor, e.g., biosensor devices (permanent or transitory) are fitted for animals (warm-blooded and cold-blooded), in accordance with embodiments of the present invention.

FIG. 5 depicts embodiments which exemplify an OSAC communications system and network wherein oral sensors connect with GPS and other communication networks and wherein oral sensor devices are inserted in humans in locations within the oral cavity, in accordance with embodiments of the present invention.

FIG. 6 depicts embodiments which exemplify the locations of OSAC oral sensor devices transitorily inserted in humans in accordance with embodiments of the present invention.

FIG. 7 depicts embodiments which exemplify an OSAC communications system and network where oral sensors such as various biosensors connect with GPS and other communication networks wherein the oral sensor devices are long-lasting implanted units for humans, in accordance with embodiments of the present invention.

FIG. 8 (A) depicts embodiments which exemplify utilities of an OSAC system for one or more pets, in accordance with embodiments of the present invention.

FIG. 8 (B) depicts embodiments which exemplify utilities of an OSAC system for a human, in accordance with embodiments of the present invention.

FIG. 8 (C) depicts embodiments which exemplify utilities of an OSAC system for one or more equines, mules or any other riding animal, in accordance with embodiments of the present invention.

FIG. 8 (D) depicts embodiments which exemplify utilities of an OSAC system for one or more sports, athletics or organizational participants, or one or more sports teams or organizational groups, in accordance with embodiments of the present invention.

FIG. 9 depicts a block diagram exemplifying an OSAC system which integrates various performance measurements in accordance with embodiments of the present invention.

FIG. 10 depicts embodiments which exemplify OSAC dynamic alerting software and secure networks, in accordance with embodiments of the present invention.

FIG. 11 depicts embodiments which exemplify an OSAC smart pacifier and its various communications and functions, used in accordance with embodiments of the present invention.

FIG. 12 depicts embodiments which dispense liquid medicine to infants, in accordance with embodiments of the present invention.

### DETAILED DESCRIPTION OF THE INVENTION

The foregoing summary, as well as the following detailed description of certain embodiments will be better understood when read in conjunction with the appended drawings. As used herein, an element or step recited in the singular and proceeded with the word “a” or “an” should be understood as not excluding the plural of said elements or steps, unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

As used herein, the term “smart” means a device or object that performs one or more functions of a computer or information system, such as data storage, calculation, Internet access and information transmission.

As used herein the terms “insertable”, “implantable”, “imbeddable”, “embeddable”, “temporarily insertable” “permanently insertable”, “temporarily implantable”, “permanently implantable”, “temporarily imbeddable”, “permanently imbeddable”, “temporarily embeddable” and “permanently embeddable” refer to means of securely inserting and attaching in or to, or fastening a device, such as being adhered to, cemented, affixed or otherwise securely attached to a surface or object.

As used herein, the term “receptacle” refers to a device or container that receives, retains, has within, or holds something.

Described in its broader respects, the Oral Sensor Alerts and Communication (OSAC) system of the invention includes a device configured to be inserted and securely attached in an oral cavity of an animal or human. The device includes a smart sensor receptacle for a sensor. The receptacle is preferably configured to serve one or more functions within the animal or human's oral cavity without being swallowed. The device also includes one or more sensors contained within or upon the receptacle, and also at least one interface with a network configured to utilize the information obtained from the one or more sensors or from one or more platforms providing additional information or capabilities networked with the system.

The system as described above may provide, e.g., one or more functions of the device including or selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological, natural or grinding protective function, and providing a recreational or sports function, health analytics, diagnostic analytics, performance analytics; integration of body sensors, health-devices, nano-particles, and sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision or analytics.

In certain embodiments, the system is set up wherein at least one of the one or more functions of the device is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological, natural or grinding protective function, and providing a recreational or sports function.

Additionally, the system may further include one or more sensors contained within or upon one or more receptacles located within or upon the animal or human's body networked with the oral cavity device.

In one particular embodiment, the system sensor receptacle is a pacifier and the one or more sensors include one or more sensors for temperature, blood pressure, core body heart rate, levels of a predetermined biologic, chemical or medication or their metabolites, optimum breathing, oral air-flow, accelerometer readings, gyroscopes, inertia-sensors, tracking sensors, sensors with input from cameras, videos, microphones, and speakers within the pacifier's plastic nipple encasement, or other parts of the pacifier or a pacifier component, face shield, or neck.

In additional embodiments, the pacifier may be configured so that sensor measurements are displayed on the pacifier. Further, in certain embodiments, the system also comprises one or more auxiliary sensor inputs from one or more of a camera, video, microphone, speakers, accelerometers, gyroscopes, or tracking sensors. For example, the above system may include one or more auxiliary sensor inputs configured to securely transmit sensor data through Bluetooth, GPS or other wireless means to one or more networked smart devices. The networked smart devices can include a controlling device to be monitored by and/or to alert parents or others of a change in diagnostics. This set up can permit the controlling device to be configured to notify one or more additional networked devices to send out bio-stats and alerts.

When the system includes a camera, it may preferably be configured to face away from the baby and to be activated remotely. Moreover, the camera may be set up to record and send out alerts when a predetermined stimulus, such as an unwanted presence, or change in readings, occurs.

Another component useful in this system is a network unit which includes a lockable device configured to be unlocked when authorized by a controller. For example, the lockable device can include a locking mechanism configured to disallow an unauthorized third party access to read sensor information or, when conditions are met, to allow such access. This system may also be set up to transmit information which optionally is shared with other networked devices.

In yet another aspect, one or more sensors are designed to be insertable within or upon the pacifier and constructed to be interchangeable or securely affixed to the pacifier. The system in which the sensor is insertable and is configured to be securely affixed to the pacifier may also be configured to be exposed to content within the oral cavity.

Alternatively, the system can be established so that the pacifier is protected by a thin layer of material or by an air pocket and outer layer using means well known to those skilled in this art.

In certain circumstances, a wireless transceiver chip is built into the pacifier device enabling the device to send and receive data, commands and other information.

And with yet another optional feature of this system, it is networked to dispense medications when instructed to do so based on alerts, signals or the like. To enhance the accuracy of the dispensing mechanism, in a preferred embodiment, the pacifier is calibrated for enhanced precision of measurement of the dispensed medications.

In another enhancement, the pacifier can be configured to be opened and dismantled for cleaning or storage or for other useful purposes. And in yet an additional enhancement, the system further comprises a secure clamp or other mechanical, electronic or magnetic lock, and/or the system can be configured to be remotely controlled.

The system can further include a pre-calibrated liquid cartridge configured to contain one or more medications which may be insertable into the pacifier device.

Alternatively, or additionally, the system may be established wherein pacifier accessories are configured to be attached to a feeding bottle to dispense and monitor medications, and to monitor feeding patterns, feeding times, durations, amounts, or liquid temperatures.

Thus, the pacifier may be configured to work in conjunction with an auxiliary device which is not inserted into the oral cavity. This auxiliary device may be configured to sense when an infant has taken a full or partial dosage, and dispense medication as directed by a controller, such as a networked program, a parent monitoring the system, or other. Examples of the auxiliary device include one or more of pacifier accessories, pacifier-straps, strap-buckles, clips, pacifier-holders, clothing, or a separate attachment for a camera, motion detector, tracking sensors, video, speakers, microphones, digital storage devices, or WiFi. The auxiliary device can be configured to sense when the infant has taken a full or partial dosage, and to dispense medication. The separate attachment can be configured to be fully or partially attachable to and removable from clothing, a crib, a toy, a blanket, a baby's person, a feeding-baby bottle, or a caretaker, for example.

As to particular examples of receptacles useable for the system, these include, but are not limited to one or more of a horse-bit, a smart thermometer, a smart gauge, smart dipstick, smart rod, smart stick, smart device to collect blood or saliva, a receptacle configured to be inserted but not swallowed, a receptacle for babies or adults with biosensors and an RFID, micro- and nano-sensors, micro- and nano-electronics, micro- and nano-enabled energy harvesting, micro- and nano-energy storage, micro- and nano-devices micro- and nano-programmable processors, micro- and nano-memory devices, micro- and nano-integrated power management devices, micro- and nano-programmable hardware, micro- and nano-wireless devices with communication capabilities across multiple frequencies located in an oral cavity or integrated outside of the oral cavity, and a device configured to be inserted into an animal or human's oral cavity, but not swallowed, one or more sensors contained within or upon the receptacle, and at least one interface with a network capable of utilizing the information obtained from the one or more sensors, one or more platforms, or one or more auxiliary devices or body integrations.

Specific examples of the system include: the smart sensor receptacle is a pacifier and the smart sensor receptacle is configured with WiFi connectivity, the system further includes one or more sensors for temperature or oxygen levels, the system further comprises tracking and camera functionalities, and the system further provides an alerting signal when outside a pre-set range; the smart sensor receptacle is a pacifier with full connectivity, the system further includes full server access and is configured for an analytical processing capability; the smart sensor receptacle is a full or partial retainer, the system further includes a smart mouth guard accessory, the one or more sensors includes sensors for temperature or oxygen levels, the system is further configured with WiFi connectivity and is configured to provide an alerting signal when outside a pre-set range; the smart sensor receptacle is a full or partial retainer, the system further includes a smart mouth guard accessory, the system is provided with full connectivity, full server access and is configured for an analytical processing capability comprising racing performance analysis.

The network units of the system include ones capable of utilizing the information obtained from the one or more sensors and having functions including, but not limited to, data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, analyzing historical or previous data from same or different individual or team, visualizing or presenting development platforms.

For example, the network can include units of a system for tracking an animal, animals, human or humans' biomedical condition integrated with an analytical or predictive capacity to determine or estimate possible points of origin, routes of travel, current location, or proximity to specific events or locations.

Additionally, the system can include network system units configured to track lost animals, humans, babies or adults as discussed elsewhere in greater detail herein.

While there is a focus on the sensors insertable and securely attachable in the oral cavity, the system can, and in many embodiments, does include at least one auxiliary sensor receptacle selected from, but not necessarily limited to, the group consisting of a horse-bit, a mouth-bit, a bit-guard, a lip-strap, a smart thermometer, a smart gauge, smart dipstick, smart rod, smart stick, smart device to collect blood or saliva, a receptacle configured to be sucked but not swallowed, a receptacle for babies or adults with biosensors, sensors, cameras, audio speakers, an RFID inside or outside of a mouth, micro- and nano-electronics, micro- and nano-enabled energy harvesting, micro- and nano-energy storage, micro- and nano-device, micro- and nano-timer, micro- and nano-programmable processors, micro- and nano-memory, micro- and nano-integrated power management, micro- and nano-programmable hardware, micro- and nano-wireless communication capabilities across multiple frequencies, sensors located in a mouth or integrated outside of a mouth, and an animal toy which is configured to be sucked but not swallowed.

Sensors may include sensors for a biologic, a biologically relevant molecule, temperature, blood pressure, pulse rate, blood oxygen level, respiration rate, gyroscopic measurement, accelerometer measurement, or kinematics, and the network capable of utilizing sensor information is configured to have the information transduced, amplified, or processed and a signal from the network transmitted through a RFID tag to an RFID reader on an accessory attached to or associated with a horse, the information comprising heart rate, oxygen level, gyroscopic measurement, accelerometer measurement and inputs for inertia-sensor, tracking sensors, camera, video, microphone, or speakers.

In a featured embodiment, the system can include one or more sensors placed within or upon horse-related equipment. Examples of such equipment useful for sensor placement in this embodiment include a headstall, headgear, an ear-pom, a blinker hood, a hackamore, a noseband, a cheese-band, a bridle, blinders, winkers, ornaments, phalerae, and sallongs.

The information utilized by the network can include one or more values integrating sensor data received by, e.g., the oral bit guard sensor from a central cardiovascular system.

The network units included in this aspect, include, but are limited to, one or more of measurements of performance, measurements of health, information obtained from biosensors, kinematics information, information obtained from cameras, information from sensors placed on blinker hood, nose-pieces, information from sensors attached to nose or other facial parts, information obtained from a heart-monitoring device, heart-rate, or respiration monitoring devices attached to horse equipment associated with the horse, information monitored by placing sensors on manure catchers, information from sensors attached to or associated with diapers attached to or associated with a tail or a tailbone of the horse, information from sensors attached to or associated with a leg, torso, neck, head or other part of the horse, information from instruments used to measure performance, information from accelerometers, gyroscopic measurements or inertia-sensors, information received from an RFID tag reader, information from sensors attached to or associated with a smart horse-rein, information received from sensors attached to or associated with inanimate objects around the horse or information from other horses.

The system includes one or more network units which can be configured to carry out a functionality including or consisting of signaling bi-directional transmissions to a secure server through one or more of WiFi, Bluetooth, GPS, and NFC, temporarily storing information in the smart device, and bi-directionally transmitting alerts to pre-selected devices or pre-selected personnel. Further network units employable in the system include or consist of, one or more RFID components, one or more cloud applications, a real-time or near-time slumber to alert mode, a manual control diagnosis mode, a programmed automated diagnosis mode, a geographic analysis mode, a species classification analysis mode, a disease specific or situational alerting mode, a function by which the one or more oral sensors is activated and inactivated by another sensor, device or remote controller, and transmission through WiFi or other wireless mode.

Also provided is a system comprising a device configured to be inserted in an oral cavity of an animal or human including a smart sensor receptacle for one or more sensors. The receptacle can be, but is not necessarily limited to, a horse-bit, a smart thermometer, a smart gauge, smart dipstick, smart rod, smart stick, smart device to collect blood or saliva, and other bio-sensors, a receptacle configured to be inserted but not swallowed, a receptacle for infants or adults with biosensors on one side and an RFID on the other side which is on the outside of a mouth, micro- and nano-sensors located in a mouth or outside of a mouth, and a receptacle configured to be insert but not swallowed within an animal's oral cavity. The system includes one or more sensors contained within or upon the receptacle. And the system includes at least one interface with a network capable of utilizing the information obtained from the one or more sensors, or from one or more platforms or one or more body integrations. The network capable of utilizing the information obtained from the one or more sensors includes one or more units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, comparative, historical or previous from same or different individual or team, data producing, visualizing or presentation development platforms.

The above described systems include those where the network includes a system of tracking an animal, animals, human or humans' biomedical condition integrated with an analytical or predictive capacity to determine or estimate possible points of origin, routes of travel or proximity to specific events or locations.

The above-described system includes, in a specific application, those wherein the receptacle includes a horse-bit, the horse-bit is configured with WiFi connectivity, the one or more sensors includes temperature, oxygen levels or respiration rate, and the system is further configured to provide an alerting function when outside a pre-set range.

The above-described system also include, in specific embodiments wherein the receptacle includes a horse-bit with full connectivity, the system also includes full server access and is configured for an analytical processing capability including racing performance analysis.

In additional embodiments, the system includes at least one auxiliary smart sensor receptacle not configured to be inserted in an oral cavity of an animal or human. The system can include a network configured to analyze one or more performance parameters of a team sport or group activity.

The network can be configured to analyze one or more performance parameters of horse racing, and the system can include a network configured to provide an electronic medical records functionality.

The system including the auxiliary smart sensor receptacle can also include a network including one or more RFID components, micro- and nano-electronics, micro- and nano-enabled energy harvesting, micro- and nano-energy storage, micro- and nano-devices, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple frequencies located in a mouth or integrated outside of a mouth, one or more cloud applications, a real-time or near-time slumber to alert mode, a manual control diagnosis mode a programmed automated diagnosis mode, a geographic analysis mode, a species classification analysis mode, a disease specific or situational alerting mode.

The system can include one or more medical devices or medication dispensers. Further enhancements include a fully integrated treatment facility, a system in which one or more sensors is activated by another sensor, device or remote controller, network modes including transmission through WiFi or other wireless modes, and systems in which at least one auxiliary smart sensor receptacle for a sensor is configured to serve one or more secondary functions within the animal or human's oral cavity.

In a further aspect of the invention, applicants have provided a device configured to be inserted and securely attached in an oral cavity of an animal or human. The device includes a smart sensor receptacle for a sensor, and the receptacle is configured to serve one or more functions within the animal or human's oral cavity without being swallowed. The device also includes one or more sensors contained within or upon the receptacle.

The one or more functions served by the receptacle of the device can include or consist of functions selected from the group consisting of, but not limited to, replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological, natural or grinding protective function, and providing a recreational or sports function, health analytics, diagnostic analytics, performance analytics; integration of body sensors, health-devices, nano-particles, and sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision and analytics.

In certain embodiments, the above device serves at least to provide one of the following functions: replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological, natural or grinding protective function, and providing a recreational or sports function.

Examples of particular devices embodying the invention include wherein at least one of the functions includes replacing missing teeth or parts of teeth and the receptacle is dentures, crowns, bridges, dental implants, permanent or temporary caps, fillings, fixed prostheses, prostheses, artificial teeth, prosthodontics, sealants, dental composites or bonds; wherein at least one of the functions includes repairing broken teeth and the receptacle is inlays, onlays and crowns, dental implants, permanent and temporary caps, fillings, fixed prostheses, prostheses, artificial teeth, prosthodontics, sealants, dental composites or bonds; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function and the receptacle is a pacifier; wherein at least one of the functions includes providing a physiological, natural or grinding protective function and the receptacle is a night guard or partial guard; wherein at least one of the functions is providing a recreational or sports function and the receptacle is a tongue piercing, bendable or flexible sensory unit, electronic insert, stretch stripes, adhesive stripes, transdermal patches or tattoos.

In describing the nature of certain of the device receptacles which often have a conventional dental device aspect to their functionality, the receptacle can include permanent or temporary caps, implants, night guards, partial guards, crowns, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof.

The device receptacle is insertable, e.g., by micro- and nano-clips, frames, brackets, sealants, dental composites, bonds, adhesives, adhesive strips, cements, wires, bands, glues, embedment, injection, printing, tattooing, or any combination thereof.

In these devices, the receptacle can be configured to be removable, self-removable, self-installable, coverable with an air-tight material, and configured so that the one or more sensors is integral to the receptacle.

The sensors in the provided devices can include, e.g., sensors of blood pressure, core body temperature, heart rate, levels of a predetermined biologic, chemical or medication or their metabolites, sensors to measure a physical property, including one or more sensors which measure a physical property including or consisting of temperature, blood, pressure, teeth pressure, ionic conductivity, airflow, images, optical density, alterations to the oral cavity, surrounding muscle tone, muscle weakness, heart rate, heart rhythms, respiration rate, spectrophotometry, electromagnetic spectrum, gamma waves, X-ray waves, ultraviolet waves, visible waves, infrared waves, terahertz waves, microwaves, radio waves, electrical waves, sound waves, magnetic waves, ultrasonic waves, magnetic resonance, magnetic field, electro- or magnetic-encephalography, functional magnetic resonance imaging, optical topography, global positioning or tracking, accelerometer activity, gyroscopic activity, kinematic activity and radiation wave activity.

The sensors in the provided devices can include sensors which measure a salivary, blood, lymph node, bone, or tooth constituent, sensors which measure a predetermined biologic, sensors which measure a predetermined biologic including or consisting of DNA, RNA, telomeres, methylated or otherwise modified DNA or RNA, proteins, immunoglobins, antibodies, histones, peptides, modified proteins, neuro-peptides, pigments, and enzymes, sensors which measure dissolved gases, including oxygen, carbon dioxide, carbon monoxide, ammonia, sulphur, or an alcohol-containing gas, sensors which measure a lipids profile, sensors which measure a chemical molecule, sensors which measure a salt, an alcohol, a metabolite, an anion, a cation, water, a sugar, a protein, or a lectin, sensors which measure a drug or a medication, sensors which measure cells, the one or more sensors measures cells, cancerous cells, biomarkers for an oral or systemic infectious disease, biomarkers for drug abuse, biomarkers for a metabolic disease, biomarkers for malnutrition, biomarkers for obesity, biomarkers for a cardiovascular disease, biomarkers for atherosclerotic, biomarkers for infection, biomarkers for auto-immune and other immune diseases, biomarkers for stroke, biomarkers for AIDs, biomarkers for multiple sclerosis, biomarkers for periodontal diseases, biomarkers for brain-function disorders, dementia, memory loss, depression, mental disease, Alzheimer's disease, mentally-challenged disorders, nervous system disorders, tracking or wandering, and other psychology and neurological disorders, biomarkers for bleeding, head and neck injuries, biomarkers for Sjogen's syndrome, biomarkers for oxidative stress, biomarkers for allergies, biomarkers for cancer, biomarkers for skeletal and muscle diseases, biomarkers for genetic diseases, biomarkers for renal diseases, biomarkers for osteoporosis, biomarkers for fatigue, biomarkers for stress, biomarkers for sleep deprivation or sleep apnea, biomarkers for fertility, pregnancy, ovulation, and reproductive system disorders, biomarkers for cystic fibrosis, biomarkers for respiratory or pulmonary diseases, biomarkers for diabetes and ketoacidosis, biomarkers for inflammation, biomarkers for age-related diseases, biomarkers for dehydration, biomarkers for halitosis, biomarkers for alcohol consumption, alcoholism or drug consumption or drug addiction, biomarkers for hypoxia, smoking-related diseases, toxins, or pollutants, biomarkers for poor-gait, biomarkers for Crohn's disease, biomarkers for dental caries, biomarkers for blood and circulatory disorders, biomarkers for ear, nose, and throat diseases, biomarkers for taste, Ageusia, Hypogeusia, or Dysgeusia, biomarkers for bad-breath related diseases biomarkers for chewing or mastication, biomarkers for digestive disorders, biomarkers for hepatic diseases, spleen, gall-bladder and pancreatic diseases, biomarkers for urinary system disorders, biomarkers for integumentary system diseases, biomarkers for endocrine, lymphatic, and excretory diseases, sensors which measure a cell surface component or a cellular marker or component, sensors which measure a pathogen or a microbe, sensors which measure administered foreign materials, medications, diagnostic molecules, drugs, biologically sensitive, derived, bio-mimics, or bioengineered molecules, sensors which measure an ingested molecule or its metabolite, including wherein ingested molecule is a pathogen, a microbial, an ingested toxin, an ingested allergen, an ingested food constituent, including a nutrient, a micronutrient, a fat molecule, a carbohydrate molecule, a sugar molecule, a protein molecule, or an amino acid, sensors which measure ingested medications, ingested foreign material, ingested drugs, an ingested diagnostic molecule, an ingested biologically sensitive molecule, an ingested nanoparticle, an ingested derived molecule, a biomimic, or an ingested bioengineered molecule, sensors which interact with at least one disease-related biomarker, such as, e.g., a disease-related biomarker related to a disease diagnosable by a sensor which includes or consists of sensors of blood pressure, core body temperature, heart rate, levels of a predetermined biologic, chemical or medication or their metabolites.

The device as herein described can be securely attached within the oral cavity of an animal or human by a number of means, including one or more of being fixedly inserted, imbedded, fitted, fixed, implanted, fastened, joined, associated, coupled, linked, banded, united, mounted, combined, glued, adhered, cemented, or firmly connected by mouth, e.g., lips, teeth, etc., or hands or parts or accessories thereto of either.

In a particular embodiment, the device can include an interface with at least one sensor or nanoparticles not located within the oral cavity.

The systems and devices described as part of the OSAC system as laid out in this application can be applied to obtain extensive sensor data and analysis, providing much needed information and assistance, as detailed herein. In particular, a method for obtaining sensor data from an animal or human is provided. The method includes the steps of locating or inserting a device configured to be inserted and securely attached in an oral cavity of an animal or human. The device includes a smart sensor receptacle for a sensor, the receptacle being configured to serve one or more functions within the animal or human's oral cavity without being swallowed. In the method, the smart receptacle is inserted already containing, or, alternatively, receives one or more sensors after insertion, capable of providing or receiving information or analysis relevant to the animal or human. The method includes activating and/or monitoring the one or more sensors, and transmitting or receiving at least some portion of the information or analysis to, from or among a network or networks capable of utilizing the information or analysis.

In the above-described method, the one or more functions of the receptacle can include or consist of replacing missing teeth or parts of teeth, repairing broken teeth, providing an aligning, fixing-malpositioned teeth or jaws or other function, providing a cosmetic or cleansing function, assisting in proper breathing, eating or swallowing, providing a tongue thrust dental guard function, providing a pacifying function, providing a recreational or sports function, providing a physiological, natural or grinding protective function, or providing a function related to health analytics, diagnostic analytics, performance analytics; integration of body sensors, health-devices, nano-particles, sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, or providing functions with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement or greater precision and analytics.

In a particular aspect of the above method, at least one of the one or more functions of the receptacle includes or is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological, natural or grinding protective function, and providing a recreational or sports function.

In various embodiments of the method, information can be transmitted securely to a plurality of remote devices monitoring the animal or human, or information can be transmitted securely to a plurality of remote devices monitoring a plurality of animals or humans.

The network capable of utilizing the information obtained from the one or more sensors can include or consist of one or more network units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, comparative, historical or previous from same or different individual or team, visualizing or presentation development platforms.

The method can include network units which utilize preset ranges, dynamic preset ranges, or degrees of alerts from preset ranges for medical or performance analysis. Additionally, the described method can include network units which utilize biosensor or sensor measurements for pre-integration and post-integration analyses, as known by those skilled in such arts.

The method can also involve inserting the receptacle into the gums and measuring blood chemicals using oral or gum bleeding.

In a particular embodiment of the invention, a method is provided for monitoring a drug treatment, medication usage, food consumption, exercise, behavioral, or any other program or activity having a durational component. The durational component can be of very short duration, such as a single administration or meal, or it may involve an extended duration of minutes, hours, days, years, etc. In this embodiment, the method includes monitoring at least one smart sensor located in an oral cavity of a human or animal, or at least one smart auxiliary sensor not located in the oral cavity but in communication with the oral cavity sensor, before, during or after some aspect of the durational component is administered, consumed or undertaken, or some combination of such monitoring, thereby obtaining sensor data of one or more parameters of the durational component for transmission, analysis or both. For example, this method may be employed to monitor the efficacy or safety of a drug treatment or medication regimen, by monitoring before, during or after, or some combination thereof, administration of the drug or medication over a specified course of time. The sensor may track, e.g., the rise or fall in concentration of a biomarker which reflects the presence and/or severity of a disease for which the drug is being administered to counteract.

In another example, the method in this aspect includes monitoring at least one parameter of food consumption of a human or animal. Thus, here, at least one parameter of the food consumption is monitored before, during or after, or some combination thereof, the food is consumed over some course of time. An example of this aspect may include keeping tabs on ingested nutritional components being fed to a herd of animals to monitor whether they are receiving the right balance of nutrients from a particular meal, or over a feeding season.

In another embodiment, a diagnostic or other system is provided which includes a device configured to be inserted and securely attached in an oral cavity of an animal or human, wherein the device includes a smart sensor receptacle for a sensor. The receptacle is configured to serve one or more functions within the animal or human's oral cavity without being swallowed, and the smart receptacle is configured to contain or receive one or more sensors capable of providing or receiving information or analysis relevant to the animal or human. The system further comprises a smart auxiliary device which is wearable, attachable or insertable externally to the oral cavity. The auxiliary device is configured to obtain information from, provide information to, or both, the one or more sensors contained in, on, or received by the oral cavity receptacle. The one or more oral cavity sensors or the auxiliary device, or both, are configured to transmit or receive the information or analysis to or from a network or networks. This system can include an auxiliary device which is also configured to contain or receive one or more sensors.

The one or more functions can include or be selected from the group consisting of replacing missing teeth or parts of teeth, repairing broken teeth, providing an aligning, fixing-malpositioned teeth or jaws, or other corrective function, providing a cosmetic or cleansing function, assisting in proper breathing, eating or swallowing, providing a tongue thrust dental guard function, providing a pacifying function, providing a recreational or sports function, providing a physiological, natural or grinding protective function, or providing a function related to health analytics, diagnostic analytics, performance analytics; integration of body sensors, health-devices, nano-particles, and sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement or greater precision and analytics.

In certain embodiments, at least one of the one or more functions is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological, natural or grinding protective function, and providing a recreational or sports function.

In certain embodiments of this system, the animal is a pet, including a dog or cat, or other animals. In embodiments of this system involving pets, the auxiliary device can be a smart collar. Moreover, in systems involving pets, in specific embodiments, the system can include a database compilation of one or more attributes of animals having at least one similar characteristic to the pet and which connects via WiFi to an accessible portal and wherein the system provides diagnostic information regarding the pet. Similarly, for pets, the system can include a database compilation of one or more of the pet's biological or physiological attributes.

Generally, the system can include a historic database of the animal or human as to one or more characteristics from which comparisons or analyses are configured to be made, or a database of animals or humans having a common characteristic to the animal or human on which the smart device is located and for which a predetermined comparison is configured to be made.

In another embodiment, the auxiliary device comprises a smart device configured to be associated with one or more team members of a team wherein the auxiliary smart device comprises one or more sensors configured to obtain information from the one or more team members and is configured to transmit the information or analysis derived therefrom directly or indirectly to a network.

In one embodiment of this system, the auxiliary oral cavity device can be a mouth guard and the one or more auxiliary device sensors can measure, e.g., oxygen levels or heart rate, analyze fatigue, such as the fatigue of an individual team member, or the fatigue of a composite of a plurality of team members. The system can also be configured to analyze kinematics, such as where the network is configured to analyze the kinematics of an individual member of a team or group, or a composite of a plurality of team or group members.

In another OSAC system, the system includes a device configured to be inserted into an oral cavity of an animal or human. The device includes a smart sensor receptacle for one or more sensors, and the receptacle includes a retainer or retainer-like device, a smart mouth guard accessory which is configured to attach and detach to the retainer or retainer-like device, and the system is provided with data storage or full connectivity capabilities.

The system can be set up for use with an individual to obtain information from the individual and transmit it or analysis derived from it directly or indirectly to a network.

The system network can interface with a mobile device which in turn provides sensor information or analysis to the individual user, who then receives information feedback regarding a physiological characteristic of a current activity he is engaged in, such as running, jogging, walking, sleeping, or a physical characteristic involved with playing a sport.

The system may utilize a network configured to analyze one or more performance parameters of a team sport or group activity. It may also work with a network configured to analyze one or more performance parameters of horse racing. Additionally, in an additional embodiment, the system can include a network configured to provide an electronic medical records functionality.

The system as described above can include full server access and the system can be configured to analyze individual or team sports performance as it relates to various body components and sensors.

In one option of the system, the system includes one or more of a digital storage device or full connectivity capability configured to analyze individual and team sports performance as it relates to various body component sensors for post-play analysis and review.

As developed by applicants, in certain embodiments the invention is provided as a customizable tool kit or platform for building a diagnostic or other system to provide information, analysis or alerts for an animal, animals, human or humans. The kit or platform of customizable components serves to meet the needs of a developer, consumer or user of variations of the OSAC system. The components include at least one sensor insertable within or upon an oral cavity receptacle configured for placement in the oral cavity of the animal, animals, human or humans, at least one oral cavity receptacle configured to contain or receive the sensor, optionally, at least one smart auxiliary device which is wearable, attachable or insertable externally to the oral cavity, and at least one network unit configured to receive information, analysis or alerts from or transmit information, analysis or alerts to, the at least one oral cavity sensor or the at least one auxiliary device, or both, and analyze, transmit, or both, the information, analysis or alerts obtained or received. The components for selecting the auxiliary devices, the oral cavity sensor receptacles, the sensors, and the network units are made available to the developer, consumer or user to construct or have constructed an OSAC system configured to obtain or transmit information, analysis or alerts customized to meet their specific needs.

In an embodiment of the OSAC system, the tool kit or platform comes in a variable grouping of preselected sets of kit or platform components or modules of components for constructing the OSAC system using the kit or platform, and may come together with instructions for building the desired system. And yet further, in certain embodiments, at least one smart auxiliary component is present in the tool kit or platform.

The tool kit or platform as outlined above, e.g., can be designed for a sports function, health analytics, diagnostic analytics, performance analytics; integration of body sensors, health-devices, nano-particles, sports and performance sensors on inanimate objects and sports equipment; customizable developers' tool kit for biosensors, sensors, performance, medical analytics, oral and systemic diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision and analytics.

The tool kit or platform can be designed for an animal, including a pet, e.g., where the pet is a dog or cat, and also, where the smart auxiliary device is a smart collar.

The tool kit or platform in an embodiment includes one for a system including network units involving a historic database of the animal or human as to one or more characteristics from which comparisons or analyses are to be made, or a database of animals or humans having a common characteristic to the animal or human on which the auxiliary device is to be located and for which a predetermined comparison is to be made.

The tool kit or platform can be designed for a system for an animal such as a pet, in which the auxiliary device comprises a smart collar which connects via WiFi to an accessible portal and wherein the system provides information or analysis regarding the pet obtained from or transmitted to one or more sensors located within the oral cavity of the pet.

The tool kit or platform can be configured for a system which includes a database compilation of one or more of the pet's biological or physiological attributes, or wherein the system includes a database compilation of one or more attributes of animals having at least one similar characteristic to the pet.

The tool kit or platform can also be put together for a system wherein the auxiliary device is associated with one or more team or group members of a team or group, includes one or more sensors also associated with the one or more team or group members, and the auxiliary device is configured to transmit information from the one or more sensors to a network.

The tool kit or platform can be provided for a system in which the oral device is a mouth guard and the one or more sensors measures oxygen levels or heart rate, the network analyzes fatigue, such as the fatigue of an individual team member, the fatigue of a composite of a plurality of team members, the network analyzes an aspect of a team or group performance criteria of a team or a group, or the network analyzes one or more components of horse racing performance.

In another embodiment, the tool kit or platform provides the necessary components for building a system wherein the oral and/or the auxiliary device is configured to be associated with an individual and to transmit information to a network, e.g., in which the system has a network which interfaces with a mobile device. The tool kit or platform in this embodiment is designed to provide a system including a network which interfaces with a mobile device which provides sensor information to the individual user, such as to give feedback regarding a biological or physiological characteristic of a current activity. The current activity can be, e.g., running, jogging, walking, sleeping, and a physical characteristic of playing a given sport.

The tool kit or platform can also include the components and, optionally instructions, e.g., to provide a system with an electronic medical records functionality.

Applicants have also devised a method for monitoring blood components of an animal or human. The method includes the steps of inserting and monitoring a device or monitoring an already inserted device which is configured to be inserted into an oral cavity of an animal or human. For this method, the device includes a smart sensor receptacle for one or more sensors and the receptacle preferably is configured to function without being swallowed. The smart receptacle contains or receives one or more sensors capable of providing or receiving information or analysis related to the animal or human's blood components and it uses the sensors to monitor or analyze one or more aspects of the blood components. The information or analysis is then transmitted to, from or among a network or networks capable of utilizing the information or analysis. In certain embodiments, the device is also configured to collect blood from bleeding due to gum disease, oral trauma and injury, testing, teeth or gum cleansing, flossing, water picking, brushing, pin-pricking, or other inducements of bleeding. Additionally, the device used for monitoring blood components can be configured to be inserted into the oral cavity to be bathed in blood to measure blood glucose levels, blood composition, medication, blood chemistry, or other characteristics. Further, the device can be configured to collect the blood from the bleeding area to detect glucose, blood chemicals, medication, or other blood-related characteristics.

The method for monitoring blood components can include data storage, e.g., through a digital storage device, connector, or other medium integral to or separate from the blood monitoring device.

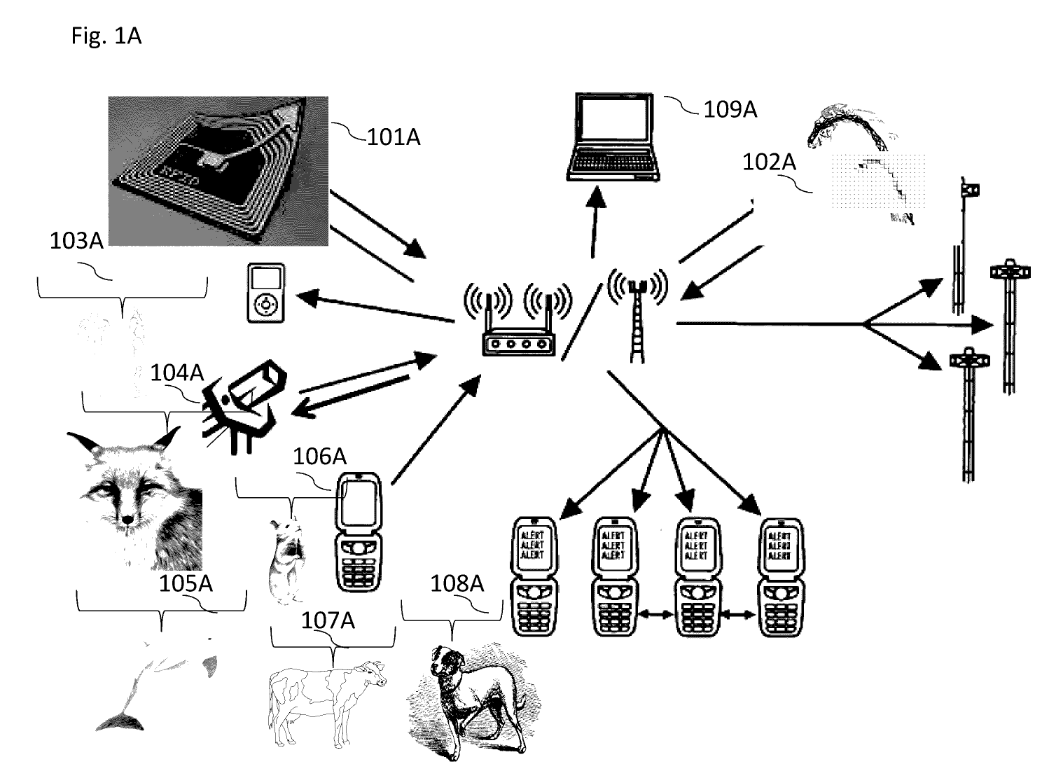

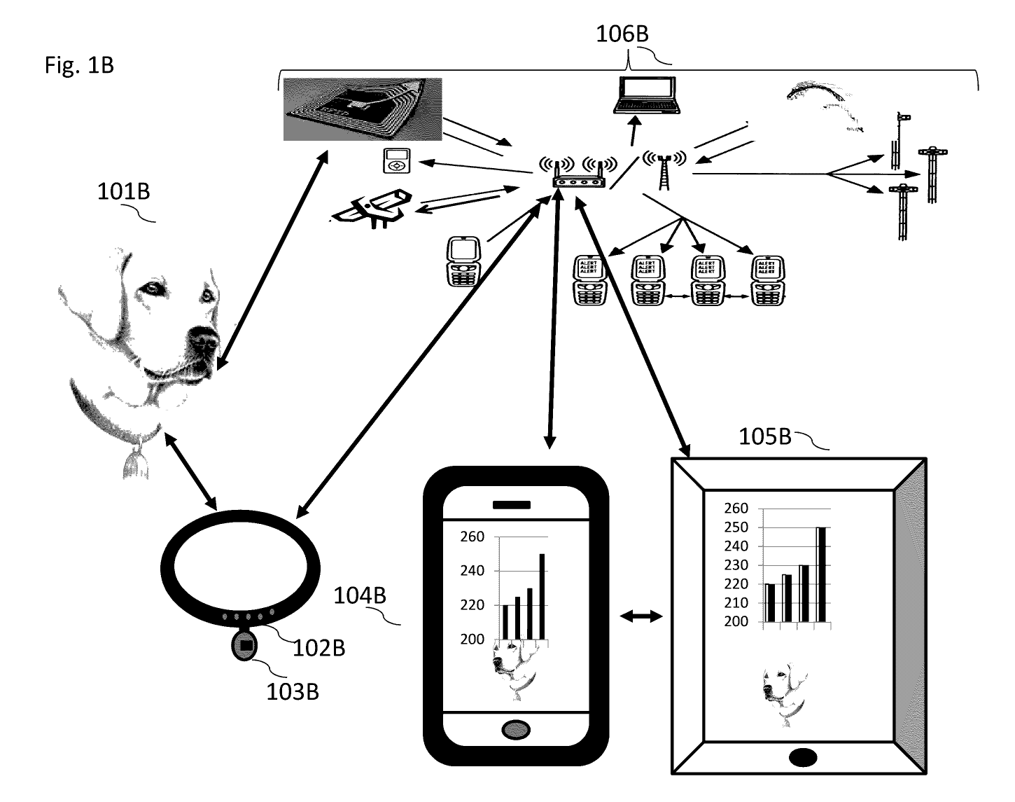

FIG. 1A. 101A includes addition, monitoring, and management software implemented in order to track a multitude of Radio Frequency Identification (RFID), near field communication, micro- and nano-communication devices, micro- and nano-electronics, etc. data inputs. Active and/or passive, and/or a combination of RFIDs use electromagnetic signals to uniquely distinguish and identify a mobile “TAG” device or stationary “TAG” device. The active RFID identification system tag has its own power source, enabling the unit to broadcast an identifying signal. This extends the range of the tags and capability of communicating advanced data, such as location and other pertinent information, and broadcasts an identifying signal. Passive RFID tags are not powered and rely on active signals from location transmitters for their response. RSSI (Received Signal Strength Indication) is an algorithm that determines the location of an active tag by measuring the power of the radio signals. TDOA (Time Difference of Arrival) is an algorithm that determines the location of active tags by measuring the power of radio signals in real-time. Some RSSI systems have choke-point capabilities that provide an instantaneous notice that a tag has passed a certain point. 102A is an example of an oral implant, which communicates with one or more wireless devices 103A, networks, and subsystems (WiFi, satellites, cellular, etc.) which interface and communicate with various animals or humans as exemplified by 104A-109A.

FIG. 1B, in addition to the aforementioned, in embodiments of the described OSAC system, also communicates through WiFi or other wireless methods. 101B represents a dog and/or other pet, a smart collar 102B and/or smart tag or attachment which can be added and/or attached to an existing collar such as in the form of an accessory attached to the collar 103B. 103B and 102B may also be equipped with WiFi or Bluetooth, GPS, and such. In this embodiment, these connected devices are remotely activated, inactivated, and accessed by the owner/master and others, such as a veterinarian, through his/her, PC, laptop, tablet, or any smart device with an Internet connection as shown by 104B-105B. In addition, as elsewhere described, a master is able to monitor and view his pet's metabolic diagnostic statistics through these devices as represented by 104B-105B. 106B depicts an embodiment of the OSAC system and network according to the invention, as well as the oral cavity sensor inserted into a pet. The collar may comprise more than one active or passive RFID device, wireless communications, receivers, microphones speakers, camera, and the like.

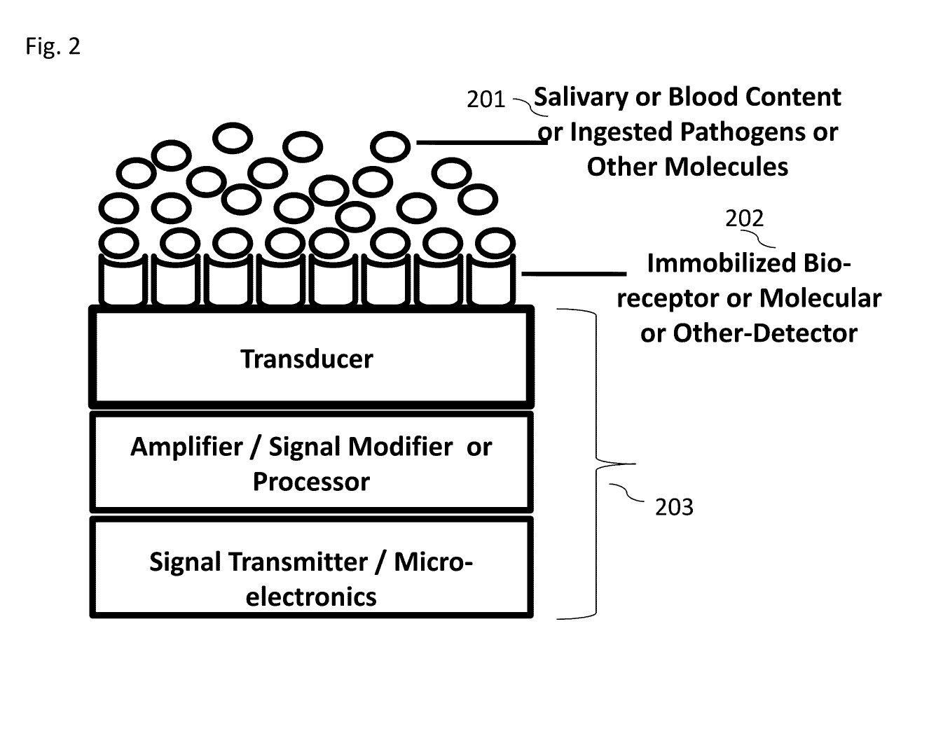

FIG. 2 exemplifies the structure of a sensor or biosensor 201 as may be used in embodiments of the invention. At the top, a biologically active or non-inert surface contains, e.g., biosensors—bio-detectors, bio-receptors, or biologically sensitive materials, and is exposed to biomarker molecules (see, e.g., Table II). Bio-detectors, bio-receptors, or biologically sensitive materials 203 include, but are not limited to, biologically-derived materials, bio-mimics, and chemical and bioengineered molecules. The sensors interact with biological molecules or biomarkers, and this recognition is converted by transducers 202 to a signal that is more readily measured and quantified. A transduced signal is transferred to a signal amplifier or modifier, which transfers the signal to a wireless transmitter in this embodiment. These biosensors could be custom-made by 3D printing.

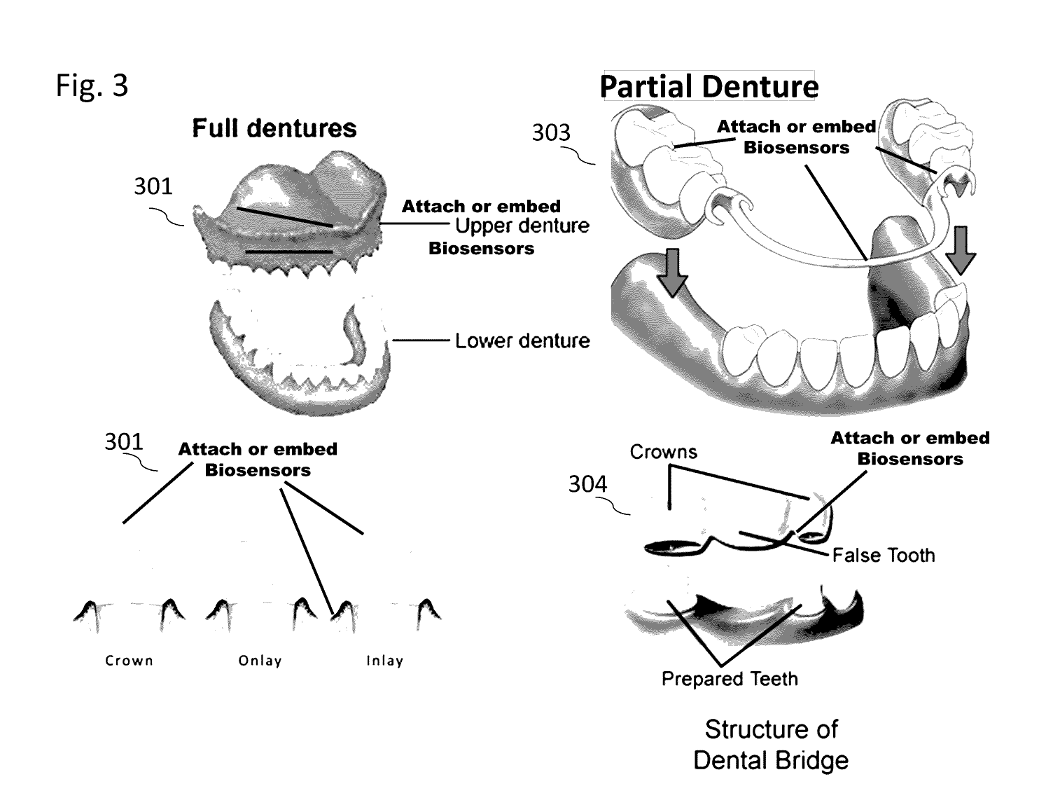

FIG. 3 depicts human oral cavity receptacles which include temporary, fixed, or permanent dental wearables 303, exemplified by 302. 301(a)-301(b) depict frontal and lateral views of the oral cavity; the lateral view also shows lymph nodes that form part of the oral cavity, as defined herein; and secretions that drain into these oral nodes. Certain items described as “wearables”, or other sensor devices may also be used in conjunction with the smart, oral cavity sensor receptacle devices of the invention. The anterior limit is superior, and the inferior lips extend to the larynx as the posterior limit. The upper limit is the palate or upper jaw, and the lower limit is the muscle surrounding the lower jaw. The submandibular lymph node gets secretions from the cheeks, lower lip, gum, and anterior tongue. The submental lymph nodes get secretions from the lower lip, mouth floor, and tongue apex. The tonsillar lymph nodes get secretions from cheeks, gums, and tongue. All these lymph nodes form parts of the oral cavity.

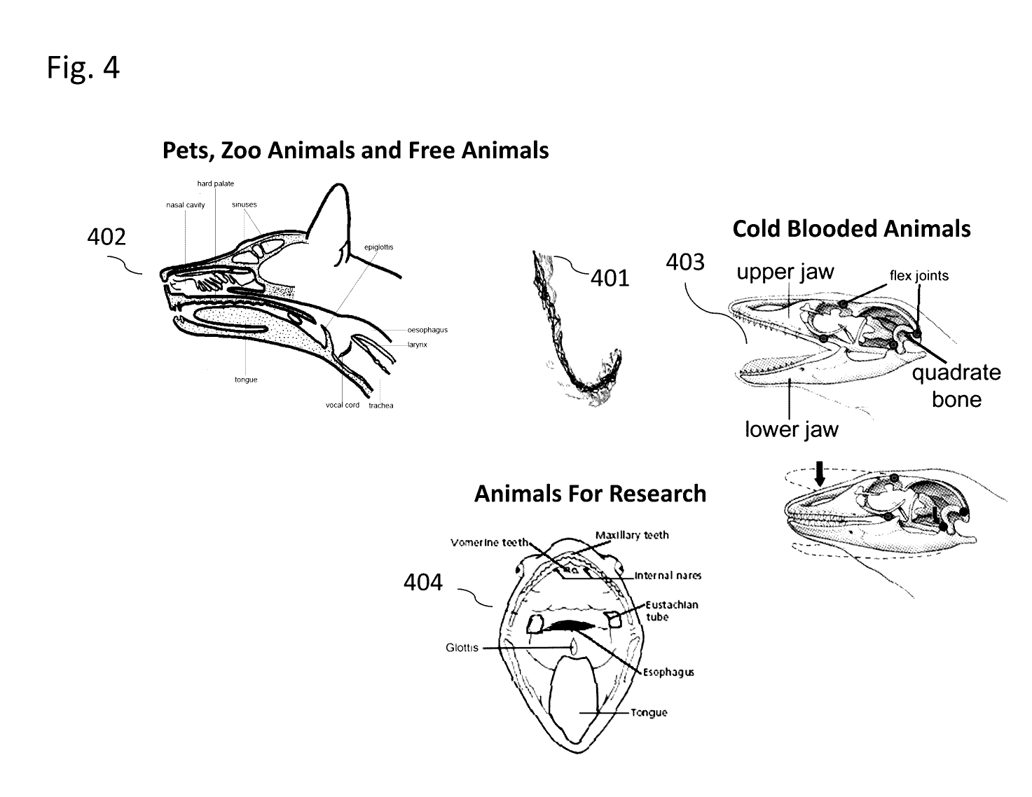

FIG. 4. Animal Oral Cavity: Oral wearables 401, and the mammalian oral cavity. The lateral views representative of the chimpanzee, human, horse, cow, dog, and cat are shown 402. The interior view of the pig is also shown. All of these animals have oral cavities that are substantially the same as the human oral cavity. The anterior limit is defined by the lips, and the oral cavity extends up to the larynx at the posterior end. The jaws 404, like for humans, define the oral cavity in these animals. This oral cavity has the upper jaw and lower jaw forming its upper and lower limits, also similar to humans. Any animal that has mouth parts has an oral cavity as used herein. Similar to humans, in various embodiments, biosensor dental devices are implanted, i.e., adhered to, cemented, affixed or otherwise attached to parts of the oral cavity containing them. These biosensors may be temporarily or permanently inserted via an appropriate oral sensor receptacle. Also depicted are views of the vertebrate animal oral cavity 403 including lateral views of their oral cavity. Representative views of the oral cavities of the bird, frog, snake, fish, and mammals are also shown here for research 404.

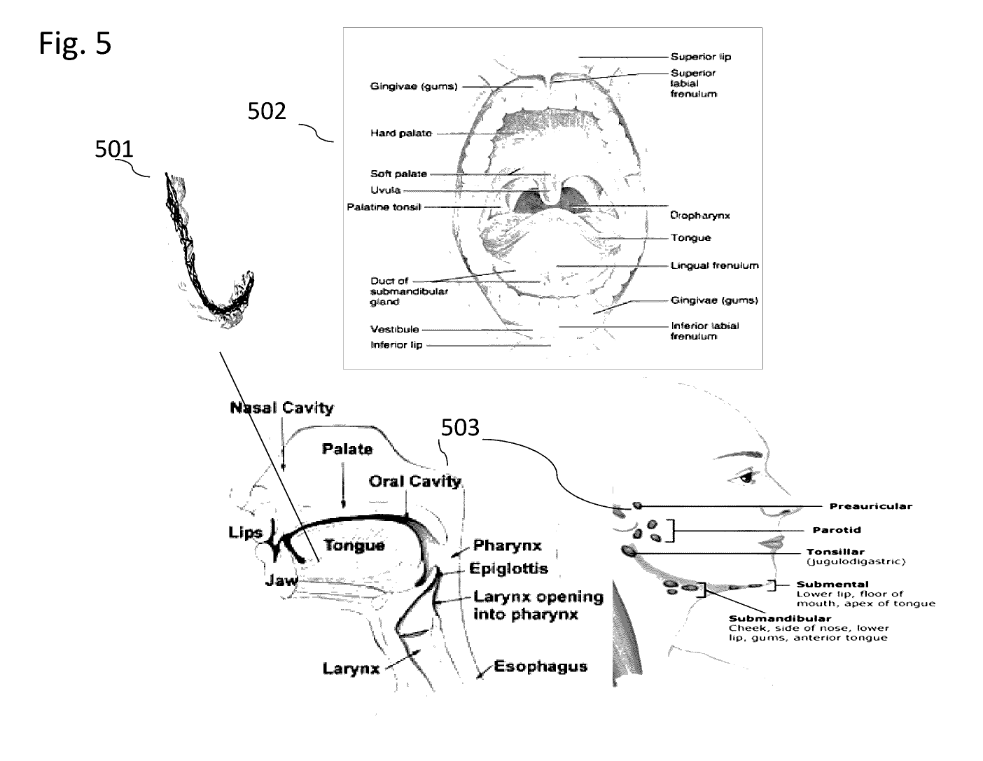

FIG. 5 shows examples of various places within the oral devices where biosensors can be attached in accordance with embodiments of the invention. Biosensors can be attached to dental devices through micro- and nano-clips, frames, brackets, adhesive, cement, embedment, or by some other method in preferred embodiments. In 501, these biosensor attachments through micro- and nano-clips, frames, or brackets can be removed to either clean or to be exchanged for another biosensor. In certain embodiments, biosensors are adhered, embedded, or cemented to the dental device, palate, inside of cheeks, lips, tongue, sublingual cavity, gums, or teeth, as represented by the human illustrations 502 and 503. It is understood by anyone familiar with the art that fixed, “cemented” biosensor devices can be permanent or can be placed in the oral cavity for a few seconds or minutes to several months, etc. In other embodiments, biosensor devices are implanted in the palate, inner cheeks, tongue, gum, sublingual cavity, or even in the jaw bone or muscles. Biosensor devices may be permanent and intended to be placed in the oral cavity for several years. Lymph nodes offer a better site for location of some biosensors, such as, e.g., detectors of ingested pathogens, cancerous cells, and other immune response molecules, according to certain embodiments of the invention.

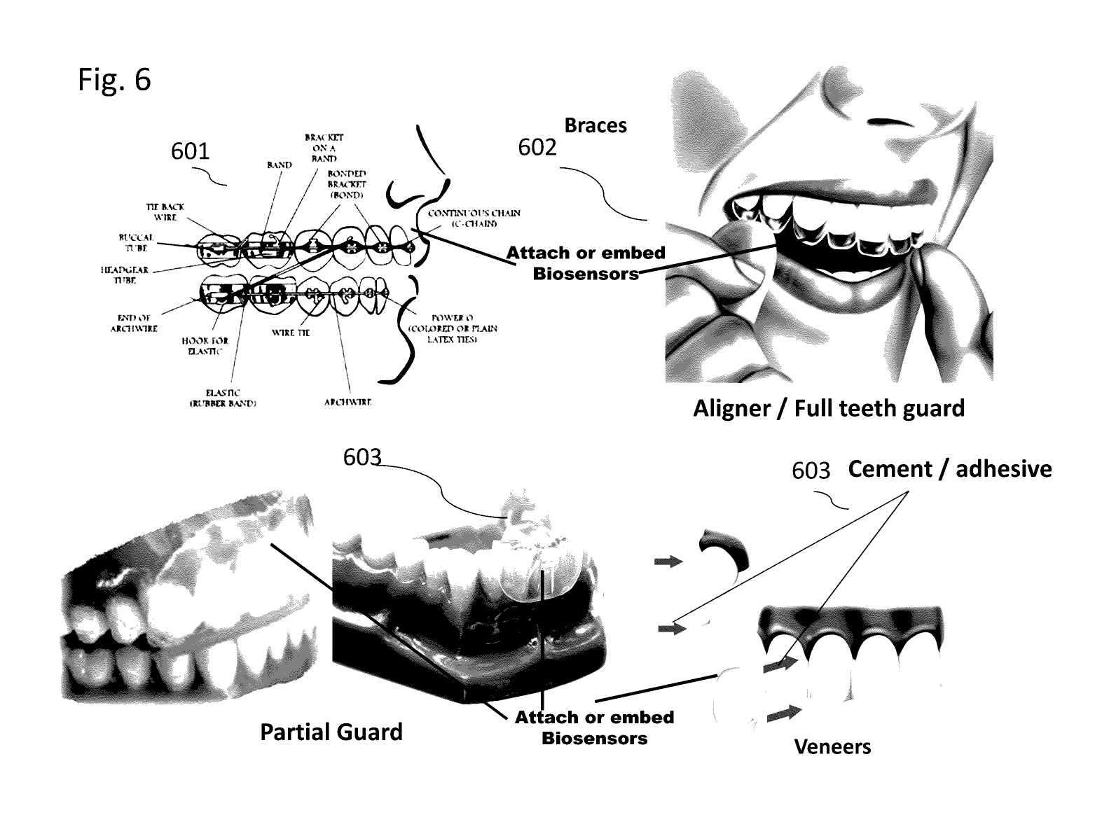

FIG. 6. Unfixed dental devices 601 are defined as ones not permanently attached to the jaw bone, but as possibly attached to the gum or teeth. Temporary biosensor mouth guards 603 have a generally shortened life span compared to fixed devices, but they may be placed in the oral cavity for from several minutes to several months (but typically are not designed for placement, e.g., for several years). Biosensors are optionally attached to or embedded in these devices. Some examples of unfixed dental devices are partial and full dentures, bridges, crowns, onlays, inlays, full or partial veneers, full or partial aligners, and guards 602. These biosensors could be custom-made by 3D printing.

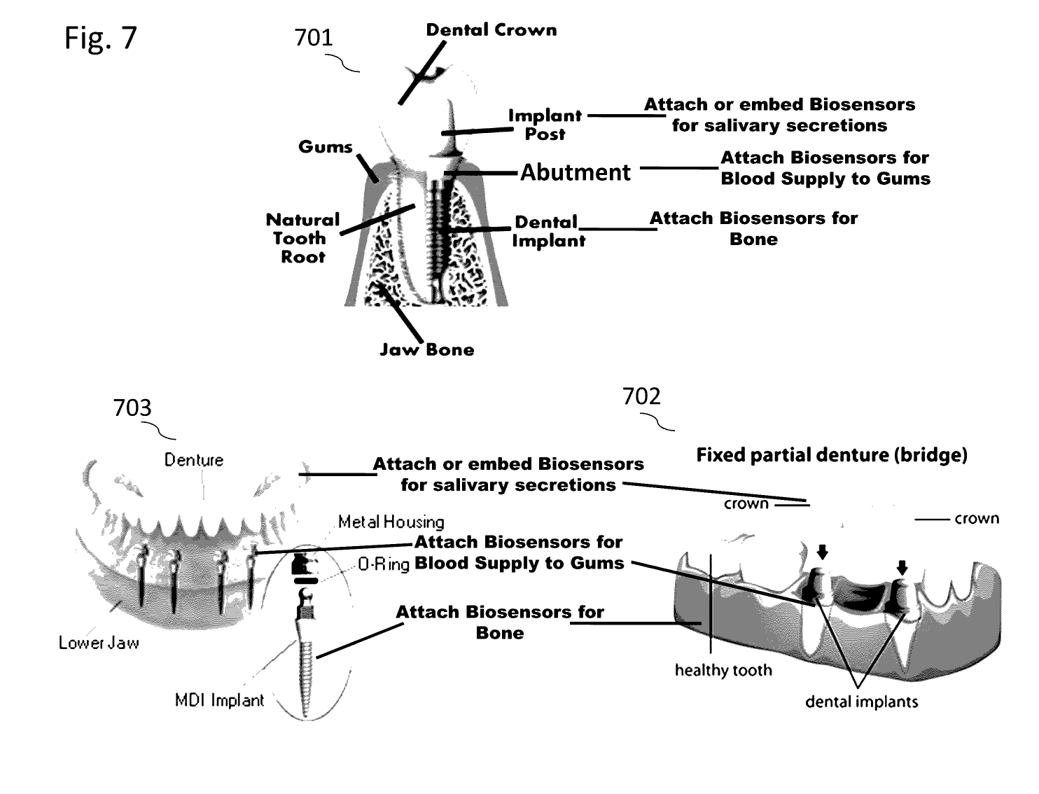

FIG. 7. Fixed or permanent dental devices 701: In certain embodiments, these dental devices are attached to the jaw bone through metal, dental enamel, etc. inserts. Fixed partial denture devices and biosensors (702 and 703) contained or inserted therein may be attached permanently and placed in the oral cavity for several years. This provides a unique opportunity to place biosensors in gums with plenty of blood supply or in jaw bones. Patients with bone disease or who are at risk for bone disease such as osteoporosis will particularly benefit from biosensors that are capable of determining and assessing bone health. Biosensors may also be attached or embedded into these devices so that the biosensors are exposed to secretions in the oral cavity.

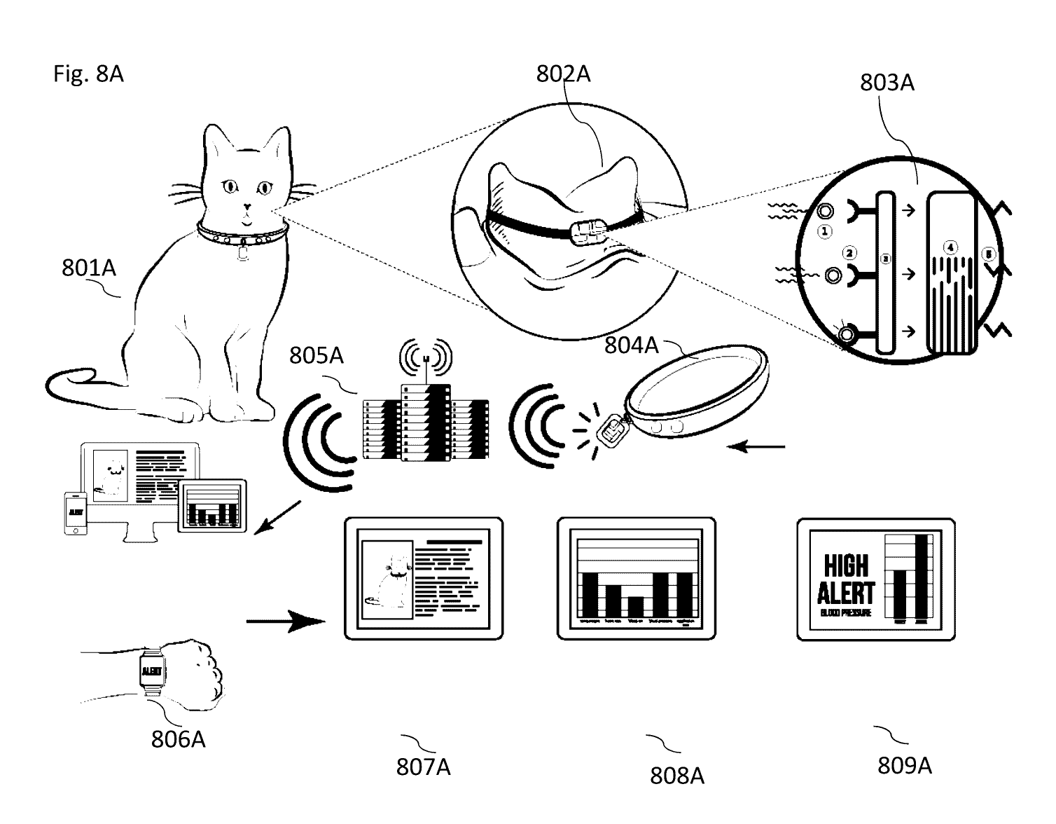

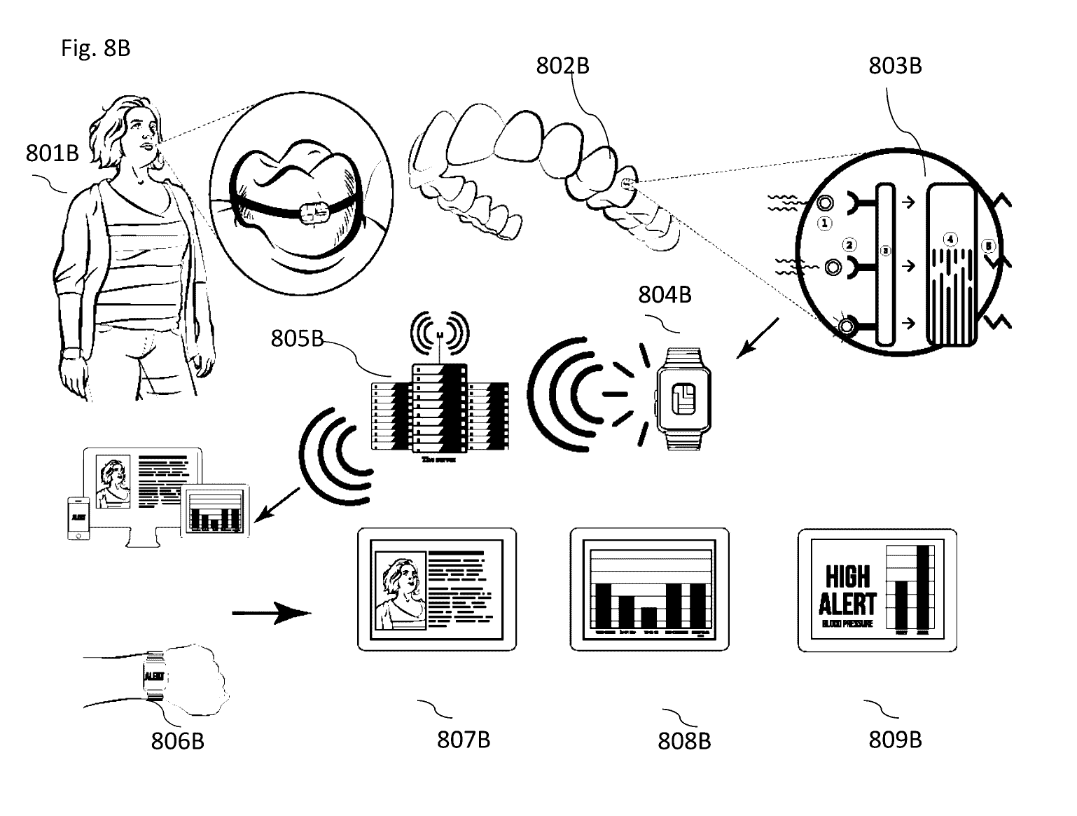

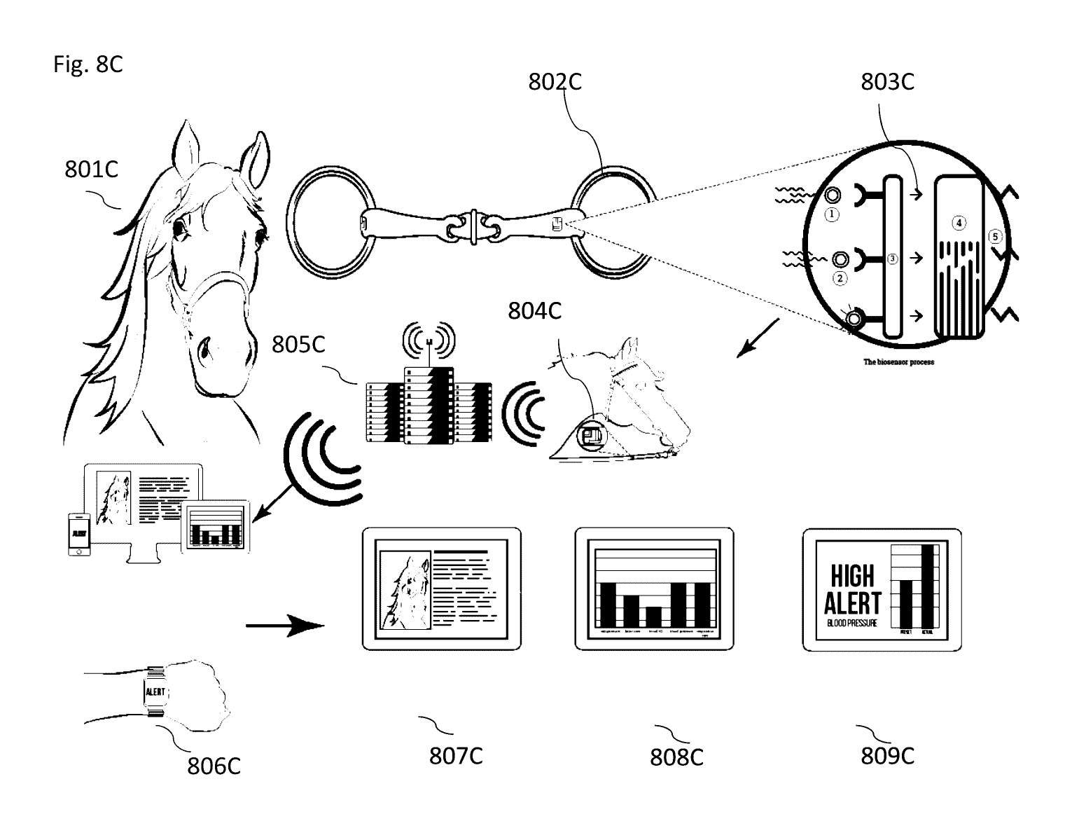

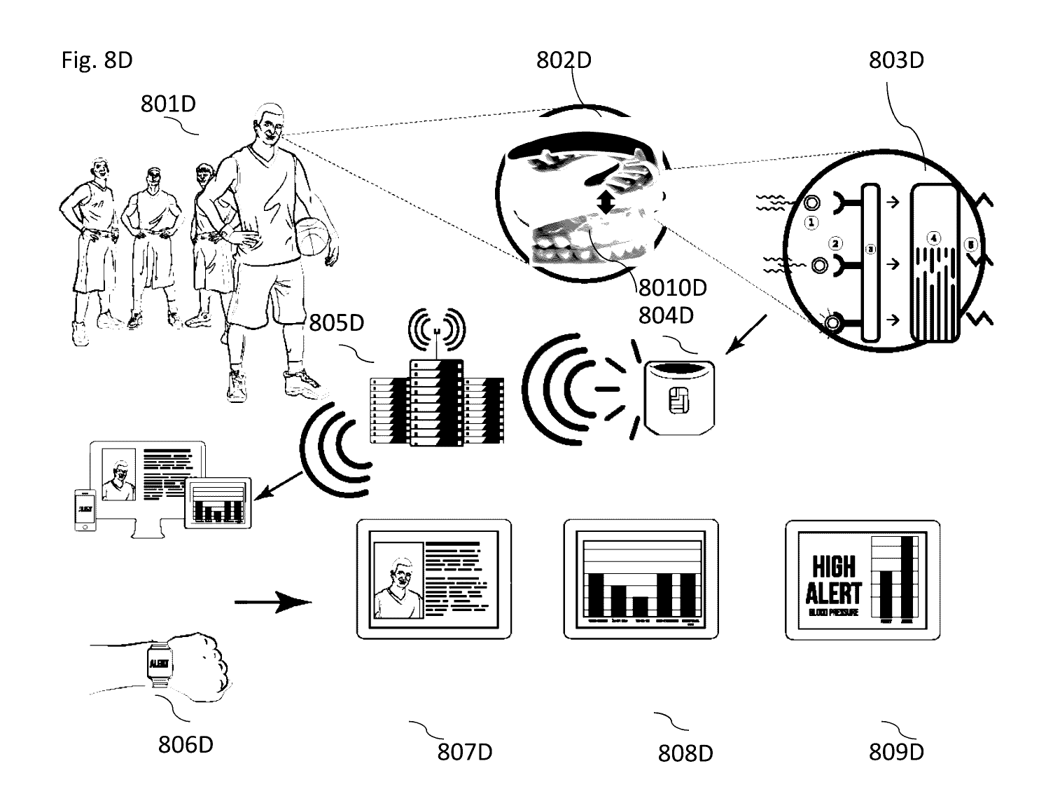

FIG. 8A. In these embodiments of the described OSAC system, a pet or other animal, here exemplified by a cat 801A, has biosensors inserted by attachment to the animal's teeth through a brace, or via another dental device, 802A. The biosensor 803A can be configured to detect any biologic, biologically relevant molecule, temperature, blood pressure, pulse rate, blood oxygen level, respiration rate, accelerometer, gyroscope, blood glucose levels etc. 803A, 1. OSAC devices could collect blood from bleeding due to gum disease, oral trauma and injury, testing, teeth and gum cleansing such as flossing, water pick, blushing, anything that causing or induce bleeding, pin-prick, etc. OSAC could be inserted in the oral cavity to be bathing in the blood to measure blood glucose levels, blood composition, etc. In some cases the sensor could be directed to collect the blood from the bleeding area to detect glucose, blood chemical, medication, etc. And, as needed, the information or signal can then be transduced, amplified, and processed 803A, 2-4. The resulting signal is then transmitted through, e.g., a RFID tag 803A, 5, to a RFID reader on an accessory, collar, tag, clothing, smart phone, or other receiver on or in or around the cat, exemplified here by a smart collar 804A. Alternatively, the oral device is provided as a smart oral device, thereby removing the need for a smart RFID tag reader accessory or device. The signal can then be transmitted to a secure server 805A. Not shown in the figures, the smart collar can also transmit signals from biosensors, nano-particles, etc. on, in, or around the cat. The information can be bi-directionally transmitted through the smart collar to the secure server, which can be through WiFi, Bluetooth, GPS, NFC, or other wireless methods, and in the absence of immediate conductivity, the information can be temporarily stored in the smart device as discussed hereinbelow 805A. The secure server can bi-directionally transmit alerts to pre-selected devices, such as smart phones, iPad, computers, etc. and personnel such as the owner, veterinarian, etc. 806A. The alerts can be transmitted when there are deviations from preset range values put in the system for each biosensor and can also be of varying degrees and tiers. For example, a minor alert can be transmitted for basal readings, and a medium alert can be transmitted if there is an increase, e.g., of 1-2° F. in basal temperature, but for even larger increases in the basal temperature of, e.g., 3-4° F., the alert can be made at a higher intensity, and if major, it can also be, e.g., repetitive requiring acknowledgment of an alert receipt. Not all biosensors need be the same and can generate, e.g., different tiers and degrees of alerts. A small change in heart rate or heart rhythm can generate an alert of higher intensity compared to a similarly small change, e.g., in basal core body temperature or blood pressure. As detailed herein, the biometric data can be viewed in different formats such as, e.g., graphs, histograms, and pie-charts. Various screens can show or verbally narrate alerts and other bidirectional information such as providing different comparatives with other animals of different, same or similar sizes, age, weight, species, etc. or with the cat's or other animal's own previous history; 807-809A.

FIG. 8B. In these embodiments of the described OSAC system, a human, here exemplified by a woman 801B, can have biosensors attached to her teeth through a retainer, or another dental device, 802B. The biosensor 803B can detect, e.g., any biologic, biologically relevant molecule, temperature, blood pressure, pulse rate, blood oxygen level, blood sugar or glucose, respiration rate, or gyroscope, accelerometer 803B, 1. OSAC devices could collect blood from bleeding due to gum disease, oral trauma and injury, testing, teeth and gum cleansing such as flossing, water pick, blushing, anything that causing or induce bleeding, pin-prick, etc. OSAC could be inserted in the oral cavity to be bathing in the blood to measure blood glucose levels, blood composition, medication, etc. As needed, the information or signal can then be transduced, amplified, and processed 803B, 2-4. The resulting signal can be transmitted, e.g., through a RFID tag 803B, 5, to a RFID reader on an accessory, smart jewelry, clothing, watch, other accessories, on, in, or around the woman, exemplified here by a smart watch 804B. The OSAC system can include an RFID tag reader placed within or in proximity to any part of the oral cavity, temporarily or permanently. The signal is then transmitted to a secure server 805B. Not shown in the figures, but detailed herein, a smart watch can also transmit signals from, e.g., biosensors, nano-particles, medical devices, etc. on, in, or around the woman. The information can be bi-directionally transmitted through the smart watch to the secure server which can be through WiFi, Bluetooth, GPS, NFC, or other wireless methods. In the absence of conductivity, the information can also be temporarily stored in the smart device as detailed herein 805B. The secure server can bi-directionally transmit alerts to pre-selected devices, such as smart phones, iPads, computers, etc. and personnel such as the owner, physician, etc. 806A. The alerts can be transmitted when there are deviations from preset range values put in the system for each biosensor and can also be of varying degrees and tiers. For example, a minor alert can be transmitted for basal readings, and a medium alert can be transmitted if there is an increase, e.g., of 1-2° F. in basal temperature, but for even larger increases in the basal temperature of, e.g., 3-4° F., the alert can be made at a higher intensity, and if major, it can also be, e.g., repetitive requiring acknowledgment of an alert receipt. Not all biosensors need be the same and can generate, e.g., different tiers and degrees of alerts. A small change in heart rate or heart rhythm can generate an alert of higher intensity compared to a similarly small change, e.g., in basal core body temperature or blood pressure. As detailed herein, the biometric data can be viewed in different formats such as, e.g., graphs, histograms, and pie-charts. Various screens can show or verbally narrate alerts and other bidirectional information such as providing different comparatives with other animals of different, same or similar sizes, age, weight, species, etc. or with the cat or other animal's own previous history; 807-809A.

FIG. 8C. In these embodiments of the described OSAC system, an equine, exemplified here by a race horse 801C, has biosensors attached to a mouth-bit, bit-guard, bit-gag, lip-strap, or other dental device 802C. The biosensor 803C can detect any biologic, biologically relevant molecule, temperature, blood pressure, pulse rate, blood oxygen level, respiration rate, gyroscope, accelerometer, etc. 803C, 1. OSAC devices could collect blood from bleeding due to gum disease, oral trauma and injury, testing, teeth and gum cleansing such as flossing, water pick, blushing, anything that causing or induce bleeding, pin-prick, etc. OSAC could be inserted in the oral cavity to be bathing in the blood to measure blood glucose levels, blood composition, blood chemical, medication, etc. As needed, the information or signal can then be transduced, amplified, and processed 803C, 2-4. The resulting signal can be transmitted through a RFID tag 803C, 5, to an RFID reader on, e.g., an accessory or other item attached to the horse, including a collar, rein, saddle, or on a horse-rider or jockey, jockey's smart phone, or others, on, in, or around the horse, which could read the biosensors located in the bit when in the horse's mouth, exemplified here by a smart rein 804C. In some situations, biosensors for heart rate, blood oxygen, gyroscope, accelerometer, inertia-sensor, tracking sensors, camera, video, microphone, speakers, etc. could be placed on the horse equipment such as, but not limited to, headstall, headgear, ear-poms, blinker hood, hackamores, noseband, cheese-band, bridle, blinders, winkers, ornaments such as phalerae and sallongs, etc. Various values which integrate the oral bit guard data from the central cardiovascular system could assist in measuring both performance and health through the OSAC systems. Biosensors or cameras could be placed on a blinker hood, nose-piece, or attached to the horse's nose or other facial parts to get more accurate respiration rates. In addition, a heart-monitoring device, heart-rate, or respiration monitoring device can be attached to the saddle or other horse equipment attached to or associated with the horse. Horse heart rate can be monitored by placing biosensors on a manure catcher, or a diaper such that the sensors are under the tail at the tailbone. The heart rate can also be measured by wireless biosensors on horse's leg or other body part. To measure performance, accelometers, gyroscope, inertia-sensors, etc. can be placed at various parts of a horse's body, such as its legs, neck, torso, etc. The OSAC system can thus include an RFID tag reader placed within or in proximity to any part of an oral cavity, temporarily or permanently. Not shown in the figure, but disclosed elsewhere herein, similar to an application for an athlete, the smart horse-rein, e.g., can also communicate a signal from sensors on the horse and other inanimate objects around the horse and from other horses. The signal can then be bi-directionally transmitted to a secure server 805C. The information transmitted through the smart horse-rein, e.g., to the secure server can be through WiFi, Bluetooth, GPS, NFC, or other wireless methods, and in the absence of immediate conductivity, the information can be temporarily stored in the smart device as explained elsewhere herein 805C. The secure server can bi-directionally transmit alerts to pre-selected devices, such as smart phones, iPad, computers, etc. and personnel such as the owner, veterinarian, jockey, or others chosen by the owner 806C. The alerts can be transmitted when there are deviations from preset range values placed in the system for a biosensor and can also be of varying degrees and tiers as aforementioned. Also, as mentioned elsewhere herein, the biometric data can be viewed in different formats such as, e.g., graphs, histograms, or pie-charts. Various screens can show or verbally narrate, e.g., via a talking computer, different information such as different comparatives with other race horses of different, similar or the same sizes, ages, weights, gender, etc. or with the horse's own previous history 807-809C.

FIG. 8D. In these embodiments of the described OSAC system, any athlete, here exemplified by basketball players 801D, can have sensors attached to their teeth, e.g., through an orally inserted device, or any dental device such as a retainer, partial guard, etc. or a combination of an orally inserted device and an accessory device such as a mouth guard, which could be coupled, fitted, attached, etc. to a partial guard or partial retainer, 810D, etc. as exemplified in 802B. The sensor 803B can detect any biologic, biologically relevant molecule, temperature, blood pressure, pulse rate, blood oxygen level, respiration rate, accelerometer, gyroscope, etc. 803D, 1. In some situations, biosensors for heart rate, blood oxygen levels, etc. could be placed on the helmet or other head/face gear because these values from the central cardiovascular system might be required, and these could be measured from the carotid artery or its immediate branches. Biosensors or cameras could be placed on helmet parts or other head/face gear near or on the nose to get more accurate respiration rates. OSAC devices could collect blood from bleeding due to gum disease, oral trauma and injury, testing, teeth and gum cleansing such as flossing, water pick, blushing, anything that causing or induce bleeding, pin-prick, etc. OSAC could be inserted in the oral cavity to be bathing in the blood to measure blood glucose levels, blood composition, blood chemicals, medication, etc. As needed, the information or signal can then be transduced, amplified, and processed 803B, 2-4. The resulting signal can be transmitted through a RFID tag 803D, 5, to an RFID reader on an accessory, helmet, jewelry, wristband, clothing, smart phone, or others on, in or around the player, exemplified here by a smart wrist band 804D. The OSAC system can also include a RFID tag reader placed within or in proximity to any part of an oral cavity. The signal can then be bi-directionally transmitted to a secure server 805B. Not shown in the figures, but discussed herein, the smart wristband can also transmit signals from sensors on other locations on the player, other inanimate objects such as a smart ball, hoop, etc. around the player and also with other players on the team. The information transmitted through the smart wrist band to the secure server can be through WiFi, Bluetooth, GPS, NFC, or other wireless methods, and in the absence of immediate conductivity, the information can be temporarily stored in the smart device as explained elsewhere herein 805D. The secure server can bi-directionally transmit alerts to pre-selected devices, such as smart phones, iPad, computers, etc. and to personnel such as the player, coach, physician, or others chosen by the player, coach, etc. 806D. The alerts can be transmitted when there are deviations from preset range values placed in the system for a biosensor and can also be of varying degrees and tiers as aforementioned. Also, as mentioned elsewhere herein, the biometric data can be viewed for an individual or collectively as a team and can be viewed in different formats such as, e.g., graphs, histograms, or pie-charts. Various screens can show or verbally narrate, e.g., via a talking computer, various information such as different comparatives with other players of a different or the same team, with comparisons made based on different sizes, ages, weights, gender, etc. or with a player or team's own previous history 807-809D.

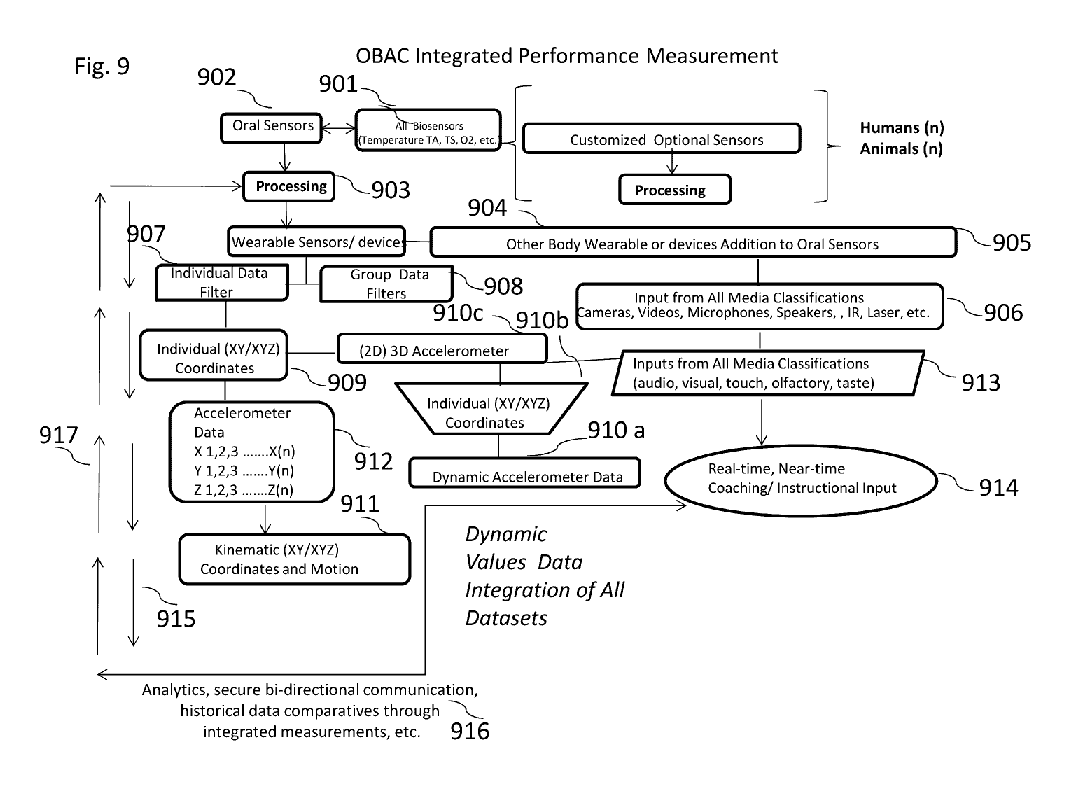

FIG. 9. In these embodiments of the described OSAC system, an example of a fully integrated performance measurement 902 represents all types of sensors which could be standardized or customized and provided as a customizable tool kit for humans, including an infant's smart pacifier and smart feeding bottle, and for animals and integrated through one or more, e.g., accelerometers, gyroscope, is depicted. In 910c, 2D or 3D accelerometer models, which dynamically distinguish both 907 an Individual Data filter, and 908, Group Data filters, of 2D and 3D models, multiple visual sensors, for example, videotaping a sports match to distinguish geometric and mathematical relationships between players, the smart basketball or other ball, smart hoop, smart baseball, smart bat, smart gloves, etc., 904 smart wearable devices worn by athletes and animals on any part of the body (head, upper-back, lower back, legs, knees, shoulder, elbow, hip, ankle, armpit, hand, glasses, contact lens, foot, toe etc. 914 shows real-time or near-time reporting and tracking and also provides comprehensive database and historical data analysis and bi-directional communications for authorized coaches, managers, and teams as exemplified in 917. In 903, advanced computer processing is indicated which can evaluate one or more variables originating from an individual (or animal). 902 oral biosensor and 901 biosensor data such as TA, TS, O2, etc., 905 wearables worn on the body, 906 and input from all media and other sources (temperature, accelerometer, gyroscope, inertia-sensor, tracking, sensors, camera, video, microphone, speakers, video, speakers, IR, thermal, sensors, positioning, laser, gyroscope, etc.), 913 input from all media, classifications (audio, visual, touch, olfactory, taste, etc.), and 910a dynamic accelerometer data 909 athletes position tracking (XY), indoor positioning (XYZ) and all other data sources. The integration and amalgamation of the aforementioned can comprehensively 909 integrate one player's data on a team or 908 multiple players' data on one or more teams in order to integrate the above with 909 positioning, movement and 911 kinematic relationships from multiple modes. The resulting OSAC processed data can utilize probabilistic data association and analytic deterministic data which could help lessen kinematic interference from multiple angles and positions as exemplified in 912. The OSAC system will provide coaches and managers, for example, integrated tools and greater accuracy as to both a player's physical health and energy, but as it relates to precise movements (910b). Since, in sports, 3D situations can be kinematically ambiguous, or at least very difficult from a tracking algorithm standpoint to be accurately established due to, for example, body parts being close together (e.g., an arm may be pressed against, and blend into another player's back, etc.) when videotaping a sports match or training session. The OSAC system collectively provides the coach, trainer or manager 917 secure bi-directional communications, comparatives, historical analysis, time stamped data, reporting and feedback. In addition, the integrated video can be synced with all wearables and other biosensors in order to produce computer-generated precise movement and greater precision and analytics as shown in 916. Individual “wearable” data can be used as part of a team composite calculated from a plurality of wearable “inter” and “intra” devices. Thus, External Structures (ES), Smart Sports Equipment (SSE) and Smart Inter-devices (SIRD), for purposes herein, are devices which can be implanted in the oral cavity, for example. Smart External Wearable Devices (SEWD) are defined herein as devices which can be inter-operationally worn on the body or near the body. External Structures (ES) can be defined by any structure, such as, but not limited to, a playing field, stadium, racetrack, court, including any indoor or outdoor environment, which facilitates an athletic or organizational team. Smart Sports Equipment (“SSE”) is defined as any equipment needed to facilitate their respective sport and the sport's athlete; such as smart-balls, smart-hoops and smart-base-boards and any other device which facilitates their respective sport. Such sports equipment, e.g., smart-balls, can be tracked, their movements traced, mapped and integrated by means known to those skilled in this art. High-definition videos can be constructed or reconstructed when a network of athletes is equipped with smart-wearables, thus helping solve movement ambiguities when integrated and synced with biosensors, wearables, and video. Thus, to increase positive training (e.g., using vibrational, visional or auditory guidance through wearables and other smart accessories for individuals or, collectively, team guidance, and thereby make performance adjustments determined and set by a coach or staff) skills and greatly enhance performance. Players and coaches can use a variety of smart formats and cellular and wireless platforms to communicate with ear pieces and by other means.

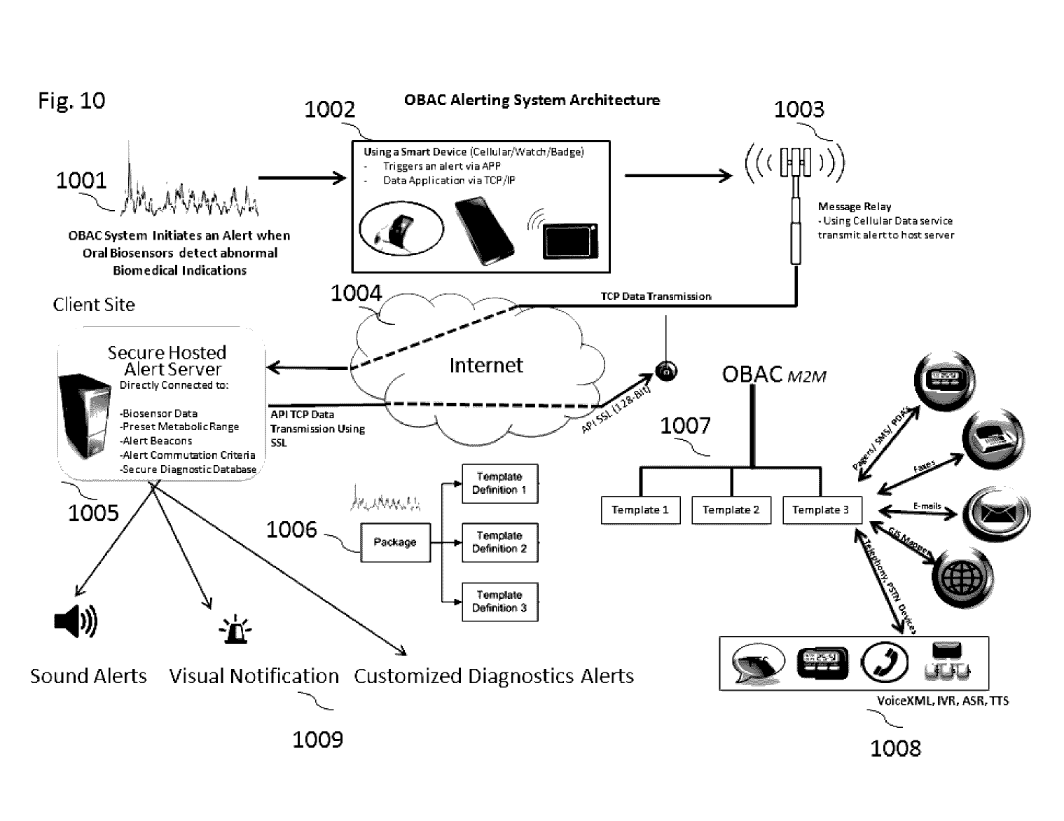

FIG. 10. In these embodiments of the described OSAC system, an example of a fully integrated diagnostic and performance measurement system is provided. 1005 represents a secure host server which can be implemented and utilized by one or more individuals, one or more animals, or one or more organizations. In addition, the present invention can include a privatized internal server host and subsystems as well as one or more external hosted alert servers. A plurality of collective data can be derived from several OSAC oral measurements including, but not limited to, the integration of any type of wearable as described in FIG. 9 and other embodiments in the present invention. A plurality of biosensor data can inform all smart devices 1002, all wearable devices whether smart or not smart, all RFID readers, all can be examined and analyzed in order to determine the degree of an alert (low, medium or high) being dispatched through various templates 1007 referred to today as cloud networks which includes all forms of smart devices, one or more pagers, SMS, Faxes, emails, GIS mappers, beacons (XYZ) telephones, PSTN devices 1008 (Voicemail, IVR, ASR, TTS), satellite phones and other forms of communication. The alert can be dispatched to any computer-aided device or emergency dispatch if the OSAC system detects higher than average or abnormal metabolic ranges, for example. The OSAC system can use one or more templates to help delineate these biometric ranges as exemplified by 1001. 1006 exemplifies the packaging of biosensor parameters as defined (Definition 1, Definition 2, Definition 3 . . . ) by the individual, coach, team and organization etc. In addition, the alerts can be streamed, packeted or stored on the server or on the person(s) or animal(s). Alerts can be represented through preset criteria notification icons converted to SMS, SMS or icons converted to voice alerts, visual notification, touch (vibration) auditory notification and customized through one or more algorithms and diagnostics and secure databases, servers and networks can be used. In addition, bi-directional or multi-directional 1004 API/TCP data, i.e., SSL (128-Bit) data transmissions can use SSL and a message relay using cellular data services 1003 transmitted through one or more host servers. Data application can be the triggering of the alert as previously described, and can be automated (M2M), manual or a combination of both. OSAC alerts can also be combined with APP public general alerts for one or more geographies.

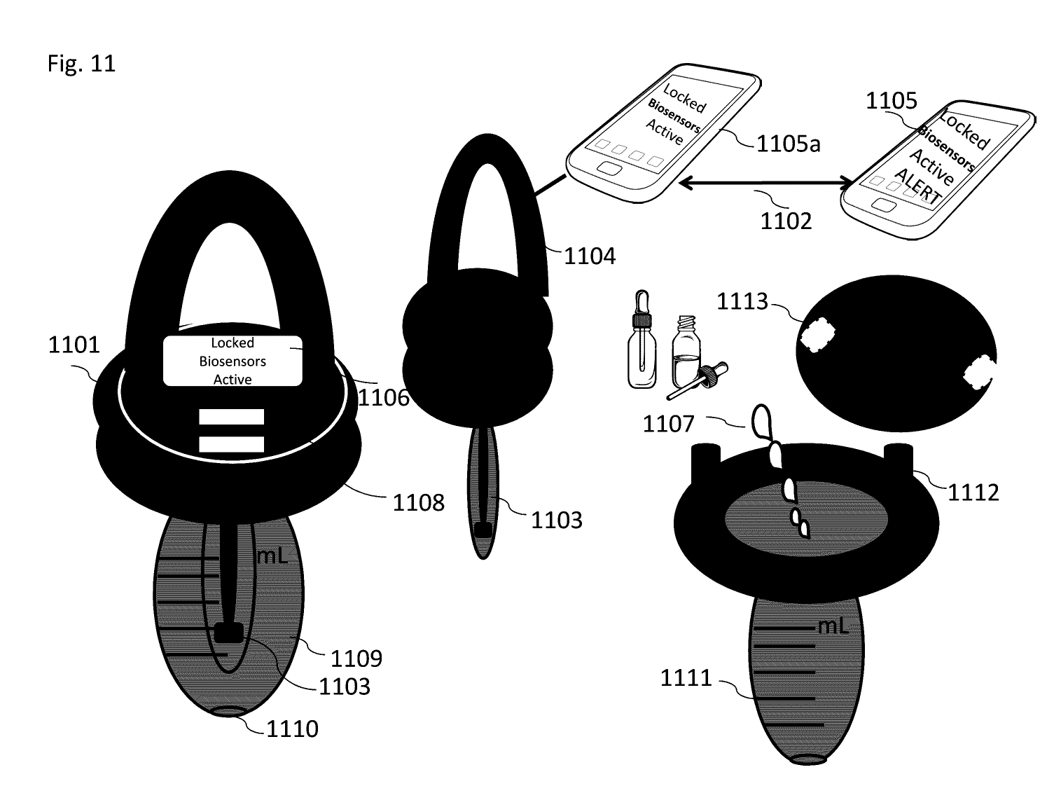

FIG. 11 depicts a smart pacifier 1101 which functions in a plurality of methods, including as an accessory to systems and methods wherein a smart, implantable oral sensor receptacle is also used. Pacifiers are generally nipple-shaped devices which are inserted into an infant's mouth in order to pacify the baby with their natural suckling instinct. The OSAC smart pacifier enables parents and care givers many additional metabolic measurements, for example, embedding one or more sensors 1102, such as temperature, accelerometer, gyroscope, inertia-sensor, tracking, sensors, camera, video, microphone, speakers, video, speakers, IR, thermal, sensors, positioning, etc. within the pacifier's plastic nipple encasement as represented by 1109 or on other parts of the pacifier or pacifier components, for example, a face shield, neck, etc., or on pacifier accessories, such as, but not limited to, a pacifier-strap, strap-buckle, clip, pacifier-holder, clothing, etc. or as a separate attachments. These and other attachments can be configured to be fully or partially removed and attached to clothing, a crib, a toy, a blanket, a baby's person, a feeding-baby bottle, the care-taker, etc. The camera on the accessories can be designed to protect so that it only faces away from the baby and not toward the baby and can be activated and inactivated remotely. This camera can record and send out alerts when anyone, such as a stranger, approaches the baby. Other biosensors or sensors can be integrated with the pacifier, such as a heart monitor attached to the baby's chest or to a baby's clothing on the chest. Biosensor measurements can, for example, be read directly and displayed on the top portion of the pacifier, as represented by 1106 which might also contain a camera, video, microphone, speakers, accelerometer, gyroscope, etc. to monitor and interact with the baby. These can be configured as well to either show data on a screen on the pacifier accessories, pacifier or securely transmit biosensor data through Bluetooth or other wireless means 1104 to one or more smart devices, as represented by 1105 and 1105a. 1105a, e.g., can be the controlling device and software owned by the parents of the child and can monitor and alert the parents of a change in diagnostics, for example, if a child's temperature raises by, e.g., 0.09 degrees, or her pulse becomes abnormally high etc. The parental OSAC device can control what additional devices are notified or sent the child's bio-stats and alerts. In one such example, a child is being taken care of by a day care center or some other party besides the parents, where the 3^{rd} party device, as exemplified by 1105a, is authorized to be unlocked by the parent's host software application. The locking mechanism can disallow such third parties to read biosensor information or can enable the third party caregivers access to the child's diagnostics, for example. Information can be transmitted remotely to one or more devices, which can then optionally share the information. In addition, in another embodiment, a biosensor insert, as represented by 1103, can be interchangeable or securely fixed. The inserted biosensor or sensor can be securely attached to the pacifier but be exposed to content within the oral cavity or it can be configured to be protected by a thin layer of material or for example, an air pocket and outer layer 1109 can be applied to prevent damage resulting from it being chewed by the child, for example. 1104 exemplifies a wireless transceiver chip which is built into the pacifier device enabling the device the ability to send and receive data, commands and other information as represented by 1102. In addition, in yet another embodiment of the invention, the pacifier can be used to dispense both over the counter liquid medication or prescriptions as represented by 1107. 1107 shows a bottle of medicine being poured into the pacifier's calibrated 1111 receptacle and being drawn by the baby when sucking the nipple, thus creating a vacuum. The differential of air pressure will allow the baby to slowly receive the liquid medication at the end of the pacifier insert as represented by 1110. In addition, the pacifier insert can be opened and dismantled by the parents or caregiver to both add the medication to the receptacle as well as wash the device. A secure clamp or other mechanical or electronic (via, e.g., a magnetic mechanism) lock can be used for added safety when fastening or re-fastening. Furthermore, parents and caregivers can remotely control all OSAC smart-phone features, diagnostics, alerting measurements, etc. of such a mechanism through setup software and preference settings.

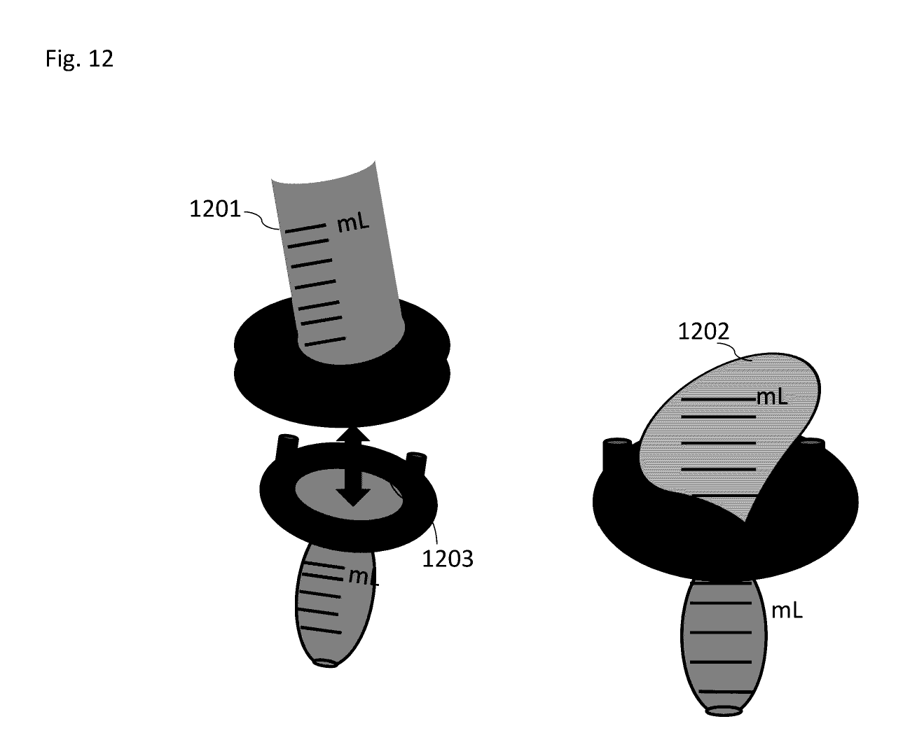

FIG. 12. 1201 represents an embodiment of a pre-calibrated liquid cartridge which can contain one, or a plurality of medications. This plastic cartridge 1201, bag 1202 or other container design can be inserted into the pacifier device. In this embodiment, the clamping mechanism 1203 can either allow insertion of the pre-calibrated medication or release the insert when opened. These pacifier accessories can also be attached to a feeding bottle to dispense and monitor larger amounts of medications, and also to monitor feeding patterns. Feeding time, duration, amount, and liquid temperature may also be monitored using this embodiment of the OSAC system. The OSAC smart pacifier as disclosed herein can utilize numerous measurements and designs using techniques known to those in the “sensing” device arts, which automatically sense when the infant has taken the full dosage, or a partial dosage, and also which automatically and remotely dispense medication as required and/or controlled by the parents.

For an example of a particularly preferred application of the invention, consider that the suffering of animals in laboratory tests, human encroachment on animal terrains, ecological harm and other habitat situations are unforgiving to thousands of species. When attacked by poachers, the “fight-or-flight” response changes their heart rate, respiration rate, and may significantly alter their blood chemistry, so the caretaker and security personnel, if signaled, could intervene to initiate rescue of the animal. Overall health of endangered animals also could be monitored so as to increase their survival and fertility rates and thereby reduce the risk of their extinction.

The oral cavity is a semi-sterile, clean, fast wound healing environment, and has a high threshold for pain because oral secretions include antiseptic-like molecules, wound healing, and pain-killer biologics which are unique to this cavity. In animals, these properties are apparent when they groom themselves by using their tongue to lick away dirt and pathogens, yet no infection results from the licking of pathogen-laden dirt.

In addition, laboratory animals are frequently used for testing various new scientific paradigms, hypotheses, and drugs for the survival or betterment of Homo sapiens that is also cause for animal suffering and even sacrifice. Every year in the U.S. alone, over 25 million animals are used in biomedical experimentation, product and cosmetic testing, and science education. This includes, but is not limited to, dogs, cats, ferrets, rabbits, pigs, sheep, monkeys, chimpanzees, rats, mice, birds, and more. Besides disease status, overall health status of all animals, including test ones, could non-invasively be determined in the oral cavity by the use of the Oral Sensor Alert and Communication System according to the invention. This could reduce the number of animals used, yet increase the quality of animal life and their usefulness.

Table I: List of animals: This table shows some examples of vertebrate animal species that are either pets, farm animals, zoo animals, wild animals, or endangered species or laboratory animals, (Note: some animals could be part of two or more categories).

TABLE I

| | | | Zoo & | Critically | |
|---|---|---|---|---|---|
| | | Farm | Wild | Endangered | Laboratory |
| Species | Pets | Animals | Animals | Animals | Animals |
| Fish | Tiger Barb, | salmon, | Lionfish, | Yangtze Finless | Zebrafish |
| | goldfish | tilapia, | Peacock Bass, | Porpoise, | |
| | | catfish | Black & Ray | Vaquita | |
| | | | Pacu | | |
| Reptile | gecko, turtles | alligators, | alligators, | Leatherback, | African House |
| | | crocodiles | crocodiles, | Madagascar Big- | Snakes, lizards |
| | | | lizards, snakes, | headed, and | |
| | | | turtles, | Hawksbill Turtle | |
| | | | tortoises, | | |
| | | | salamander | | |
| Amphibians | frogs | frogs | frogs, toads, | Yosemite toad, | frogs, toads, |
| | | | newts | Chinhai | newts |
| | | | | salamander, | |
| | | | | Baracoa dwarf | |
| | | | | frog | |
| Avian | parrot, ferret | chickens, | flamingo, | Giant Ibis, New | chickens |
| | | turkey, duck | hummingbird, | Caledonian | |
| | | | mockingbird, | Owlet-nightjar, | |
| | | | cranes | California | |
| | | | | Condor, kakapo, | |
| | | | | California | |
| | | | | Condor | |
| Mammalians | rabbit, guinea | bison, cattle, | tiger, | Amur Leopard, | rats, rabbit, |
| | pig, dog, cat, | deer, goat, | elephants, | Black, Javan and | guinea, mice, |
| | hamster, horse | horse, mule, | hippopotamus, | Sumatran Rhino, | pig, dogs, |
| | | pig, | cheetah, polar | Cross River, | hamster |
| | | reindeer, | bear, panda, | Mountain and | |
| | | sheep, | and seals | Western | |
| | | buffalo, yak | | Lowland Gorilla, | |
| | | | | Pangolin, Saola, | |
| | | | | South China and | |
| | | | | Sumatran Tiger, | |
| | | | | Sumatran | |
| | | | | Elephant, | |
| | | | | Sumatran | |
| | | | | Orangutan | |

Because animals (Table I) are unable to communicate about their health, an OSAC system, as described, becomes an important part of their healthcare management. Most farm and wild animals have no daily contact with humans, so these systems are able to provide very useful information about their health status and well-being. Medical intervention is allowed to occur on an “as needed” basis as information is gathered. Animals flagged for possible attention, may be further diagnosed, isolated from the rest of the herd to prevent further spread of diseases, and infections, and targeted for subsequent treatments. The herein described OSAC system permits critically endangered animals to benefit from these sensors and biosensors because their health status can be monitored and intervention provided in real time and preemptively to save their lives and help in their propagation.

In humans, oral fluid originates mainly from secretions of parotid, sublingual, and sub-mandibular glands, and a large number of minor salivary glands. Non-glandular constituents of salivary fluids originated from esophageal mucosa, food debris, crevicular fluid, blood-derived compounds, infections, and many other oral bio-sensate molecules could potentially be used in diagnosing health and disease status. In addition, the collection and evaluation of secretions from individual salivary glands are used for the detection of gland-specific pathology such as infection and obstruction. The Academy of General Dentistry suggests that more than 90 percent of all systemic diseases, diseases that affect or pertain to the entire body and not just one of its parts, produce oral symptoms and are reflected in oral secretions. Thus, the oral cavity serves as a critical vantage point for detecting the early onset, signs, and symptoms of diseases including, but not limited to, systemic infections like AIDS, cardiovascular diseases, atherosclerotic inflammation, and stroke, preterm delivery with low-birth weight babies, dental diseases, and tooth decay. It is a site for oral cavity infections that damage teeth and gums. In the case of humans, the elderly, babies, and the disabled who need constant monitoring but are unable to communicate their health status could also benefit from real time detection of such biosensors. The “Disposable Medical Devices Sensors Market” includes but is not limited to biosensor, image sensor, accelerometer, gyroscope, monitoring smart pill, pulse, oximeter, diagnostic capsule endoscope, blood glucose strip, therapeutic insulin pump, and diagnostic global forecast. However, whole saliva systems are not available for real time study and evaluation of systemic disorders, and there is a lack of an effective means for real-time or near-time diagnosis.

Maintaining core basal body temperature in humans and other warm-blood animals, mammalian and avian, is paramount for optimum health. Any deviation in basal or at rest core body temperature for warm blooded animals is indicative of disease such as infections, inflammation, etc. Core body temperature can be most accurately measured through three places on human or animal body, the inner ear, the oral cavity, and the rectum. An OSAC system with a temperature sensor, according to the invention, can serve as an alerting thermometer. An ear piece outfitted with an inner ear thermometer becomes an ear alerting thermometer and ear disease detecting and alerting device. The ear piece can also be used to alert and measure heart rate and blood pressure, etc.

These aforementioned commonalities are true for animals, including, but not limited to, pet, farm, zoo, wild, equine, and laboratory animals (Table I), as it is true for human and related animal diseases as shown in (and see some examples of, in Table II):

TABLE II
Disease

| | |
|---|---|
| | Biologically derived Biomarkers |
| Cytomegalovirus infection | Nucleic Acid |
| Dengue viral fever | IgA (immunoglobulin), Elevated Body Temperature |
| Ebola virus infection | IgG (immunoglobulin), Nucleic Acid, and Elevated Core Body |
| | Temperature |
| Multiple Sclerosis | IgA (immunoglobulin) |
| Epstein-Barr virus infection | Nucleic Acid and Proteins |
| Herpes simplex viral infection | Proteins, and Nucleic Acid |
| Acquired Immune Deficiency | IgG, (immunoglobulin), Nucleic acid, Blood Chemical, Microbes, |
| Syndrome | and Protein |
| Hepatitis A viral infection | IgM, IgA, IgG, (immunoglobulin), Proteins, Microbes, Nucleic Acid |
| Hepatitis B viral infection | HbsAg, HbsAb, HbcAb, (immunoglobulin), Proteins, Microbes, and |
| | Nucleic Acid |
| Hepatitis C viral infection | IgG, (immunoglobulin), Proteins, Microbes, and Nucleic Acid |
| Human herpesvirus infection | Nucleic Acid, Proteins, Microbes, and IgG |
| Malaria, Plasmodium falciparum | IgG (immunoglobulin), Elevated Core Body Temperature, Microbes, |
| infection | Nucleic acids, Proteins |
| Periodontal disease | Protein, Microbes, Gases, and Nucleic acid |
| Oral and Lung Cancer | Protein, Microbes, Gases, and Nucleic acid |
| Mammary & Pancreas Gland | Protein, Microbes, Gases, and Nucleic acid |
| Carcinoma | |
| Mycobacterium tuberculosis | DNA (Nucleic Acid), Elevated Core Body Temperature, Protein, |
| infection | Microbes, Gases, Nucleic acid |
| Age Related Diseases | Methylated DNA (modified Nucleic Acid), Nucleic Acids, Proteins, |
| | Modified Proteins, Blood Chemicals, Gait, Movement, |
| | Immunoglobin |
| Sjogren's syndrome | Nucleic Acid and Protein |
| Oxydative stress | Protein and Nucleic Acid |
| Inflammation | Protein, Elevated Temperature, Microbes, Nucleic Acid |
| Malnutrition | Proteins, Cations, Anions, Micronutrients, Vitamins, Minerals |
| Allergies | IgG (immunoglobulin), Allergens, Microbes, Nucleic Acid |
| Genetic Disorders, Cystic | Nucleic Acid, Proteins, Salts, Cations, and Anions like K⁺, Cl⁻, NaCl, |
| Fibrosis | Ca²⁺ |
| Blood Disorders | Immunoglobulins, Proteins, DNA, Microbes, Blood Chemical, and |
| | Nucleic Acids |
| Cardiovascular and Circulatory | Proteins, Immunoglobulins, Microbes, Blood Chemical, Blood |
| Diseases | Pressure, and Nucleic Acids |
| Renal Disease and Urinary | Proteins, Nucleic Acid, Salts, Cations, Anions, Minerals, Ammonia |
| system Diseases | Containing Gases, and Blood Chemicals |
| Osteoporosis, Skeletal system | Proteins, Minerals, Salts, Nucleic Acids, Electrical Waves, Blood |
| diseases, and Muscular diseases | Chemicals, Immunoglobins, and Microbes |
| Integumentary system diseases | Proteins, RNA, DNA, Electrical Waves, Salts, Blood Chemicals, |
| | Immunoglobins, Blood Chemical and Pathogens |
| Fatigue | Proteins, Lactose, Metabolites, Nucleic Acids, and blood Chemicals |
| Stress | Cortisol, Immunoglobulins, Proteins, Microbes, Blood Chemical, |
| | and Nucleic Acids |
| Taste, Ageusia, Hypogeusia, | Neuro-peptides, Neuro-chemical, Nerve function, Electrical |
| Dysgeusia | Properties, Gases, Microbes, Protein, Nucleic Acids, Chemicals |
| Chewing, Mastication | Movement, Proteins and Digestive Enzymes, Pressure, DNA, and |
| | RNA |
| Bad or Fruity Breath | Neuro-peptides, Neuro-chemical, Nerve function, Electrical |
| | Properties, Gases, Microbes, Protein, Nucleic Acids, Chemical |
| Digestive System, Liver, Spleen, | Proteins, RNA, DNA, Enzymes, Electrical Waves, Blood Chemical, |
| Gall-bladder, Pancreas | Chemicals, Immunoglobins, Pigments, Acids, Salts, Anions, Cations, |
| | PH, and Pathogens |
| Endocrine, Lymphatic, and | Proteins, RNA, DNA, Electrical Waves, Chemicals, Immunoglobins, |
| Excretory Diseases | Blood Chemical, Pathogens, Salts, Hormones, and Lymph Fluids |
| Sleep Deprivation | Alpha-Amylase, Sleep Patterns, Motion, Neuro-peptides, Neuro- |
| | Chemical, Nerve function, Electrical Properties, Gases, Protein, |
| | Nucleic Acids, Chemicals, and Breathing |
| Stroke | Muscle tone, Electromyography (EMG), Proteins, and Nucleic acid |
| Fertility, Ovulation, | Proteins, Hormones, Nucleic Acids, and Core Body Temperature |
| Reproductive Diseases | |
| Depression, Alzheimer's Disease, | Proteins, RNA, DNA, Electrical Waves, Chemicals, Immunoglobins, |
| Neurological Disorders, | Blood Chemical, Neuro-Peptides, Neuro-Chemical, Nerve function, |
| Dementia and other Neuro- | Electrical Properties, Gases, Microbes, Nucleic Acids, Chemicals and |
| Diseases | Pathogens |
| Psychology, Mental Disorders | Proteins, RNA, DNA, Electrical Waves, Chemicals, Immunoglobins, |
| and Mentally Challenged | Blood Chemical, Neuro-Peptides, Neuro-Chemical, Nerve function, |
| | Electrical Properties, Gases, Microbes, Nucleic Acids, Chemicals, |
| | Gait, Tracking, Movement, Behavior, and Behavior-Patterns |
| Nervous system diseases | Proteins, RNA, DNA, Electrical Waves, Chemicals, Immunoglobins, |
| | Blood Chemical, Neuro-Peptides, Neuro-Chemicals, and Pathogens |
| Diabetes | Sugar, Acetone, Proteins such as hba1c, Proteins, Nucleic acids, and |
| | Blood Chemical |
| Dehydration | Salts, Cation, and Anion Concentration, Humidity or Water |
| | Concentration, Proteins, and Nucleic Acids |
| Halitosis | Sulphur Containing Gases, Microbes, Enzymes, Nucleic Acids, and |
| | Proteins |
| Respiratory and Pulmonary | Immunoglobin, Salts, Water Concentration, Nucleic Acids, Proteins, |
| Disease | Temperature, Blood Chemicals, Gases, Lung Capacity, and Muscle |
| | Tone |
| Ear, Nose, Throat Diseases | Temperature, Pulse, Blood Chemicals, Proteins, DNA, RNA, |
| | Electrolytes, Movement, Gait, Gases |
| Infant and children diseases | Temperature, pulse, blood chemicals, proteins, DNA, RNA, |
| | Electrolytes, Movement, Gait, Gases |
| Alcoholism | Alcohol, Proteins, Enzymes, and Nucleic Acids |
| Pollution | Chemicals, Gases, Proteins, Nucleic Acids, Microbes, and Pollutants |
| Drug addiction | Drug molecules, Proteins, and Nucleic Acids |
| Allergies | Allergenic Molecules, Immunoglobins, Nucleic Acid, and Proteins |
| Hypoxia | Decreased Oxygen Concentration, Blood Chemicals, Proteins, Nucleic |
| | Acid, Gases, and Pressure |
| Toxins | Gases, liquids, Solids, Proteins, Blood Chemicals, and Nucleic Acids |
| Smoking | Nicotine, Proteins, and Nucleic Acids |
| Gait | Accelerometer, Gyroscope, Inertia Sensors, Nucleic Acids, Proteins, |
| | and Blood Chemicals |
| Obesity | Selenomonasnoxia, Proteins, Nucleic Acids, and Blood Chemical |
| | Microbial Biosensors |
| Cancer, oral | Capnocytophagagingivalis, Prevotellamelaninogenica, Streptococcus |
| Cancer, Pancreatic | Neisseria elongata, Streptococcus mitis |
| Crohn's disease | Fusobacteria, Firmicutes |
| Chronic pancreatitis | Granulicatellaadiacens, Streptococcus mitis |
| Periodontal disease | Aggregatibacteractinomycetemcomitans, Porphyromonasgingivalis, |
| | Prevotella intermedia, Tannerella forsythia Campylobacter rectus |
| Dental Caries | Streptococcus mutans, Lactobacillus |
| Obesity | Selenomonasnoxia, proteins, nucleic acids, blood chemical |
| Other (non-disease) for criminals, | Tracking, Movement, Gait, Performance, Behavior, Behavior- |
| athletes, disabled, elderly, | Patterns, Feeding, Vitals Patterns, Remote Patient Monitoring, |
| infants, children, race-horse, pets, | Location, Food Components, Ingestion Pattern and Monitoring. |
| other animals, etc. | |

Unlike any other body cavities, the oral cavity represents a unique cavity because it is easily accessible, contains taste buds and lymph nodes, is highly vascularized and enervated, has a very absorptive surface, and secretes several salts, solutes, solvents, biologicals and biomarkers such as proteins, enzymes, nucleic acids, immunoglobulins, and other pertinent biological measurements. In addition, salivary secretions also contain fast healing and analgesic properties. These properties are experienced by most people during their daily teeth cleaning routine that often causes painless and fast healing gum bleeding. This example demonstrates the naturally-occurring aseptic, pathogen-destroying, and natural healing properties of saliva. Compared to currently used body parts, such as skin (which in animals adds extra complications due to the presence of fur) used to place or implant biosensors, oral cavity biosensors are less painful, heal fast and are more comfortable since any broken tissue during sensor placement or insertion is fast healing, has less of a possibility of infection, and in most cases, would involve a one-time wound. These qualities make oral biosensors and other sensors less invasive or minimally invasive. Discrete without being visible and yet easily accessible, they can be worn round the clock, including during rest, sleep, high activity, and more. Additionally, they are readily calibrated, rejuvenated, and/or cleaned. An increase in patient, both human and animal, compliance would result from their non-invasive or minimally invasive nature and ease of accessibility. Further, a prolonged lifespan for these sensors is possible due to their protection from external elements and placement in a relatively aseptic environment. This prolonged lifespan is demonstrated by dental devices that can frequently last several years. Thus, these oral biosensors, used in accordance with the invention, reduce healthcare cost while simultaneously increasing patient compliance. Oral biosensors and sensors used as described herein are superior to other biosensors in that they are able to measure biologics and biomarkers intermittently, frequently, or constantly as needed to treat and manage most diseases. Once the biosensor is placed in the oral cavity, it can monitor and collect data for seconds, minutes, days, months, or even years. These biosensors may be long-term or short-term, permanent or temporary, unlike other diagnostic testing methods. Non-oral biosensors that are attached to other body parts typically remain attached just a few hours. Just like a bandage typically stays on the skin for but a few hours to a couple of days, but a dental brace typically lasts for weeks, months or years.

Cheaper and personalized biosensors or sensors of the invention become important in managing healthcare costs and outcomes for multiple diseases. As technologies such as nano-technology or 3D printing become more advanced and easily and cheaply available, the OSAC system of the invention may be adapted to provide additional savings in healthcare costs for both humans and animals. Furthermore, the present invention enables biosensors or sensors to be uniquely communicated from one or more animals and humans to a plurality of OSAC systems; thus, adding to the utility of the sensors. The OSAC system of the invention enables humans to monitor and alert themselves or others, and enables animal-OSAC units to send alerts to humans in order to monitor and alert their caretakers. The present invention systematically integrates all forms of oral based sensors in order to offer new, innovative, and unique opportunities to monitor the overall health status of both humans and animals. They also help to preemptively and accurately diagnose, detect, and monitor, and thus, help in the prevention, prognosis and risk-assessment of a variety of diseases. The oral cavity is a window into the overall health and disease status for the entire body and the OSAC system of the invention enables and integrates a multitude of the aforementioned devices to quickly and efficiently communicate a person or animal's heath landscape and schematic map. When blood drawn from a patient is sent to a laboratory for diagnosis, this involves an invasive, painful, costly, and time consuming process with frequent delays of usually days before diagnostic results become available to healthcare providers and patients. The present invention describes a needed precursor to, or substitution for, other more invasive methods of health and disease diagnoses. Patient non-compliance results from the painful blood draw which too often causes infections. Many chronic or frequent measurements simply would not need to be performed when substituted for with OSAC monitoring, which makes treatment more likely. Cost of healthcare increases enormously due to untimely and delayed diagnoses that interfere with proper treatment and good prognoses. Early and timely diagnoses for many diseases including, but not limited to, cancer, diabetes, cardiovascular diseases, and other metabolic diseases, could result in better prognoses, improved quality and quantity of life, and decreased healthcare costs.

The OSAC system of the invention is uniquely developed to provide highly customizable information and alerts. This is in part due to the large and highly variable number of sensors, sensor receptacles, accessory receptacles, network interfaces and network components that may be utilized and combined.

Accordingly, in a particularly advantageous set of embodiments, the invention offers an OSAC customizable developers' or development tool kit or platform which provides people, companies, organizations, hospitals, teams, institutes, or others to create their own OSAC system and employ OSAC methods which are custom-tailored to meet their specific needs and optimum circumstances. For example, using the OSAC developers' customizable tool kit or platform, the needs of patients, sports teams, and organizations can utilize a system for one or more settings, ranges, dashboards, historical or other data graphs, biometrics, analyses, and/or for their own market-base. Differing temperature alerts may be monitored when a pre-established range is adjusted based on customized datasets. The devices of the OSAC system can be wireless and completely contained in the oral cavity, or may use accessory sensors (or external ones). The OSAC tool kit and software may be customized by physicians, coaches, etc. to e.g., monitor vitals, including but not limited to temperature for patients, athletes, pets, other animals, etc. Alerts, as described elsewhere herein can be triggered remotely to read periodically on an as needed basis, and the data stored on a chip or system, and then streamed, or directly streamed and transmitted. The OSAC system can be programmed to periodically read the temperature and other sensor-derived information through a present alerting mechanism. Such tool kits may be devised where they periodically trigger the measurement of such readings when needed in any time increment or when readings are optimum to measure health or performance. Moreover, the customizable kits can be optimized to be integrated with other sensors, accessory or alternative wearables, as well as a mix and match of the various system components and method steps described in detail herein.

A feature of many embodiments of the OSAC system and methods of the invention is the dynamic nature of the integrative alert components that may be built into the customizable operations. For example, people, individuals, groups, teams, hospitals, organizations, etc., may be alerted on a dynamic basis when one or more or variable combinations of, program range settings of various parameters are exceeded. The customizable alert modes may be operated as real-time or near-time slumber to alert, programmed, automatic, manual control, geographic-specific, disease or condition-specific, gender-specific, age-specific, species-specific, situation-specific, activation-triggered, or woken-up by another sensor, device or remote mode, or any combination of such customized parameters and components. Illustrative of this, is the complex integrated system depicted by FIG. 9. This flow chart of an integrated OSAC system includes one or more of movement, audio, video, pressure, impact, sensory wearables, etc. which provide kinematics and other feedback to enhance accurate measurement of performance, movement, biometrics, and can add in health-related and physiological inputs as well to optimize health, well-being and/or performance criteria.

The OSAC system of the invention in one embodiment, for example, measures respiration rate, heart rate, blood pressure, temperature, blood O₂ levels, inflammation, etc. as a component of the “practiced” oral device insert. A “practiced” oral device insert may include any customized device inserted in an animal or humans' oral cavity and is designed to monitor any metabolic situation such as, e.g., those described in Table II. OSAC as used in the invention includes mobile device interfaces, which read a variety of diverse sensors. Moreover, with the discovery of new biomarkers, oral biosensors of applicant's invention are even more useful in detecting, diagnosing, risk-assessing, prognoses, and monitoring even more diseases. 3D printing applied to the invention makes cost-effective and less costly custom dental devices available and thus makes our OSAC system more cost-effective. In one embodiment, the present invention involves pre-required diagnoses for treatment of a disease. Diagnostic testing is performed based on a patient's symptoms and history. Patient communication is one key element to gathering information about a patient's symptoms and history. Animals are unable to communicate their symptoms, so simple diagnostic tests such as frequent measurements of heart and respiration rates, temperature, and blood pressure are utilized in determining their health and disease status. However, such diagnostic testing in many animals requires them to be sedated. Using one-time sedation to place or implant an OSAC system of the invention, these oral biosensors offer a unique and novel opportunity to measure constantly or frequently as needed for these diagnostic purposes. The elderly, disabled, mentally ill, babies, and chronically ill could similarly benefit from frequent measurements. Conventional methods are limited by time and location of routine diagnoses like temperature, blood pressure, or blood chemistry, etc. However, the biosensor system of the present invention offers a flexible and unique opportunity to measure these statistics at home, at work, during exercise, or even while sleeping. The device may be present in the oral cavity for a short (few seconds to minutes to few hours) or long time (several days to months to years), and may be temporary or permanent. The OSAC system can be applied and may be part of a plurality of dental devices including, but not limited to, caps, crowns, bridges, mouth-guards, denture, implants, veneers, fillings, fixed prosthesis, braces, and/or wires, and retainers, mouth-guards, horse-bit, occlusal splints and/or temporary/removable materials, used in dentistry and recreationally (tongue piercing, etc.). OSAC system could also be applied to devices that are partially inside the oral cavity and partially out-side the oral cavity such as pacifiers, smart thermometers, a smart gauge, smart dipstick, smart rod, smart stick, smart device to collect blood or saliva, etc. A plurality of biosensors and RFID, micro- and nano-communication devices, micro- and nano-enabled energy harvesting, micro- and nano-energy storage, micro- and nano-devices or components may be installed in any dental device and/or placed in any oral cavity by means known to one skilled in such arts, depending upon the medical or diagnostic intent.

In yet another embodiment, the OSAC systems of the invention may be used in conjunction with other smart wearables or attachables on one or more individuals creating a measurable team diagnosis. Furthermore, smart wearables or other medical devices may be utilized by a collective group, including in any team sport application, i.e., basketball, soccer, baseball, hockey, swimming, track, football, cricket, gymnastics, and other Olympic or global sports, etc. It is understood that the OSAC system of the invention may measure and diagnose individual performances in sports (singularly measured, analyzed and diagnosed) or these may be measured, analyzed and diagnosed, collectively as a team composed of individual players. The device can be implanted, flexible, adhered, fixed, and/or cemented to the floor of the oral cavity, inside of the cheeks, tongue, and the sublingual cavity and/or affixed in other places within the oral cavity. The device locations within the oral cavity are optionally chosen by the patient or his/her caretaker and/or healthcare provider. The choice of location is one based upon a combination of factors including, but not limited to, convenience, comfort, durability, exposure duration to molecules to be measured, and duration of the device placement in the oral cavity. The sensor may be placed by a healthcare provider including, but not limited to, physicians, surgeons, dentists, veterinarians, or healthcare provider assistants, caretakers, and/or patients themselves. The placement is usually made with the assistance of one of the following; for example: mild sedation, full sedation, local anesthesia, general anesthesia, or other. Dental devices represent biologically inert surfaces; whereas, other areas of the oral cavity such as the gums or tongue, are biologically active and non-inert. The sensors therefore may be placed fully on inert surfaces, biological active surfaces and/or a combination of both. The sensors may be exposed to elements of the oral cavity or covered with a barrier or a combination of both.

OSAC materials inserted in the oral cavity as described in the present invention may detach, clip, be alerting chips, etc., and can be constructed for a short time period (temporarily) or constructed for a long-term (e.g., permanent) time period. All time periods for the plurality of orally inserted and attached devices used for one or more functions of the invention may be deployed. Oral devices employed include, but are not limited to, biosensors, RFID tags, and any inserted attached or detachable dental device used for humans and animals to obtain data and sensor-derived information. The design and construction of the inserted oral device will vary for each species and is customizable to detect, monitor and/or alert for all types of medical conditions.

In addition, in another embodiment of the present invention, the design of the OSAC system is configured to help avoid the possibility of choking, for example; made of smooth (or partially or fully encapsulated or encased, oval, round, rounded or in any shape or size of similar utility) which could easily be swallowed and digested or passed through the digestive process; the biosensor itself might not be smooth. OSAC materials used herein are optionally made un-chewable or chewable depending on the species, medical need, and functionality and durability requirements. In addition, an alert can be sent when the sensor or OSAC device becomes semi and/or completely detached or travels to other parts of the body, such as the esophagus or stomach. The OSAC material employed may be designed to dissolve or break apart in order to prevent choking which could be particularly useful in the case of animals, children, the disabled, and the elderly, and others who cannot communicate that the device was detached and swallowed.

In addition to oral sensors and biosensors, the OSAC system of the invention includes sensors, biosensors, and nano-particles located elsewhere within and/or on an animal or human that interfaces with the OSAC system. One example of this is a smart pill that releases a biosensor and nano-particles in the animal's stomach which interfaces with the OSAC system to activate, e.g., an OSAC blood pressure oral biosensor. Motion sensors in a room might be used to determine the gait of a patient. These sensors may also interface with oral biosensors to determine the cause of a patient's poor gait, which can be, e.g., elevated temperature, blood pressure, altered drug concentration, etc. Accurate information regarding the cause of the patient's poor gait would help in preventing accidents resulting from his or her poor gait. The OSAC system also can interface with various other devices located on or near an animal or human and securely communicate with a plurality of remote devices monitoring the health and/or well-being of one or more animals or humans. These devices are optionally within a human or animal body, e.g., such as, a drug dispensing pump or other. Alternatively, the device may be located on the human or animal's body, such as a cardiac monitor, dog collar, and accessories or other. These devices may read and interface with a variety of diverse biosensors such as blood pressure and heart rate components of the OSAC system with a cardiac monitor and a drug dispersing pump as one example. The OSAC system may also communicate with nano-particles in any body part.

In a further embodiment, OSAC includes a plurality of wireless local area network (LAN), wide area network (WAN) controllers providing system-wide wireless local area network (WLAN) functions such as intrusion prevention, Radio frequency (RF) management, encryption, communication integrity, quality of service (QoS) and mobility are included within the scope of the current invention. Additionally, one or more databases, routers, secure servers, controllers, switches, etc., may be utilized when transmitting oral sensor data through WAN/Internet configurations and multiple, independent databases are manually or dynamically combined for specific applications. In yet another aspect, a combination of one or more elements of metabolic alerts are combined with one or more mobile and/or wearable devices to observe, monitor, measure and/or alert any particular metabolic situation. The OSAC system of the invention also customizes each alert in order to tailor the alert and/or response to the specific needs and to help prevent the loss inputs and alerting that may transpire in real-time or near-time, depending on the severity, sources, number of inputs and need.

The communication equipment connects to a main communication system of the cellular phone system through a variety of means, including, but not limited to, a Mobile Telephone Switching Office (MTSO) or a Mobile Switching Center (MSC). The phrase “public Land Mobile Network (PLMN)” will be used to represent the entire mobile device communication network, regardless of the type of technology used in the communication network (e.g., GSM, Global System for Mobile communication, Code Division Multiple Access (CDMA), General Packet Radio Service (GPRS), Picture Communication Symbols (PCS), Code Division Multiple access (CDMA)), Universal Mobile Telecommunications System (UMTS), Long-Term Evolution etc. (LTE). The PLMN may control any base station with which it is in communication, and handle connections from cellular tower to cellular tower and from a cellular tower to a land-based phone system. While the term “cell” or “cellular” is used herein to refer to a certain type of mobile device communication protocols, this term is used in its broadest sense to include other communications systems such as personal communications service (PCS) protocol, and the Global System for Mobile Communications (“GSM” “CDMA”) protocol, and other similar communications protocols.

The OSAC system of the invention also includes one or more cellular phone switching cells and, thus, towers, as the phone is moved between geographic areas, allowing constant communication with the PLMN. Typically, a cellular phone, smart-phone, wearable communication device, satellite phone or any other type of handheld device has one or more codes associated with it, used to identify the specific phone, the phone's owner and the phone's service provider. However, when RFID readers become sufficiently miniaturized, they may be placed directly in the oral cavity. The oral RFID device, in this embodiment, becomes a smart oral device and removes the need for a smart RFID tag reader accessory or device through NFC as a part of the system, rendering it optional.

In a further embodiment, one or more databases, secure servers, or other devices is utilized to store and/or capture data. The database contains data collected from the OSAC system and any data input in the system including but not limited to data used to send alerts; here, data encompasses both data captured and collected from OSAC and data used to send out alerts. One or more software programs may collect, capture, and store data obtained from any of the OSAC interfaces. Some of this data will be publicly available and viewable and some of the data will be only for private viewing and available to the relevant users and healthcare providers. To prevent any loss of data, in certain embodiments, when connectivity is lost and data cannot be transmitted in real time to a central storage system, data is temporarily stored on the local device and transmitted to a central system after connectivity is restored. These software programs may be responsible for any and all data related aspects such as, but not limited to, data comparative with historical or previous data of same individual or other, management, characterization, filtering, transformation, sorting, processing, modeling, data mining, queries, browsing, inspecting, investigation, retrieval, integrating, dissemination, qualitative, quantitative, symmetric, asymmetric, normalizing, clustering, correlations, computer derived values and rages, simple or complex mathematical calculations and algorithms, analytics, statistical data, predictive data, integrations, interpretation, exploratory, finding abnormalities, performance, data products, consumer data, server data, visualizing and/or presentation in a variety of platforms. Here, data analysis also means software for that analysis for disease and other diagnosis and analysis for both humans and animals. Thus, this software may supplement or partially and/or fully replace a healthcare provider's input, such as that of a physician, veterinarian, etc., or in a non-medical context. It is understood by anyone familiar with the art that the present invention could lower the cost of health-care, and therefore, insurance companies could cover the cost of these devices for both humans and animals.

According to the invention, the OSAC system of tracking people and animals' biomedical condition is integrated with an analytical and/or predictive software component in order to determine or estimate, e.g., their possible points of origin, routes of travel, or proximity to specific events or locations. In a further aspect, all local, regional, domestic and/or international monitoring and notification systems are transmitted through the OSAC system.

Many clinical diagnoses are created based on the determination of blood biomarkers and blood chemistry levels. However, determinations of many biomarkers involve invasive procedures too destructive for certain patients already in poor health. Saliva and other oral biosensor sampling are relatively straightforward and the presence of various disease-signaling biomarkers in saliva has meant that it accurately reflects normal and disease states in animals and humans. However, current saliva technology presents some significant disadvantages which are greatly improved upon by this invention. In particular, saliva involves collection and sample determination, and in this invention, oral biosensors are cemented, implanted, or attached to parts of oral cavity/dental devices or embedded in oral devices. Some biological molecules are present at sub-detection amounts in either saliva or blood. At these levels, in certain embodiments, the biosensor captures these sub-detectable molecules and, when enough molecules are captured, an alert is sent so that the biosensor with captured bioactives or biologics may be removed for further analysis. The OSAC system of the present invention aggregates various wireless communications with the oral biosensors and biomarkers in order to monitor and notify users of one or more medical conditions. The OSAC system includes the integration of fitted oral devices not only equipped with various medical sensors (as previously described), but GPS/GPRS location positioning technology within the cavity of the mouth. In addition, the OSAC system includes the ability of the inserted oral sensor to communicate with other GPS/GPRS devices on the subject animal or human, or in close proximity to the subject animal or human.

In various embodiments, the OSAC system utilizes a multitude of RFID wireless uses of electromagnetic fields to transfer data for the purposes of automatically identifying and tracking tags attached to subjects. Orally installed biosensors of the invention communicate with RFID (NFC) reading devices outside the oral cavity. The tags contain electronically stored information. Some tags, in certain embodiments, are powered by electromagnetic induction from magnetic fields produced near the reader. Some types collect energy from the interrogating radio waves and act as a passive transponder. Yet other types have a local power source such as a battery, the battery, according to certain embodiments, being rechargeable while in the oral cavity, e.g., by motion, chewing, light, chemical reaction, oral biologics, or such, or by a combination of these methods. Thus, in these embodiments, they may operate at, e.g., hundreds of meters from the reader. Such RFID-NFC or positioning, micro- and nano-electronics communications, and other communication protocols, micro- and nano-devices, components may communicate with a plurality of reader/writer/emulator operational systems and platforms. A RFID is one method for Automatic Identification and Data Capture (AIDC) and may communicate with any network (satellite, WiFi, cellular, etc.) device depending on the specific biological requirements, needed bio-measures and/or budget considerations. RFID systems are sometimes classified by the type of tag and reader. A Passive Reader Active Tag (PRAT) system has a passive reader which only receives radio signals from active tags (battery operated, transmit only). An Active Reader Passive Tag (ARPT) system has an active reader, which transmits interrogator signals and also receives authentication replies from passive tags. In some embodiments of the invention, cellular, smart phone, or other wearable communication devices can function as a RFID reader. In the absence of WiFi, any other communication device can become a reader through a Bluetooth, smart phone, wearable communication device, communication or oral device, or other similar devices. In certain embodiments, can the OSAC system be activated or inactivated, by a voice response feature, or by touch commands or by automated biosensor-triggered commands sent after reaching a quantifiable threshold.

In additional embodiments, OSAC oral biosensors are inserted temporarily or permanently in a multitude of animals. Mobile alerts are provided based on metabolic data geared toward prevention, diagnosis, and treatment of conditions, diseases, and disorders of the oral cavity, the maxillo-facial region, and its associated structures as it relates to pets, farm animals, equine, land and maritime, wild, zoo animals (A-Z), etc., are provided. Additionally, the OSAC system of the invention includes systems and methods which interface and interact with combinations of “smart dental” and related devices. The invention incorporates fixed prostheses, crowns, bridges, braces and retainers, mouth-guards, horse-bits, child or adult pacifiers or pacifier-like devices, smart thermometers, a smart gauge, smart dipstick, smart rod, smart stick, smart device to collect blood or saliva, which can be not swallowed, with biosensors on the inside of the oral cavity and, RFID tags on the outside of the oral cavity, and/or animal toys, which can be not swallowed, with biosensors and RFID tag, or temporary/removable materials, used in dentistry and recreationally (e.g., tongue piercing, etc.). Biosensors and RFID tags can be attached, embedded, glued, inserted, etc. to a pre-existing oral device. The biosensors can be inserted by themselves into the oral tissue with or without a needle and/or plunger. This biosensor, when needed, can be removed by oneself at home using a piston, pulley, etc. or other devices. The dental device containing the biosensors can be disposable, clip-ons, stick-ons, adjustables, and/or removables. Teeth cleaning, mouth-wash, foods, or other ingested material could damage some sensors, so in certain embodiments the biosensors can be protected either by removal of the biosensor or oral device, or by covering if using airtight covers in the oral cavity itself. Using these covers and, e.g., suction devices, the sensors can be cleaned or rejuvenated in the oral cavity without need for removal. In individuals without teeth, such as babies, the device can be attached to, e.g., the gums. Patients can install, remove, and care for the sensors themselves by use of moldable plastics or other moldable materials, adhesive strips, etc. The OSAC alerting, monitoring, notification and reporting systems and other functions are configured to communicate through all application programming interfaces, e.g., cloud networks (APIs).

The following are examples of various OSAC system communication mode classifications to which the invention is applicable:

1. Real-Time or Near-Time Slumber to Alerts Mode.

In this mode, diagnostic data is collected in real time, and real-time alerts are sent based on deviations from set and/or preset values and/or ranges determined by a healthcare provider. These values may be unique to each situation and to a particular patient, both human and animal, for each oral biosensor application. Further, local, regional, domestic, or international monitoring and notification may be transmitted through the OSAC system. Listed below are examples of embodiments of this invention; the ranges and preset values used here are only uses as examples. It is understood by anyone familiar with the art that many more examples are possible with both humans and animals without departing from the scope of the invention.

In one such embodiment of the present invention, a metabolic range is set or preset on a wireless device exemplified by the following scenarios: OSAC presets a biosensor device in the mouth of a dog named “Bogie,” who has had several health problems and survived harsh environmental conditions since birth. The OSAC biosensor is “preset” for a temperature of 101 to 102.5 degrees Fahrenheit (38.3 to 39.2 degrees Celsius). The device is inserted into the mouth of the puppy. The oral device is also preset to include Bogie's optimal heartbeats per minute. Additionally, the OSAC device is preset to include his breed and age. In young puppies, the heart rate is about 220 beats per minute. It is determined by the veterinarian that Bogie's preset should range from 218-225 beats per minute. In this OSAC scenario, the “preset(s)” are described as any and all biomedical health monitoring through a mouth and/or oral cavity. Oral cavities are linked to ear, nose, throat, and neck diagnoses, and related presets may be added. In this embodiment of the present invention, the inserted OSAC device is in sleep mode and is activated if or when the heartbeat, for example, hits a threshold below 218 beats per minute or above 225 beats per minute. If the range is either above or below the preset value, one or more alerts is activated and transmitted through the wireless system. The alert may also be displayed on one or more devices. For example, taking the same scenario, Bogie's owner may receive the mobile alert as well as the veterinarian in real-time or near-time to the event. In accordance with the invention, any animal or human and any medical or biological situation may be addressed in the aforementioned manner. When presetting values or ranges, in addition to the type of species, other factors may be included, such as the age, environmental location, and other relevant factors germane the animal may also be considered. For example, adult dogs at rest maintain a heart rate of 60 to 160 beats per minute, rather than 218 to 225 beats per minute typical in a puppy, so different heart rate values for an adult dog and a puppy may be preset. In addition, the OSAC system of the invention may interpret various factors such as location, environment, stress levels, dehydration, infections, toxins, pathogens, allergens, and numerous other factors in establishing such presets.

In this example of an embodiment of this invention, abuse or disease in animals, children, the elderly, and the disabled is detected. A pacifier is, in this embodiment of the invention, used to detect disease or abuse. A child in a day-care center develops an infection and her body temperature becomes elevated. The system's temperature sensor transmits a bi-directional alert to the parents and/or other authorized people. A tracking sensor is alerted, for example, if a babysitter takes the child away from home or if the parents want to monitor the baby's sleep. The baby's feeding patterns while the parents are away from their child can be monitored in a similar fashion. According to this aspect of the invention, a variety of biosensors may be used to detect abuse and neglect; dehydration, electrolyte imbalances, reduced nutrient levels, decreased blood glucose, altered heart rate and/or blood pressure are some examples of symptoms that may be monitored for signals of abuse and neglect; OSAC in real-time or near-time with one or more of these biosensors may be used to send alerts to healthcare providers or relevant law enforcement personnel.

Most critically, endangered animals (e.g., see Table II) are subjects of poaching which results in the significant decrease in numbers of these animals, thus, endangering the species survival. In this embodiment of the instant OSAC invention, OSAC may be used to rescue these animals in real-time or near-time. Often, a drastic and sudden change in an animal's body temperature, heart rate, blood pressure, electrolyte imbalance, dehydration, blood or salivary chemistry, and/or blood loss suggests that the animal is in stress due to injury or danger, and therefore, may signal that a poacher is victimizing the animal. Deviations in the preset range for any of the above aforementioned symptoms may result in the sending of real-time or near-time OSAC alerts to authorities and security personnel. The authorities and security personnel may intervene; and potentially, according to the invention, drones and/or security teams may be deployed to assess the situation and rescue the animal.

Chronically or otherwise sick people who need constant supervision by a healthcare provider also may benefit from this invention. For example, diabetes is a chronic illness where blood glucose levels need to be regularly monitored and controlled. When elevated blood glucose levels are observed, insulin should generally be administered to a diabetic patient to reduce and regulate the blood glucose levels. In this embodiment of our invention, insulin pumps are deployed to interface with OSAC so that glucose oral sensors may constantly monitor blood glucose levels. Also, normal fasting glucose levels in adult humans are generally known to be between 70 and 100 mg/dL and the non-fasting value level is about 140 mg/dL. For example, when glucose levels are above preset value of 140 mg/dL, according to the invention an alert is sent to an insulin pump which administers a preset amount of insulin to the patient and alerts a healthcare provider. In one embodiment, the amount of insulin administered is changed remotely as needed by the healthcare provider. However, if the blood glucose levels are below 70 mg/dL, an alert may be sent to the patient, caretaker and/or healthcare provider, recommending the patient to ingest some simple sugar containing foods.

In the above-mentioned embodiment of the invention, the glucose dispensing pump may be substituted with another drug dispensing pump in certain situations. For example, several drugs may cause severe side effects, but are also life-saving. Several cancer treatment drugs, e.g., may cause leukopenia, a condition in which white blood cells (WBC) are dramatically reduced. WBC has a normal range of 4.5K to 10K per microliter. Because WBCs are important in the prevention of infections, leukopenia may cause severe infections and even death. In another embodiment, cancer drug dispensers may be set to interface with WBC-oral biosensors, and when WBC levels fall below a preset value, e.g., significantly lower than 4.5K per microliter, the dispenser pump stops, or reduces the amount of the cancer drug dispensed until the WBC levels reach 4.5K per microliter. However, if the drug is not administered by a pump, but is dispensed as an oral pill, alerts may be sent to healthcare providers and the patient recommending them to reduce the drug dosage, or to completely stop until the WBC levels reach a normal range. In this embodiment, the OSAC system of the invention may communicate with both a drug dispenser pump and nano-particles in the medication to determine an exact and accurate blood drug concentration.

The OSAC system of the invention is useful in other more complex and life-threatening situations requiring more than a single biosensor. For example, in treatment of congestive heart failure, cardiac arrhythmia, and post-cardiac surgery heart-attack prevention, digitalis drugs are used. The active compounds in digitalis medications are the cardiac-glycosides. Cardiac-glycosides are the most studied positive ionotropic drugs that increase the force of heart muscle contraction; strong heart contractions lead to increased cardiac output and better heart function. However, this drug has a very narrow therapeutic window, so slight increases in blood glycoside levels may cause the drug toxicity to manifest itself in severe and irreversible side effects, which may even result in death. Additionally, glycosides are very sensitive to blood potassium levels, and low potassium levels enhance the drug's side effects. In this embodiment of the OSAC system of the invention, real-time or near-time OSAC with biosensor for glycoside and potassium is placed in the patient's mouth and alerts are sent to a healthcare provider and/or patient if either one or both blood glycoside and/or potassium levels reach near toxicity levels. The alerts may lead to a reduction in digitalis and or potassium doses. This example of an embodiment of the OSAC system of the invention may shorten the hospitalization of post-surgery patients who are on digitalis, and thus, significantly reduce the cost of their care. In this embodiment of the inventions, any two or more drug interactions may be determined and the OSAC system may communicate with different nano-particles in the drugs.

Another embodiment of the OSAC system of the invention uses several biosensors and other devices located on or near the animal or human. Cisplatin is known to be used in treatment of lung, testicular, bladder, and ovarian cancer. Unfortunately, this drug causes severe side effects, including hypertension, ischemia, and atrial fibrillation. This situation is a complex one because the drug may cause several life-threatening side effects that need to be monitored simultaneously with the patient's blood cisplatin concentrations. In this embodiment, an OSAC system is installed with biosensors for cisplatin concentration detection. The drug can also contain nano-particles which, when present in the oral cavity, stomach, or circulatory system can be in communication with an OSAC system. Cisplatin concentration detecting biosensors can be coupled with one or more of the following biosensors: blood pressure for hypertension, oximeter for ischemia, and heart rate for fibrillation. A preferred diagnostic tool for atrial fibrillation is a cardiac monitor hooked to the chest of a patient which constantly or frequently monitors heart function. Therefore, in this embodiment, these patients are optionally hooked up to a cardiac monitor. The OSAC system described herein sends alerts to a healthcare provider and/or to a drug dispensing pump whenever the patient suffers from one or more side effects for which intervention is indicated. The alerts may optionally alter drug dosage to alleviate the side effects. Additionally, the OSAC system of the invention optionally contains other drug dispensing pumps or devices that administer additional medications to alleviate side effects. For example, pumps to dispense antihypertensive or anti-arrhythmia medications are a potential part of the OSAC system described herein. A defibrillator device is also optionally a part of the OSAC system to treat and eliminate cardiac fibrillations. Because infections may result from treatment with cisplatin, alerts about elevated core body temperature can be bi-directionally transmitted to a physician who can further check the patient for possible infections. This example exemplifies how the OSAC system may use multiple routes to diagnose one or more problems and treat several symptoms simultaneously and in real- or near-time. In this embodiment, it is possible that the OSAC system of the invention communicates with the drug dispensing pump, several medical devices, and nano-particles in the medication to get a very accurate overall health picture of a patient, and this overall picture is important to save life.

Severely sick people often suffer from more than one illness. Therefore, they are frequently administered several medications that interact with each other changing their effects and causing several additional side effects. For example, a person suffering from cardiovascular disease with high cholesterol levels while also being treated for a cancer, may be receiving statin drugs to reduce cholesterol levels, which may render cancer chemotherapy being received more potent by increasing the blood concentration of the chemotherapy drug, thereby enhancing the potency and toxicity of the chemotherapy agent. In this embodiment, the OSAC system of the invention utilizes biosensors for the following: statin drug or nano-particles in statin concentration detection, blood lipid profile or cholesterol concentration detection, and the chemotherapy drug such as cisplatin or nano-particles in cisplatin concentration detection, could help determine the exact amount of chemotherapy drugs and statin drugs being administered to treat both diseases simultaneously.

Animal research forms an integral part of basic and pharmaceutical research. These researchers may use the OSAC system herein to collect more data from the research animals, and therefore, reduce the number of animals needed for use in their research. In one embodiment of the OSAC system as described, a scientist discovers a new pathway to treat diabetes by inhibiting a function of a protein X, and he wants to test a number of X inhibitory compounds. In cell models, compounds #1205, 1252, 0501, and 0512 were shown to be good inhibitors in descending order, decreasing strength with 1205 being the strongest and 0512 being the weakest inhibitor. Thus, he now wants to test these compounds for their bioavailability and toxicity in animals and decide which compound is the best candidate for the treatment of diabetes in humans. Unaware that compound #1205 could cause a transitory increase in heart rate as initial bioavailability was very high, and this transitory elevated heart rate could result in a high incidence of heart attacks and fatalities in humans, he checks heart rate intermittently and not constantly after drug administration in mice. However, the increased heart rate had already returned to normal before the first heart rate measurements, so he has missed a very severe and life-threatening side effect. In fact, unknown to him, compound #0501 is the best candidate because it had the least side effect profile. After administering the drug for a week, he scarified the animals to investigate effects of the drug on various organs, but the heart tissue in these animals had already recovered. Therefore, these experiments were wasteful, and the scientist did not realize that the animals had suffered. In this example, the drug could not get FDA approval even though the new pathway was very encouraging. However, if real-time OSAC according to the invention used with a heart rate biosensor had been available to the scientist, he would not have missed the window of severe side effects and would have chosen compound #0512 for further drug development. In this scenario, this compound could have led to the development of a new diabetes drug which potentially could have been very helpful many humans and other animals suffering from diabetes, but was tragically missed. This embodiment of the invention is designed to help avoid such current gaps in research and development, as well as mitigate unnecessary overuse of test animals.

In yet another embodiment of this invention, nano-particles are used to diagnose cardiac function and blood flow related diseases such as, but not limited to, obstructions in blood vessels caused by plaques which are precursors of atherosclerosis and stroke. Nano-particles such as dyes, fluorophores, etc. can be injected, in the arm, for example, and the movement of the nano-particles through the vessels and heart can be followed and communicated through the OSAC system. This can be done in real life when the person is doing daily activities, and not be limited to a hospital setting with imaging equipment. Following daily life for longer periods is more useful in several medical conditions. Various blood vessel and cardiac functions can be calculated by complex mathematical equations known to, or which can be developed by, one skilled in this art. A plurality of sensors, biosensors and RFID or other micro- and nano-communication and energy harvesting, micro- and nano-energy storage components may be installed in any dental device and/or placed in any oral cavity, depending upon the medical or diagnostic intent. In addition, in other embodiments, the OSAC systems of the invention may be used in conjunction with other smart wearables, attachables and insertables anywhere on the body of the same or another individual to form a network of smart wearables or other medical devices used by a collective group such as a basketball or other sports team. A single or a set of wearable or other medical devices can be placed on the team member/s that send alerts and communications, through the OSAC system in real-time or near-time, to the coach and/or the team's healthcare provider, to ascertain the medical and/or diagnostic condition of each team member for the best sports outcome. The data collected may be used to determine the health status of each individual and/or to compare with another team member or collectively as a team. For example, a player might be very important to the successful outcome of the game but he is injured; based on his bodily functions as determined by the OSAC system, his performance can be predicted and the information used to help the coach to make strategic decisions. In another example, the coach compares statistics between two or more players on the team to make a decision about their performance.

Still, in yet another example of this embodiment, the coach compares the collective statistics of the team as a whole under varying conditions such as weather, geographic location, team acclimation, etc. The OSAC system of the invention, in this scenario, in certain embodiments, also communicates with environmental sensors on inanimate objects to determine performance characteristics of the athletes. In this scenario, sensors for core body temperature, for stress and fatigue related inflammation, cortisol, for stress, muscle tone for muscle function, electromyography, EMG, for muscle function, stamina, performance, lactose for fatigue, inertia for gait, performance, accelerometer and gyroscope for gait, speed, performance, oximeter for fatigue, heart rhythm for heart function, heart rate or pulsometer for heart function, and respiration rates for fatigue, stamina, and performance, are all used contemporaneously. The OSAC system also provides artificial intelligence models which can augment a cognitive map which permits an individual to acquire, code, store, recall, and decode information regarding the relative locations and attributes of phenomena in their spatial environment for one or more players. Measurable data may also be generated on groups or sub-groups of an opposing team. The OSAC system can provide for adjustment of an individual's performance based on, e.g., wearable data analysis. The coach may correct bad habits of his players through the OSAC system. When a player shoots from the free throw line in basketball and misses, for example, this can be corrected when in practice by the coach through the player's wearable network as previously described or through an ear piece worn by the player.

The OSAC system can make available the ability to integrate data concerning numerous team and individual sports models as exemplified by the following definitions:

Using a sports example of OSAC, a baseball player could be in a batting slump due to habits developed over time with his batting swing or in not detecting the baseball as thrown. The OSAC system could, for example, detect the smart baseball, with the velocity, and track (curve, twist, etc.) and direction of the baseball. The OSAC system could signal the batter to swing the bat in a certain motion, change and alter his or her stance, signal him or her when to swing the bat, calculate not only the projected speed, but delivery to the batter's box. In addition, the OSAC system could signal through vibration, Automated Motion Directives (AMD) to direct the batter to optimize his timing to increase batting average. It is obvious to anyone familiar with the art that, given these basic principles developed by applicants, hundreds of additional examples could be used to exemplify the present invention. In addition, as previously described, given the same example, the batting couch could in real-time or near time use OSAC analytics to help the batter to modify his or her swing in order to enhance performance.

Individual wearable/attachable data used as a team component from a plurality wearable “inter” and “intra” devices, External Structures (ES) and Smart Sports Equipment (SSE). Smart Inter Devices (SIRD) for the purpose of this definition is devices which can be embedded in the mouth cavity, for example. Smart External Wearable Devices (SEWD) are defined as devices which can be intertropically (two or more convergent wearables for cross-functionality and functional prioritization) worn on the body or near the body. External Structures (ES) can be defined by any structure such as, but not limited to, a playing field, stadium, racetrack, court, including any indoor or outdoor environment which facilitates an athletic or organizational team. Smart Sport Equipment “SSE” is defined as any equipment needed to facilitate their respective sport and athlete; such as smart-balls, smart-hoops, smart-base-boards, etc., and any other device which facilitates their respective sport. It is understood by anyone familiar with the art that all sports equipment smart-balls can be tracked, their movements traced, and integrated.

The OSAC system, in certain embodiments, integrates SSE, SIRD, SEWD and environmental and external facility (ES) data though data captures received from each in order to seamlessly and accurately draw information from each component.

A basketball game training session could help exemplify the OSAC system as it relates to sports exemplified by the following:

San Antonio Spurs players are outfitted wearing smart trackable wearable SIRD—biosensor equipped mouth-guards, as well as SWED—smart arm braces, smart socks and the clothing etc. When practicing, a guard brings a smart-ball (SSE) containing sensors down the court, passes the ball to a forward, while an opposing player misses his chance to block the pass. In one embodiment, the player's wearables vibrate on the right hand wearable support to signal the player to shift to the right. Thus, in this embodiment, the OSAC system provides the player with a form of direction or movement (height, right, left, front, back etc.). In addition, the OSAC system can alert for spatial proximity with other players, provide for motion correction when passing the ball or taking a shot at a smart-hoop (SSE), and provide for other inputs sought by the coach, for example. If a player misses a shot, the network of OSAC wearables on the player's body can help correct the movement by vibration or other methods. Other methods can integrate computer generated images of each player with actual inputs from SWED, SIRD and SSE with high-definition video and images, shape context and flow models based on each player, or the team as a whole. Furthermore, the ES environment can be tracked by pixel location including the YY and XYZ coordinate for each player, or the team as a whole. If a player is not performing satisfactorily, the network of OSAC wearables on the player's body can help correct the movement by changing behaviors patterns and performance mechanic to optimize players' performance by providing greater depth and enriched of information to coaches, managers, staff, etc.

Team data may be derived by creating a composite out of wearable devices data based on individual player performance. The OSAC system can also provide one or more biometrics in order to determine a player's O₂, hydration, pulse, and/or temperature, individually or as a collective. For example, the player may insert an oral monitor containing biosensors which, through the oral cavity can determine a player's energy level and when he needs to be replaced. The OSAC system can also account for external factors such as, but not limited to, altitude, the opposing teams courts and fans, noise levels and other external factors.

Measurable data on groups or sub-groups of one team. The OSAC system, in certain embodiments, also provides the coach or manager a composite map of how each player's performance can be enhanced and optimized by resting him at certain times through use of the oral biosensor insert. Thus, sports, motions, biosensor data and visual components derived from multiple inputs are integratable through the OSAC system as previously described.

Another example of the OSAC system is exemplified by a basketball player named James. James Jersey, No. 1, is wearing smart devices when training for his basketball team. His teammates are also wearing smart OSAC devices and the squad has been split in half for a practice scrimmage. The OSAC system measures bio-parameters for all 10 players on the court. The OSAC database is concurrently running in real-time or near-time analyzing movement efficiency for both individual and team performance. The OSAC system also compares each player's current performance with his historical database, historical performance, biomarkers and bio-parameters.

Core body temperature, pulsometer, oximeter, cortisol, respiration rate, accelerometer, gyroscope, blood sugar or glucose, and lactose biosensors could be installed inside the oral cavity. Additionally, muscle tone, EMG, lactose, inertia, gyroscope, and accelerometer sensors can be installed at multiple locations on both legs and leg muscles. To get very accurate readings, respiration, heart rate and rhythm biosensors could also be placed on the player's chest and, perhaps, some sensors could be placed on inanimate objects in the field such as on a smart ball, hoop, clothes, etc. Variations of this embodiment can also be used for race horses where oral biosensors may be attached to a horse-mouth-bit and a RFID reader can be installed on the horse-rein.

The court itself may be panelized with useful sensors and the OSAC system could provide the coach with real-time or near-time information to reposition players to help their defense. In addition, the OSAC system can help train the guard by giving directional inputs such as, but not limited to, vibrations to directionalize a player's movement.

2. Manual Control Diagnosis Mode.

In addition to the above, the OSAC system user, caretaker, healthcare provider, and others are able to manually activate one or more biosensor data-points in order to visualize and analyze information inputs from the host animal or human. Local, regional, domestic, or international monitoring and notification is optionally transmitted through the OSAC system of the invention. Ranges and preset values will be determined by the user, caretaker, healthcare provider, or other, and the ranges and preset values discussed here are only used as examples, apply to additional information inputs, and apply to both humans and animals.

### Example #1

Pet owner turns on the OSAC alert system when leaving her pet (Bogie) home alone. In case of any stress to the animal, the owner receives an alert about changes in the pet's health and disease status, such as any alteration in its temperature, heart rate, blood pressure, dehydration, and oxygen level. Bogie's owner has an emergency and has to leave him home alone. She forgets to put food and water out for Bogie but manually turns on the alert system before she leaves the house (or remotely does so after leaving the house). Dehydration and decreased heart rate are symptoms of missed meals and lack of water consumption. The OSAC system of the invention sends alerts about the decreased heart rate and dehydration of Bogie. Bogie's normal respiration rate at resting is generally between about 10 to 30 breaths per minute with an average of about 24 breaths per minute. However, puppies and small dogs have a normal respiration rate of about 15 to 40 breaths per minute, and when panting, can be as high as 200 breaths per minute. When Bogie's respiratory rate is below 10 breaths per minute, an alert is send to the owner and his owner knows to call a neighbor to feed Bogie and fill his water bowl or other such suggested or pre-determined alert response.

In one embodiment of this invention, a baby, Peter for example who is a 4 month old baby, could be exposed to other children suffering from lung bacterial or another infection; hence, the parents want to know if the baby contacted the infection so they can take him to their pediatrician for check-up and timely start a course of antibiotics or other medication to keep the infection and symptoms at a minimum. The parents could manually turn the system on when needed.

### Example #2

An OSAC alcohol monitoring system is provided for humans. Paul is going out with his friends and is not sure if he could drink alcohol, so he activates his OSAC system. When he drinks alcohol, the alcohol in his mouth causes the alert system to begin sending communication to another secondary biosensor, located either in the oral cavity or any other place on his body, to begin monitoring his blood alcohol levels at predetermined desired levels. Therefore, the OSAC system of the invention using preset values of blood alcohol concentrations (BAC) will send out alerts to Paul's pre-designated people. In this case, his wife could receive the alerts and pick him up if needed. It is understood by anyone familiar with the art that BAC between 0.01% and 0.05% is considered hazardous for driving, so if his BAC levels are below 0.01%, his wife would receive a second alert and she would not need to pick him up. However, if his BAC levels are above 0.05%, she would get a second alert and she would be given a higher-level alert to pick him up. Such an OSAC system is an option for law enforcement use in addressing chronic alcohol-related offenses.

### Example #3

John suffers from high blood pressure and uses the OSAC system described to remind him to practice relaxation techniques to avoid hazardous symptoms of elevated blood pressure. In this example, John suffers from cardiovascular diseases including high blood pressure and his blood pressure is controlled by medications. However, his job has added stress to his life, with Mondays and Wednesdays being the most stressful, possibly causing his blood pressure to become erratic. His doctor recommends deep breathing exercises and meditation for short periods of time during stressful moments to relax and reduce his blood pressure. Elevated blood pressure due to stress is known to precipitate heart disease and even cause stroke, so John's deep breathing and meditation assist him to relax during high stress moments. He, the described, activates OSAC system, using blood pressure and heart rate biosensors, on Mondays and Wednesdays, or at other times of likely high stress, to send alerts to him when his blood pressure reaches values set by his healthcare provider (e.g., 150/90 mm of Hg for him). When he receives alerts from the described OSAC system, he takes a few minutes to breathe deeply and meditate, thus reducing the possibility of elevated blood pressure and its consequences.

### Example #4

A trucking or airline company wants to ensure that drunk drivers or pilots do not drive their vehicles. Therefore, at the start of their shift, the driver or pilot starts the OSAC system and stops it at end of the shift, and if he ingests alcohol, the OSAC system will send alerts to the company and appropriate action is taken. When the described OSAC system is started, it also begins measuring OSAC levels frequently at intervals preset by the company and sends alerts to the company.

### Example #5

Nina wants to lose weight. However, due to her traveling job, she finds it difficult to regulate her food and caloric intake. When she is traveling, she activates her OSAC system of the invention with biosensors for blood sugars, total blood lipids, and blood proteins and/or amino acid levels. Using the values obtained from these biosensors and making appropriate mathematical calculations known to those skilled in the art, the OSAC system computes the amount of calories she consumes at every meal. The described OSAC system sends Nina reports after each meal with a breakdown on her calorie and dietary consumption. When she has consumed the required intake of calories and/or other nutrients, she is alerted to stop ingesting additional food and drink, or to exercise to burn off extra calories. In this embodiment of the invention, the OSAC system may communicate with other smart devices and apps such as Fitbit, etc. Weight gain and obesity result from imbalances in food and caloric intake and caloric output, e.g., by exercise, yet most programs, such as Fitbit, only address caloric output. Biosensors used according to an embodiment of the invention, can help establish healthy eating habits by evaluating food in-take habits, and chewing and analysis of ingested food. Biosensors for movement and inertia, accelerometer, gyroscope, pressure, oral digestive enzymes, etc. can determine mechanisms of chewing through complex mathematical equations. Food components can also be determined by analysis of ingested foods by including biosensors for carbohydrates, proteins, and fats. This analysis helps in determining poor eating habits and establishing reasons for such weight gain. Optionally, in one embodiment, taste buds are modulated biosensors which assist in establishing food strictly according to methods known in this art, thus the method helps in developing and successfully operating such a weight loss program. This part of this embodiment can also be alerted by other modes such as real or near time mode.

3. Programmed Automated Diagnosis Mode.

In addition to the aforementioned modes, the user of the described OSAC system can automatically activate one or more biosensor data-points in order to visualize and analyze information inputs from a host animal or human. In this embodiment, the OSAC system is set to any time schedule (second, minute, hour, day, week, month, etc.) in order to activate or monitor the medical health of any species or patient. Local, regional, domestic, or international monitoring and notification may be transmitted through the described OSAC system. Additional examples of embodiments of this invention are possible with both humans and animals and the ranges and preset values discussed here are only used as an example. The example used herein may be utilized by both human and animal patients.

Additionally, general symptoms, such as body temperature, and/or heart rate, optionally may be monitored to predict early onset of various chronic diseases, infections, or emerging diseases. In one embodiment of the invention, core body temperature levels can routinely be monitored. Monitoring during any specific time of the day may be programmed. Basal normal core body temperature is about 99.4°, and when core body temperature is above this, an alert is bi-directionally transmitted to the healthcare provider and other authorized personal. This embodiment can also be modified, e.g., to determine ovulation and fertility time periods for a woman. Specific symptoms, such as elevated glucose and hemoglobin A1C, that predict diabetes may be monitored regularly. In one embodiment of the invention, blood glucose levels are routinely monitored. Because fasting blood glucose levels are the best predictor of diabetes, daily monitoring during early morning before food ingestion may be programmed. Normal glucose levels between 70 and 100 mg/dL, and levels above 100 mg/dL send an alert to the healthcare provider. Hemoglobin A1C levels in the body reflect an increase in blood sugar for two to three months, and thus, these should be measured approximately once every one or two months. Hemoglobin A1C levels above 4% to 5.9% may be set to cause the described OSAC system to send an alert to the healthcare provider.

In another embodiment of this invention, babies at a high risk for infections can be monitored for an elevated temperature, one of the first symptoms of infection. Cystic fibrosis, e.g., increases the risk of infections and babies with this disease may be monitored daily for elevated body temperature through the smart pacifier. When the baby's body temperature is elevated, she may be further tested and, if appropriate, treated immediately. For such babies, this could be a life-saving device.

In a further embodiment, the specific measurements of a symptom that helps predict any of a variety of diseases is set on alert. For example, elevated levels of C-reactive protein are an inflammation predictor. C-reactive protein could be a symptom for variety of diseases such as metabolic diseases, infections, etc. In one embodiment of the invention, blood C-reactive protein levels are routinely monitored, and when the levels are above a preset value, alerts are sent to the healthcare provider to further investigate the cause of this increase.

Endangered animals could benefit from this invention. As an example of a possible embodiment of this invention, the described OSAC system is deployed in saving several thousand gorillas from death by Ebola infection. The Ebola virus spreads more rapidly among apes than humans, and the earliest symptom of the infection is an elevated body temperature. Sick gorillas that are infected with the Ebola virus and have increased body temperatures may be isolated when the deployed OSAC system of the invention sends out alerts about their elevated body temperatures. The isolated gorillas that died from the Ebola infections are then disposed of properly in order to reduce further spread of infection among other animals. Hence, OSAC frequent temperature monitoring according to the invention may be used to assist in identifying potentially infected gorillas that can be further tested and isolated to prevent the disease from spreading and more animals from dying.

In yet another embodiment of this invention, the described OSAC system is used to help the sick who are prone to fluctuation in other symptoms such as electrolyte imbalances. For example, alterations in blood concentrations of sodium or potassium are predicting a variety of diseases such as kidney diseases, malnutrition, dehydration, and loss of fluids by either vomiting and/or diarrhea. Monitoring medications for appropriate alteration in their concentrations are also possible for healthcare providers who want to regularly monitor their levels for some patients using the described OSAC system. When such imbalances are alerted, the healthcare provider is able to intervene to correct the imbalance.

All animals, elderly and chronically ill people, human children, disabled humans, patients in hospice, and mentally challenged people who are in pain are able to benefit from the described OSAC system. In one example of an embodiment of this invention, biosensors for pain and symptoms of pain, such as heart rate, blood pressure or other biomarkers are made a part of the OSAC system, and a pain medication dispensing-pump interfaces with the OSAC system of the invention. An alert sent to the healthcare provider, caretaker, and/or others when the pain is elevated based on biosensors. An alert is also optionally sent to a dispensing-pump to dispense appropriate medications. Alerts will be sent to the healthcare provider about elevated pain and pain medication dispensing who then ensure the safety of an administered drug dose and adjust the dispensed dose as needed. Pain is one of the most agonizing experiences for all beings and eliminating it is, perhaps, the most humane for all beings suffering from it. This benefit also reduces costs associated with monitoring of prescribed narcotic pain management. Moreover, over use and inappropriately higher narcotic and pain medication dosage is commonly abused, so appropriate narcotic and pain medication dosing and use is a significant medical issue. Thus, the describe OSAC system, with it automatic dispensing components, makes available an enhanced medication compliance benefit. In this embodiment, the described OSAC system optionally communicates with nano-particles in medications such as addictive drugs, pain-killer, narcotics, etc.

In another embodiment of this invention, pressure-detecting biosensors are placed at different parts of teeth to measure grinding and teeth damage associated with it and alerts sent through the OSAC system whenever pressure increases to the level of damage. Based on findings from the OSAC system, dental guards, fillings, etc. are optionally placed or constructed to prevent damage to the teeth, gums, oral cavity, etc. This is particularly useful for those most at risk for dental injuries, such as athletes, or high stress workers prone to grinding.

In another embodiment of this invention, several respiratory diseases such as sleep apnea, snoring, etc. are diagnosed and treated. Biosensors to measure respiratory rate and muscle tone or weakness during sleep can determine if a person has sleep apnea because respiration stops during sleeping in sleep apnea. The OSAC system detects this stopped respiration and sends an automated signal to respiratory muscles to stimulate them and correct the sleep apnea. The diagnostic methods used today are very uncomfortable and cumbersome.

4. Geographic Analysis Mode.

In addition to the above, in a further embodiment, a geographical OSAC dissemination and locator mode is also utilized in order to, for example, monitor and analyze a herd of cattle, a herd of sheep, or other animals on a protected geographical range. Local, regional, domestic, or international monitoring and notification is optionally transmitted through the described OSAC system. Many examples of embodiments of this aspect of the invention are possible with both humans and animals and the ranges and preset values discussed here are used only as examples. The example used here may be used by both human and animal patients. Training and performance of athletes and race horses is impacted by altitude, racing conditions, the jockey and training etc. OSAC oral biosensors as described herein can be designed to outfit humans as well as animals. Taking the horseracing example, the horse's mouth bit which has existed for hundred years with little change can be made of material which serves the same purpose, guiding the horse while, e.g., reading the animal's medical diagnostics. Based on medical diagnostics such as, but not limited to, O₂ levels, temperature, hydration, and heartbeat a protocol is established which greatly assists the horse's training and performance. According to the invention, the race horse's diagnostics and performance data, e.g., can be read by the trainer and a historical comparison with past performances made. In addition, a horse's variable speed when training or while racing can be compared to sensor data which allows its trainer to alter the animal's training routine, diet, and race strategy, for instance. Thus, the OSAC system mode of the invention is important to help determine proper precautions which enhance the optimum performance under various geographical, medical, and performance conditions.

In this embodiment, isolation of infectious diseases to a certain region is made possible which prevent diseases from further spreading. For example, when Ebola began spreading from the African nation of Liberia, people leaving the country were monitored for fever, by questionnaire since fever is an early symptom of the Ebola infection. However, if the described OSAC system using a temperature biosensor is used in people living in the infected area, or who were traveling to other countries from Liberia, general panic may be prevented or mitigated and even lives saved. Moreover, instead of self-monitoring, the OSAC system is useful for people who are in contact with infected patients and first responders. The OSAC system using a temperature biosensor used within Liberia, or a similar epidemic, could have speeded containment of the Ebola infections or similar epidemics.

Certain diseases can be linked to a local diet, environment, cultural factors, disasters, etc., and the described OSAC system may be applied in these situations. For example, the nuclear spill, such as the one due to an earthquake, in the Kashiwazaki-Kariwa Nuclear Power Plant in Japan, caused humans and animals to be exposed to high levels of radiation. Exposure to high levels of radiation causes a variety of cancers. Early detection of cancer enhances the prospects for a good outcome of most cancer treatment. In one embodiment of this invention, a panel of various cancer biosensors is placed in the oral cavity of the exposed people, and the OSAC system alerts healthcare providers about the development of new signs of cancer in these individuals.

In order to increase the number of critically endangered animals, females that become of child-bearing age must be kept in good health. In one embodiment of this invention, these females of child-bearing age are connected to the OSAC system with biosensors for testing positive pregnancy. For example, Rhinos in Sumatra are endangered, so all female Rhinos of child-bearing age may be connected to an OSAC system with biosensors for testing positive pregnancy. When alerts of pregnancy are received, the authorities take measure to enhance or ensure the good health of the mother and that healthy baby Rhinos are born by providing the mother with appropriate nutrients and medication in the wild. This could increase the Sumatran Rhino population.

5. Species Classification Analysis Mode.

In addition to the aforementioned example, in a further example, a specific endangered species is monitored through the described OSAC system network. Animals at the verge of extinction would benefit from this invention. These animals are often tagged with sensors on their skin, such as their ears or soles of feet, and monitored. Tagging these animals in their oral cavities offers additional protection for the sensor from unfavorable external elements. In addition to monitoring the animal's survival, the biosensors also are capable of monitoring them for their disease, nutrition and overall health status. Therefore, appropriate intervention becomes available to increase their life-span and reproduction. Overall, these biosensors reduce the risk of extinction for several animals. Many different embodiments of this invention are possible with both humans and animals. Local, regional, domestic, or international monitoring and notification may be transmitted through the described OSAC system.

This mode of the invention is specifically important because not all animals have the potential of developing the same diseases. Dogs, for example, could become ill from ingesting onions and chocolate, which in turn are very beneficial for humans. Cats, unlike dogs, but like humans, develop metabolic diseases easily and at a younger age, such as diabetes, cardiovascular problems, and other obesity-related diseases. Pathogens for tooth and gum infections are different in pet animals than in humans. Several pathogens are species specific, meaning that they cause disease in one species but not in others. For example, Coronavirus (SARS-CoV) cause severe acute respiratory syndrome (SAR) in humans, but not in other animals such as domesticated cats.

6. Specific Disease or Situational Alerting Mode.

In addition to the aforementioned modes, the OSAC system of the invention is a source of disease specific or situation specific modes of operation. Local, regional, domestic, or international monitoring and notification are transmitted through the described OSAC system. Among the diseases, e.g., may be hoof and mouth disease in one or more animals and geographical areas. Additional examples of embodiments of this invention are possible with a variety of diseases and situations in both humans and animals. Examples of this OSAC system mode include: heart rate.

### Example #6

Farm animals are monitored for diseases and exposure to pathogens, especially those that could be passed on to humans, such as example mad cow disease. Animals that are exposed to mad cow disease (or other) infectious diseases are isolated and treated before the disease is transmitted and spread to others in the herd. The diseased animals are prevented from entering the human food chain. In this example, farm animals are routinely monitored for symptoms for early disease onset. Elevated body temperature is an early symptom of most infectious diseases. Whereas, an altered heart rate is a symptom of several diseases, including, but not limited to, metabolic diseases. It is known that infections are the most common disease in farm animals. These infectious pathogens are hazardous to both animals and humans if they enter the food chain. In this aspect, farm animals are programmed for daily monitoring of their temperature and/or heart rate. If any cattle in a herd have altered temperature and/or heart rate, and alert issues as to possible altered health status and early disease onset. These cows may be isolated and tested further for more detailed diagnosis. For example, farmer Joe Smith monitors the daily temperatures of all of his cattle, and one day is alerted about an increase in temperature of his cow #1105. In this example, a new disease among cattle is spreading, but the authorities are not yet aware of this disease. However, farmer Joe Smith alerts the authorities when, after a few days, the isolated cow dies of infection. The authorities then isolate all cows with the elevated body temperature located on other surrounding farms. Even though the veterinarians and authorities could not identify the pathogen, the disease is halted from spreading to other cattle and humans because the new pathogen is prevented from entering into the food chain. This scenario with the OSAC system of the invention makes possible the prevention of the deaths of both cattle and humans from diseases such as mad cow disease and brucellosis.

In another embodiment of the invention, parents were placed in a position where they had to leave in a hurry due to a family emergency. They left their child, Peter, with a new babysitter in the babysitter's home. Using the smart pacifier system of the invention, they activate the smart pacifier and it's appropriate accessories to remotely monitor their baby's health and well-being, including temperature, pulse rate, crying, feeding, sleep patterns, etc. through use of biosensors, cameras, microphones, etc. and can even comfort the child through, e.g., a camera, video, or audio on the smart-pacifier, smart phone, etc. and ensure that proper medications and nourishments are dispensed to their baby.

### Example #7

Mary is busy traveling for her job. Therefore, she misses her doctor's appointment. But her doctor sends her a packet with several biosensors and instructions to use them (according to the invention). The panel of biosensors sent is based on her medical history, her family medical history, and any diseases specific to her. Examples of biosensors included in the packet for use in the invention include blood chemistry sensors such as sodium, potassium, calcium sensors, blood glucose, lipid profile sensors, hemoglobin, hemoglobin A1C, blood creatinine, and C-reactive protein, temperature, heart rate, blood pressure, cancers that run in her family, common infections and allergens found in her environment, and any drugs that she might be prescribed at the time. She uses each biosensor as instructed; such as for example, blood glucose should be used during early morning fasting time, but the temperature one should preferably be used at all hours for over a few days. All values obtained are sent to her physician's office, and her physician may discuss her health status with her during her next visit.

7. Activation and Wake-Up by Another Biosensor, Device, and/or Remote Mode.

The OSAC system described herein includes biosensors located elsewhere within and/or on an animal or human. The OSAC system also interfaces with various other devices located on or near an animal or human, and may securely communicate with a plurality of remote devices that monitor the health and/or well-being of one or more animals or humans. Other diagnostic devices, drug-dispensing devices, other devices, and/or other biosensors are optionally used to activate or wake up the OSAC system of the invention. The OSAC system e.g., may also is optionally remotely woken up by a caretaker, healthcare-provider, and/or others. In yet another embodiment of the invention, the described OSAC system triggers activation and wake-up. These triggers for example, in certain protocols, includes the oral cavity's tongue/teeth/finger(s), etc. and may be self-induced in order to control and activate one or more biosensors within the oral cavity in conjunction with remote activation through one or more smart devices. Local, regional, domestic, or international monitoring and notification may be transmitted through the OSAC system of the invention. Further, many examples of embodiments of this invention are possible with several different biosensors and devices for both humans and animals without departing from the scope of the invention. In addition, the OSAC system of the invention includes storing secure data captured by the oral cavity when no connectivity is available. The OSAC system of the invention both stores and/or stream data and/or packeted as programmed. In addition, if OSAC-captured data is securely stored within the mouth cavity or in close proximity to the biosensors, it is optionally streamed when connectivity becomes available. In addition, audio, video or any form of enriched data (multimedia) is activated when necessary for security purposes and protection. The activation of both biosensors and data capture (camera, mic, etc.) is optionally hidden within the oral cavity and/or is activated in a stealth manner, using techniques known to those familiar with the art. The camera is optionally activated to take pictures of abnormalities in the oral cavity during periods of pain, inflammation, pus discharge, etc. These images help a healthcare provider determine and treat oral diseases remotely or as an out-patient.

An example of a diagnostic device that may be used in this system, a cardiac monitor is employed to detect abnormalities. When it does so it sends a signal to activate the described OSAC system to begin monitoring and collecting data about the patient's electrolyte imbalances, blood pressure, and/or prescribed drug blood concentrations. The cardiac monitor also sends an alert to the healthcare provider who may remotely activate additional biosensors within the OSAC system. Another example of drug-dispensing device activation involves when a cisplatin-dispensing pump activates the OSAC system to monitor blood pressure, blood oxygen levels, heart rate, and/or blood cisplatin concentrations. If one or more of these values are sufficiently different from normal preset values, the OSAC system may send an alert to the drug-dispensing pump to alter the amount of drug being administered. An alert is also optionally sent to the healthcare provider so they take any appropriate action. In this embodiment, the OSAC system of the invention may communicate with medical devices, a drug dispenser pump, and nano-particles as to the medication for accurate diagnosis and treatment.

### Example #8

Bogie's owner remotely activates an OSAC system as described to see Bogie's stress levels, his current state, and his biomedical conditions, infections, dehydration, etc. If the dog has to go to the bathroom to relieve his stress, the master instructs the dog over two-way communications, through a camera and speaker, as an option to go to bathroom.

Examples of other biosensors located within and/or on a human or animal, which triggers the described OSAC system, are smart pills and miniaturized cameras. A biosensor or nano-particles released from a smart pill by stomach acid activates the described OSAC system. For example, this released biosensor interfaces with the described OSAC system to send an alert to begin blood pressure monitoring. It also sends an alert to the healthcare provider so that they can take any appropriate action.

8. Communication Through WiFi and Other Wireless Modes.

In this mode, WiFi, GPS, GPRS, or other secure communications occur between the described OSAC system and sensors located elsewhere within and/or on an animal or human. The OSAC system also securely communicates through WiFi, GPS, GPRS, or other methods with other devices located on and/or near an animal or human. These communication modes are subject to a plurality of remote devices that monitor the health and/or well-being of one or more animals or humans. Local, regional, domestic, or international monitoring and notification may be transmitted through the described OSAC system. When these communications are lost, the data is stored locally until the communication is restored and the data is streamed to a central secure server. Many examples of embodiments of this invention are possible with both humans and animals, and the ranges and preset values discussed here are used only as examples without limiting the scope of the invention.

### Example #9

Bogie's owner routinely leaves Bogie home alone for several hours and wants to ensure his well-being. In this example, a breed-specific kit is provided for Bogie. The kit contains one or more oral biosensors that are breed-specific (he is a Great Dane) to diagnose his overall health and disease status, including his stress status, heart rate, and other canine-related issues. Bogie's collar or another accessory attachable to his collar, on Bogie's body, and/or other accessories contains a built-in WiFi and/or GPS, GPRS, microphone, speakers, two-way cameras with video capability, and sound sensors, video camera, an optional mobile device, and a treat dispenser. This embodiment reflects a combination of multiple communication modes for the described OSAC system. The OSAC system as described is programmed to become automatically activated when a disease or elevated stress levels are detected as previously exemplified. In addition, the OSAC system is activated remotely by the owner, caregiver, and/or healthcare provider and accessed through one or more servers. The GPS XYZ coordinate mapping maps the pet's indoor positioning when alone and also optionally is activated if Bogie wanders off into areas of his habitat or living space that are set as off-limits, for example. Once activated, alerts are sent to the owner and/or healthcare provider; the collar's WiFi device optionally communicates with one or more mobile devices such secure servers, laptops, PCs, iPad, and other mobile devices. The OSAC system of the invention also optionally transfers voice commands and other information to speakers, two-way speakers, and video cameras on the collar that will activate a plurality of devices. In addition, a treat dispenser may be remotely triggered for Bogie. The owner may interact with Bogie in real-time and through video cameras, microphone, speakers, and via a treat dispenser. The owner may instruct Bogie to perform tasks to reduce and monitor all forms of metabolic conditions including, but not limited to, stress, infections, heartbeat, etc. In addition, the described OSAC system not only reduces stress, but also detects when Bogie needs to be let outside in order to urinate or defecate, which will reduce stress to a trained animal. All the data collected by the OSAC system is sent to a secure server that is remotely accessed by the healthcare provider(s) and the owner(s). Each episode is optionally compared to Bogie's previous recorded history, the history of similar dogs in similar locations or circumstances, or even to other breeds of dogs, animals, and/or humans, as applicable. The owner and/or healthcare provider accesses the overall medical history of Bogie, his historical analyses, and other comparatives with dogs of the same or different breeds. The OSAC system as described may be used for any pet of any breed for any metabolic and situational uses. The OSAC system also enables oral sensors the ability to interface other devices located on or near an animal or human, and securely communicate with a plurality of remote devices that monitor the health and/or well-being of one or more animals or humans.

Anyone familiar with the arts understands that the above example can be replaced by a baby alone with a babysitter. This mode could be used to monitor a baby's health and well-being by monitoring the child's feeding, sleep, crying, and/or movement in the house, her other vital metabolic conditions, etc. through a smart pacifier. Once activated, alerts can be sent to the parents and/or other authorized people; the pacifier's WiFi device may optionally communicate with one or more mobile devices, such as secure servers, laptops, PCs, iPad, and other mobile devices. The OSAC system of the invention also optionally transfers voice commands and other information to speakers, two-way speakers, and video cameras on the pacifier or its accessories that will activate a plurality of devices. In addition, a medication dispenser accessory of the pacifier may be remotely triggered for the child if the caretaker is not authorized to dispense the medication. The parents may also interact with the baby in real-time and through video cameras, microphone, speakers, etc. using this aspect of the invention.

9. Concentration Alert.

In this mode, a component from salivary fluids, blood supply in oral cavity and/or lymph nodal fluids is optionally concentrated; when preset sufficient amounts of the said component is concentrated, an alert is sent and the biosensor along with the said component may be removed for further analysis. Encapsulated or un-encapsulated biosensors may act as an isolation and/or concentrating device with alert and communications sent at appropriate times. These communication modes are subject to a plurality of remote devices that monitor the health and/or well-being of one or more animals or humans. Local, regional, domestic, or international monitoring and notification may be transmitted through the OSAC system of the invention. Multiple examples of embodiments of this embodiment of the invention are possible with both humans and animals, and the ranges and preset values discussed here are used only as examples.

In one example of this embodiment of the invention, a biosensor is unable to determine the exact sub-type of cancer cells present in saliva or oral blood supply. Biosensors for all types of tumor, which represent 0.01% of all cells in the saliva, is installed on a retainer in the oral cavity. The biosensor binds to several types of tumor cells based on cell-surface biomarkers and only 10% of these tumor cells are malignant and cancerous. Therefore, saliva might contain 0.001% or less of malignant cells needed to diagnose the cancer. To collect ten microliters of the malignant cells for analysis in the laboratory, 100 milliliters or 0.1 liter of saliva might be needed. However, these cells do not survive for too long outside of the body; so when outside of a human body, these cells need to be stored properly at subzero temperatures. Due to these conditions, it is difficult to diagnose these cancers accurately until malignant cell concentrations in the saliva increases; however, when more such cells are present in the saliva, the disease could have progressed and could lead to poor prognosis for the patient since by then the disease might have advanced and be difficult to treat. However, according to this aspect of the invention, the biosensor is allowed to bind to the tumor cells for several days to weeks, whereby a sufficient preset amount becomes concentrated to properly, reliably, and timely diagnose the cancer. When a sufficient preset amount binds or absorbs to the biosensor, an alert is sent to remove and send the biosensor or biosensor data acquired for further analysis. This situation can be used according to the invention wherein it is substituted by other infectious diseases or other diagnostic molecules that are initially present at sub-diagnosable amounts.

In another example of this embodiment, still undiscovered, specific RNA is a predictor of a certain metabolic disease. However, the amount of this RNA in saliva and/or blood is so minuscule that it makes the RNA identification difficult or even impossible, so the disease prognosis is poor. In this aspect, the biosensor of the OSAC system of the invention concentrates this RNA over the course of several hours to days or weeks until the RNA in question is sufficiently concentrated and an alert is sent to remove the biosensor, or to send its accumulated data, for further analysis. This ability to concentrate might not be practical by commonly practiced RNA concentration methodology from saliva or blood due to the miniscule amount of RNA present in the saliva or blood. The RNA containing biosensor according to this aspect of the invention can then be sent, or its accumulated data sent, to a laboratory for analysis, for example, by amplification, sequencing, etc. This entire process makes diagnosis more reliable and accurate.

In a further embodiment, the biosensors are installed in parts of a pacifier configured to go inside the oral cavity or which are in contact with oral cavity components such as lips, cheeks, gums, etc. A polarity of RFID readers, audio microphones, cameras, etc. can also be installed on a pacifier and/or on any accessory, bracelet, toy, blanket, rag, bib, clothing, smart phone, etc., or on or in or around a baby. In an embodiment, the nipple of the pacifier contains biosensors inside the nipple, on the inner surface, on the outer surface or on a combination of one or more, where these biosensors assist in the collection and concentration of biologics and biologically relevant molecules. After preset amount of the materials are collected, an alert is bi-directionally transmitted to the parents, legal guardians, healthcare providers, etc. to allow them to take appropriate action. A similar device can also be used for adults and animals.

In one example of this embodiment of the invention, exposure to infections is determined. Peter is a 4 month old infant, and he spends his days in a day-care-center while his parents go out to work. His parents get a notice from the day-care-center about the spread of an infectious disease. His parents replace his pacifier with another one that contains biosensors for detecting the infection using an OSAC system according to the invention. When enough infection-detecting material has been collected in the pacifier, an alert is transmitted to the parents, legal guardians, healthcare providers, etc. The materials collected and concentrated in the pacifier may be further tested to establish that Peter is infected and then treatment is provided for the infection.

10. Other Combinations Using the OSAC System.

In addition to the above, any and all combinations could be exemplified for a multitude of OSAC purposes and uses. An auto-mode, manual mode, etc. pertain to multiple OSAC applications. Local, regional, domestic, or international monitoring and notifications may be transmitted through the OSAC system. Additionally, many examples of embodiments of this invention are possible with both humans and animals, and the ranges and preset values discussed here are only used as examples.

### Example #10

This example may be utilized for both human and animal patients Bogie is routinely left home alone and his owner wants to ensure his well-being. This embodiment reflects a combination of all communication modes for described OSAC system. The utility of this example of an embodiment in WiFi modes is previously explained in detail. The OSAC system is programmed to become automatically activated when a disease or elevated stress levels are detected as previously exemplified. The OSAC system of the invention may be used for any breed, pet, or for farm animals, such as, horses and cattle. Further laboratory animals could be subjects of the species-specific embodiment of the OSAC system. The OSAC system is optionally used for any metabolic disease, other disease, and/or situational use; so this embodiment can be disease and/or situational specific. The owner, caretaker, and/or another person, remotely or otherwise, activate this embodiment in manual mode. The OSAC system of the invention enables oral sensors to interface with other devices located on or near an animal or human, and securely communicate with a plurality of remote devices monitoring the health and/or well-being of one or more animals or humans. Thus, this embodiment can be used as a device or another biosensor with remote activation or wake-up mode settings. Caretakers, healthcare providers, etc. can visualize data such as, but not limited to, correlations between different episodes and historical data for Bogie, any other animal, or human within the same species, breed, or disease category, or within different species, disease categories, or situations.

In another embodiment of this invention, biosensors measuring many biostats, such as temperature, heart-rate, breathing rate, blood oxygen levels, muscle tone, etc. are installed in a pacifier similar monitor which is used for babies, adults, or animals. The biosensors are installed in parts of the pacifier that are inserted inside the oral cavity or are in contact with the oral cavity or components, such as lips, cheeks, gums, etc. A polarity of RFID readers, audio microphone, camera, etc. are also installed on the pacifier and/or on any accessory, bracelet, toy, blanket, rag, bib, clothing, smart phone, etc. or other sensor receptacles on, in or around the baby. In this embodiment, the pacifier also functions as a prescription or non-prescription medication dispensing unit that dispenses medication from the pacifier. The dispensing can be operated automatically, manually, or some combination. The dispensing unit can hold or dispense pre-measured amounts of the medication. The dispensing unit can be calibrated so that correct amounts of medication can be accurately dispensed or it can be configured to contain pre-measured amounts of the medication. In an embodiment of this invention, for example, Peter, from our earlier example, is only 4 months old and spends his daytime in daycare center while both his parents work. Peter develops a fever due to a bacterial infection in his lungs; his physician prescribes an antibiotic which alleviated the symptoms. After a few days of rest with his parents at home, Peter needs to go back to the daycare center because his parents need to return to work. Peter is still susceptible to contacting the influenza virus or a common cold, so his vitals must be measured frequently. His mother gives him an alerting pacifier with biosensors and a drug-dispensing unit to his caretaker, and his medications. The medications may be pre-measured and sealed doses of physician recommended, non-prescription, and/or prescribed medications which comes in a sterile, disposable, or non-disposable pacifier nipple, cartridges, bags, sacks, etc. Parents can either buy pre-packaged medication based on the child's age, weight, etc., or they can fill and seal the pacifier nibbles, cartridges, etc. themselves, based on physician recommendations. The medication-filled pacifier nipple, cartridges, etc. can also have RFID and finger-print and/or password numbers so that the medication can be linked to Peter for the specific date and treatment employed, and the password/fingerprint prevents accidental dispensation. In this case, e.g., a pain-reducing, non-prescription medication used to reduce fever, may be used. In this embodiment, an alert is bi-directionally transmitted every four hours to the caretaker to ensure that the pacifier is in Peter's mouth, so caretaker ensures that Peter uses his pacifier every 3-4 hours which is sufficient to pacify him. Peter's parents and physician, e.g., get alerts regarding Peter's biostats, and if his temperature exceeds 100° F., he must be administered a fever-reducing medication, such as Tylenol®. Because babies and small children are unable to communicate their physical discomfort, such as feeling the onset of fever, aches and pains due to infections and other diseases, this alerting thermometer in pacifier form provides both a very early means to inform parents and physicians about the health of the child before obvious signs and symptoms develop, and also serves to comfort and pacify the child. When an alert of an increase in core body temperature is bi-directionally transmitted to the parents, they may call to ensure that the medication was given to their child. The system also permits them to expressly consent, give approval, watch, verify, sign, authorize by password, etc. the specific medication being administered to the child over bi-directional camera and audio systems made part of the pacifier or other systems around, and providing support for, the child. Alternatively, the parents can remotely dispense the medication themselves in the presence of caretaker, which is useful when the caretaker is unable to do so for various reason including, but not limited to, limited legal authorization. When the next monitor reading is normal, they know that the medication worked well. In a further embodiment, this scenario also plays out where the baby is in the hospital or in the care of friends and family other than his parents or legal guardians. Parents or legal guardians can use this device without the necessity for obtaining remote consent, approval, etc. Depending upon need, this device can be used in several different modes or a combination of more than one. This device or similar devices, smart thermometers, a smart gauge, smart dipstick, smart rod, smart stick, smart device to collect blood or saliva, etc. may be used for human and animals.

In another embodiment of this invention, biosensors to measure many biostats such as temperature, heart-rate, breathing rate, blood oxygen levels, muscle tone, etc. may be temporarily installed in the oral cavity. These can be installed contemporaneously with the beginning of a new medication regimen. In fact, the biosensors can be installed before the start and during continuous use of a drug course. These biosensors can measure the overall health and impact of a new medication on an individual, because the biosensors concomitantly measure the biostats during the administered drug course. In certain embodiments, biosensors to measure the drug-blood concentration can also be installed to provide even more accuracy. The alerting system for this can be set up to provide real-time, near-time, manual, or automated preset values. In certain embodiments, the patient, caretaker, or the healthcare provider can visualize a graphic representation of this bio-feedback data to determine its effectiveness, efficacy, and side-effects profile for the individual and compare it with pre- and post-drug administration data, as well as with other individuals of varying age, gender, size, ethnicity, etc. in the population. This may help physicians tailor medications for individual needs, and thus, assist in personalizing the practice of medicine and help bring down the cost of healthcare. This model can be used in clinical trials and can track adverse events and side effects detected by biosensors or diagnostic devices. Further, the limited information obtained may be entered in real time by the patient. For example, Mr. Smith, diagnosed with a chronic sleep disorder and a heart arrhythmia, needs to start a medication that could aid in him getting enough sleep. His inability to sleep causes stress-related cardiovascular symptoms which may precipitate his arrhythmia. Accordingly, his physician decides to put him on a medication to help him sleep. The physician has several options because several different medications are available in the market, but the physician wants to ensure that the prescribed drug does no harm and is an effective sleep aid for this patient. In this scenario, the physician puts Mr. Smith on an OSAC system according to the invention before the drug is prescribed with biosensors for high blood pressure, heart rate, holter monitor, core body temperature, cortisol, and alpha-amylase. Heart function can be determined by biosensors for blood pressure and heart/halter monitor; whereas, lack of sleep can be determined by biosensors for core body temperature, cortisol, and alpha-amylase. After a few days on the OSAC system, Mr. Smith is prescribed a sleep aid drug. However, this drug is ineffective and the reporting from the OSAC system remains unchanged. After two weeks, the physician changes the drug and the OSAC system suggests that the drug is effective but Mr. Smith's alpha amylase levels are still slightly elevated which is suggestive of inadequate sleep, so the physician increases the drug dose. After two weeks Mr. Smith's biostats becomes normal. The OSAC system helped the physician quickly find the appropriate drug and dose fit for the patient. That result in turn may help reduce emergency room visits because Mr. Smith's untreated sleep disorder could have precipitated his ongoing cardiovascular disturbance. Moreover, a suitable drug was prescribed that did not adversely impact his overall health and potentially precipitate other diseases.

In still another embodiment of this mode of the invention, biosensors can be used to determine fertility, ovulation, and pregnancy. The OSAC system can be set to a combination of automated, real-time or near-time and manual modes. It is understood by anyone familiar with the art that in females, basal body temperature and several hormones associated with menstrual cycles and pregnancy change during the menstrual cycle. For example, during the ovulation phase of the menstrual cycle, follicle stimulation hormone (FSH) and Leutenizing Hormone (LH) from the pituitary glands are elevated. Basal body temperature is slightly elevated, for example, from 97.0° to 97.5° F., just after ovulation has occurred. This signals that a woman is most fertile just before such an increase in temperature. Following increases in basal body temperature and changes in cervical mucus over a couple of menstrual cycles can help women determine their optimum fertility timings. This information may be used by many women to either become pregnant or to avoid becoming pregnant. If the basal body temperature does not decrease after about 14-16 days, pregnancy could have occurred. This is the quickest way to determine if pregnancy has occurred. During pregnancy, the pregnancy announcing hormone, human chorionic gonadotropin (hCG), appears elevated in the blood and during labor several hormones, including oxytocin, adrenaline, and endorphins become elevated. Biosensors for temperature and various hormones may help in diagnosis of the onset of ovulation, pregnancy, labor and some diseases related to fertility. Accordingly, incorporating such bio-sensing indicators into the OSAC system of the invention provides for the benefits delineated above.

In another embodiment of the invention, race horse, Swale, is a prized stallion of his owner. Swale's owner can use the OSAC system to ensure that the stallion is properly trained, is in good health, and to compare his biostats with other race-winner horses. Biosensors can be attached to the horse's bit; whereas, his rein, saddle, etc. can become smart devices which communicate with the OSAC system and other biosensors on, in, or around the horse. The veterinarian can monitor Swale's overall health by utilizing an automated mode for biosensors for temperature, heart rate, respiration rate, electrolyte balance, etc. Unknown to the owner or the veterinarian, Swale could develop a temporary low-grade infection which can impact his performance during a very important race. To avoid such a situation, the owner uses an OSAC system to monitor the stallion's daily health and well-being. During training, the owner can compare biostats from Swale to others which helps the trainer to make better educated decisions about the proper training for Swale and other race horses. During training and races, the owner can monitor the horse's biostats and his performance by using additional sensors such as inertia, accelerometer, gyroscope, etc. in real-time or near-time modes.

In another embodiment of the art, Bogie's owner added two more dogs, one younger and another smaller, and one cat with a limp to his family. All four pets and the owner have an OSAC system. Every day all of them, the three dogs, a cat, and the owner, go for a jog or brisk walk. In this embodiment of the invention, the OSAC systems can enable the owner, for example, to help ensure that the exercise is optimizing everyone's health/goals. Results from the OSAC system for one or more exercise cycles can help him determine behavior patterns, through biosensor and wearable measurements, that are best for achieving and optimizing the overall health for his pets and for himself. These individual and comparative data analyses can help him to make important decisions about safe and healthy exercise for them individually and collectively.

In another embodiment of this invention, the dog, Bogie, had surgery and is very susceptible to getting infections. His veterinarian wants to keep a close watch on his biostats, such as his core body temperature, pulse rate, blood oxygen levels, etc., so the veterinarian installs an OSAC system in Bogie's oral cavity. The veterinarian wants the OSAC system to measure Bogie's core body temperature and blood oxygen levels every 2 hours, but his heart rate must be monitored constantly. However, Bogie likes to ingest warm food and milk, so to prevent unnecessary and false alerts, whenever the dog eats or drinks, the owner either puts his core temperature part of the OSAC system to sleep or stops it for the duration of the activity. Bogie's pulse rate increases when he goes for a walk every morning and evening, so the veterinarian could put two preset values for the heart rate, one for at rest of 218 to 225 beats per minute, and the other to monitor activity of from 218 to 240 beats per minutes. Therefore, when the owner takes Bogie for a walk, he can change the preset pulse rate values from rest to activity to prevent false alerts being transmitted. Furthermore, the core body temperature presetting could be set for two levels, a first level of an elevation from 101° to 103° F. for 12 or more consecutive readings could transmit an alert of low grade fever, and a second level, for one or more readings of 104° F. could transmit an alert of incipient high fever. The veterinarian could then treat Bogie differently when he gets the two different alerts.

According to embodiments of the invention, different tier, degree, and intensity levels of alerts are possible with each one of the alert modes. Basal measurements for any diagnostic parameter can be set at the lowest level of alert for each mode. Some alerts can be established as repetitive alerts, and bi-directionally transmit out alerts at pre-set interval until alert-receipt is acknowledged by the receiver.

OSAC-related biomarkers and applications: these oral biosensors optionally are used for a variety of disease-related biomarkers including, but not limited to, oral and systemic infectious diseases, cancers, drug abuse, metabolic diseases, malnutrition, obesity, cardiovascular diseases, atherosclerotic inflammation, stroke, and still-to-be discovered disease-signature and disease-linked biomarkers. While all of these and other applications, with a variety of oral biosensors, would yield significant information, the information without the communication and alert systems would not be nearly as useful to human and animal patients, caretakers, healthcare providers, and or others. This communication and alerting system, along with the oral biosensors, are incorporated into the invention OSAC system. In addition to the oral biosensors, the OSAC system of the invention includes other biosensors that interface with the OSAC system, and are located elsewhere, within, and/or on an animal or human. The OSAC system is capable of interfacing with a variety of other devices and nano-particles that are located in or on or near the animal or human, and can securely communicate with a plurality of remote devices that monitor the health and/or well-being of one or more animals or humans. The OSAC system of the invention also includes secure data system software with visualization modes, and presentation system software. The latter feature is available to utilize a variety of platforms including, but not limited to, charts, graphs, histograms, and/or bar graphs. Caretakers, healthcare providers, etc. can visualize data including, but not limited to, correlations and comparisons between different episodes and historical data for patients within the same species, breeds, and/or disease categories; or within different species, disease categories, and/or situations. For example, data from laboratory animals can be correlated and extrapolated to other animals or humans suffering from the same or similar diseases. Such kinds of scenarios are possible because the secure data system contains data captured and/or collected from various alerts. The secure data system also inputs data into the system in order to set alerts, which can be analyzed and visualized by the OSAC system software. Some of this data will be viewable and available to everyone, so will be public, yet some data will be viewable and available only to relevant individuals and healthcare providers, and hence will be private. The secure data system also inputs data into the system in order to set alerts, which can be analyzed and visualized by the OSAC software according to this aspect of the invention.

Divergent applications are possible with these biosensors. A few examples are transdermal, oral bleeding, or bathing in salivary secretions, bathing in lymph node fluid, and/or bathing in blood supply. The device may be present in an oral cavity for varying amount of times. The device is optionally part of a plethora of dental devices and/or it can be implanted flexibly, adhered, fixed, and/or cemented to the floor of the oral cavity. Specifically, the device is securable inside of or affixed to the cheeks, tongue, sublingual cavity, and/or other places within the oral cavity. The device locations within the oral cavity are optionally chosen by the patient, caretaker, and/or healthcare provider based upon convenience, comfort, need, duration of placement, durability, and other factors. The biosensor might be placed by a healthcare provider including, but not limited to, physician, surgeon, dentist, veterinarian, healthcare provider assistant, caretaker, and/or the patient him- or herself; the placement might be done, e.g., under mild sedation, full sedation, local anesthesia, general anesthesia, or other.

Saliva is secreted by salivary glands, which are made up of Acinus cells. Acinus cells are highly vascularized cells that cause blood constituents to enter saliva and secrete salivary juices into the oral cavity. Changes in the molecular composition of the blood are consequently reflected in the salivary secretion composition. Therefore, disease-specific blood-based biomarkers in salivary secretions depict the biomarkers of the entire body and systemic system. These biomarkers are also used to diagnose and monitor systemic diseases. Often these biomarkers precede disease developments in both humans and animals; thus, these biosensors help determine the overall health statuses of both animals and humans. The oral cavity is unique in preventing several disorders or treating them in the early disease stages. Thus, this invention can help save billions of dollars in healthcare costs and increase both quality and quantity of life.

Saliva contains biomarkers that predict several diseases and overall health status of an individual. Biomarkers are found in blood, lymph node fluids, and salivary secretions, but some of them are exclusively present in salivary secretions. The National Institute of Health (NIH) defines a biomarker as any objective, measurable, and evaluable indicator of normal biologic processes, pathogenic processes, or pharmacologic responses to therapeutic interventions. A biomarker that is reproducible, reversible, and reliable and may be associated with a particular disease is a signature of that disease. A variety of molecules can be biomarkers (see Table II for examples) including, but not limited to, proteins, immune response molecules like immunoglobulins and antibodies, nucleic acids like DNA and RNA, lipids, metabolites, histones, modified proteins and nucleic acids, and microbes. Alterations in signature biomarker concentration, structure, function, and/or action could be associated with the onset, progression, or even regression of a disease associated with the signature biomarker. Therefore, the biomarker is a valuable tool in the detection, diagnosis, risk-assessment, prognosis, and monitoring of a disease. Besides the biomarkers mentioned in Table II, several more are present within the oral cavity. Recently, more than 3,000 species of mRNA and over 300 species of miRNAs were found in the oral cavity. These discoveries offer unique opportunities to diagnose the onset of several diseases before the disease symptoms become apparent, since mRNAs must be synthesized or transcribed by a cell before proteins are translated or synthesized from them. Protein formation precedes physiological events that are observed as symptoms of a disease.

A major entry point for pathogens, allergens, food, drugs, and toxins is the mouth. Therefore, the OSAC system of the invention offers a unique opportunity to detect, diagnose, monitor, analyze, and assess the entry of these pathogens, drugs, and toxins into the body. Biosensors that detect these molecules are also incorporated into the OSAC system of the invention. The pathogens include viruses, bacteria, and other microbes that enter body through the mouth and nose. Pathogens enter the body through the mouth when these pathogens are present in airborne droplets, contaminated food, or the contaminated surfaces of the mouth and lips (via activities such as touching hands or kissing). SARS, tuberculosis, hantavirus pulmonary syndrome, and influenza are examples of airborne droplet transmission of respiratory system diseases. E. Coli and Salmonella enter the oral cavity via contaminated food going into the digestive system and cause digestive diseases. Finally, measles is an example of a systemic disease where the pathogen enters the body through the oral cavity, etc.

OSAC oral sensors of the invention also offer a unique opportunity to detect, measure, and monitor drugs and toxins. Drug and toxin consumption such as alcohol, or other oral prescription or non-prescription drugs may be determined. These sensors offer a unique opportunity to constantly and/or intermittently monitor drug and alcohol abuse. These are invaluable in the treatment and prevention of drug abuse and even in the reduction of car accidents due to driving under the influence of alcohol. Detection of alcohol in the oral cavity enables the OSAC system as described herein to send signals to a person's car. The OSAC system optionally signals the steering wheel to lock up when the drunken individual is sitting in the driver's seat, but, e.g., does not signal for lock up when the intoxicated individual is sitting in the back passenger seat. Similarly, another use is preventing narcotic prescription drug abuse. In this embodiment, real-time detection of ingested prescription medicine activates the monitoring of the time and amount of the drug consumed by the prescriber. Therefore, the OSAC system of the invention can prevent over-use or, in certain circumstances, even prevent the illegal sale of prescribed medicines. Therefore, the OSAC oral sensors may optionally also function as pill trackers.

The OSAC system of the invention is designed to monitor environment toxins, pollutants, and allergens all of which could be solids, liquids, or gases that enter the body through the mouth or nose. These may be used by soldiers in war zones when they are exposed to poisonous and weaponized gases and other toxins; miners in mines when they are exposed to deadly gases and other hazardous environments; or regular people in polluted cities. For example, current allergy testing measures the blood immune response to allergens, but oral sensors could measure the exposure and immune response at the site of allergen entry and response to them—the oral cavity. This is a substantially more accurate, inexpensive, and painless diagnostic measure compared to the inaccurate, painful, and expensive current methods available.

Lymph nodes offer new, novel, and unique opportunities to detect several diseases where the immune response is the initial primary response, or where biomarkers are concentrated in the lymph fluid. Examples of such diseases are: infections, certain cancers, ingested pathogens, allergens, and toxins. Oral secretions drain into three lymph nodes—the submandibular, submental, and tonsillar fluids, from the cheeks, the lower lip, gums, and the anterior tongue, drain into the submandibular node from the lower lip and mouth floor. Fluids from the tongue apex drain into the submental node. Fluids from the jugulodigastric drain into tonsillar lymph nodes. Biosensors implanted in these lymph nodes according to the invention help in early diagnosis, detection, risk-assessment, prognosis, and monitoring of a variety of diseases with immune responses, infections, and locations where biomarkers either concentrate or appear during early disease progress.

In another embodiment of the present invention, the OSAC system as described is used to help recognize nutrition-related diseases, and assists in diagnosing, monitoring, assessing, and analyzing them via oral biosensors. Examples are the diseases related to malnutrition in children, the elderly, animals, or weight loss in obese individuals. Ingested calories are optionally calculated by certain mathematical determinations known to those skilled in this art after the oral biosensors detect and measure ingested carbohydrates, lipids, and proteins. This assessment assists, e.g., morbidly obese individuals lose weight, or athletes gain muscle mass. Preventing malnutrition of micronutrients, such as vitamins and trace minerals, could also be achieved by detecting them in ingested food.

In addition, if OSAC-programmed data-points according to the invention are designed to measure calories consumed, measurements by biosensors during or after meal consumption are preferred. For example, individuals may be alerted by text when they consume above a certain amount of calories. This is particularly significant in the weight loss of a pet animal. The owner is able to restrict calories, or additional food made less tasty to the pet in order to reduce consumption. Oral biosensors could help determine the content of consumed food; therefore, the biosensors could help in obesity and malnutrition control. The oral cavity contains taste buds and abundant nerve endings, so biosensors that pertain to taste or other physiological processes that require nerves would be best suited for their detection. In this embodiment, compatibility of OSAC with other wearables and apps such Fitbit would add significant value to weight loss programs. Furthermore, the oral cavity contains the tongue, which is highly innervated and contains taste buds. Biosensors that detect activation of taste buds could be helpful in correlating food taste with activation of the taste buds. This in turn can be used to make some food more or less palatable, and hence, help in weight loss or in treating malnutrition, or in tracing and tracking ingested food or medications.

The described OSAC system is utilized, as described above, by animal and humans such as infants, children, disabled, or the elderly who are unable to communicate their health status to their caregivers. These individuals especially benefit from employment of these biosensors using the OSAC system of the invention. Besides predicting disease amongst animals, they optionally are employed in determining abuse. Abused animals are often malnourished, injured, and dehydrated; hence, malnutrition, injury, and dehydration, may be assessed, detected, diagnosed, and monitored for signs of abused animals.

Data collected from animals using the described OSAC system not only helps determine their health status, but also may optionally be extrapolated to humans. These animals could, in other words, constitute and serve as animal models for humans. Every disease has a panel of diagnostic testing, recommended by healthcare organizations such as Center for Disease Control (CDC) or Center for Medicaid and Medicare (CMM). Problems associated with diagnostic testing are major hurdles to early detection and effective treatment. Most diagnostic tests are expensive, time-consuming, invasive, and inaccurate. Therefore, researchers are constantly searching for ways to improve current diagnostic tests or new methods of diagnostic testing. Timely diagnosis is an important key to the proper treatment of any disease. Oral biosensors used according to the described invention offer new, innovative, and unique opportunities to diagnose, detect, and monitor diseases. The use of these biosensors assists in the prognosis and risk-assessment of multiple different kinds of diseases.

Biosensors are typically of three major types. The first type is invasive and is used to analyze biological material outside the body, such as anti-HIV antibodies used to detect HIV viruses in a blood sample in a laboratory setting. The second type is invasive, but the monitoring is done by the patient or caretaker at the site of blood draw. An example of the second type is the most popular way to monitor blood glucose levels among diabetic patients, by pricking fingers and using the blood droplet to measure glucose levels. The third type is non-invasive and is used outside body, such as a pulse oximeter blood oxygen level monitor used to measure blood oxygen levels. This type has very limited use because no bodily fluids that contain most of the biomarkers are used.

The OSAC system of the invention offers a timely, inexpensive, non-invasive or minimally invasive, and accurate diagnostic tool to monitor a variety of diseases and patho-physiological conditions. It eliminates costly, invasive, painful, and time-consuming diagnostic testing methods. Detecting pathogens at their point of entry, before even the infection starts, significantly improves a patient's infection prognosis, therapeutic intervention, survival rate, and recurrence. The invention's use of technologies incorporating wearable devices, devices capable of storing and analyzing large amounts of electronic data, and wireless batteries enhance the benefits provided by the OSAC system of the invention. Frequent and continuous monitoring of the large amount of data generated is readily maintained, analyzed, and handled by the inventive system and method. Moreover, the wireless charging utilized is beneficial when frequent or continuous monitoring is required.

It is to be understood that the above description is intended to be illustrative, and not restrictive. For example, the above-described embodiments (and/or aspects thereof) may be used in combination with each other. In addition, many modifications may be made to adapt a particular situation, method, system device or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claims.

We claim:

1. A system comprising a device configured to be inserted and securely attached in an oral cavity of an animal or human comprising a smart sensor receptacle for a sensor, the receptacle being configured to serve one or more functions within the animal or human's oral cavity without being swallowed, the device further comprising one or more sensors contained within or upon the receptacle, and at least one interface with a network configured to utilize the information obtained from the one or more sensors or from one or more platforms.

2. The system of claim 1 wherein the one or more functions of the device is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological or grinding protective function, and providing a recreational or sports function, health analytics, diagnostic analytics, performance analytics; integration of body sensors, health-devices, nano-particles, and sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision or analytics.

3. The system of claim 2 wherein at least one of the one or more functions of the device is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological or grinding protective function, and providing a recreational or sports function.

4. The system of claim 1 wherein the smart sensor receptacle is a pacifier and the one or more sensors comprises sensors for temperature, blood pressure, core body heart rate, levels of a predetermined biologic, chemical or medication or their metabolites, optimum breathing, oral air-flow, accelerometer readings, gyroscopes, inertia-sensors, tracking sensors, sensors with input from cameras, videos, microphones, and speakers within the pacifier's plastic nipple encasement, other parts of the pacifier or a pacifier component, face shield, or neck.

5. The system of claim 4 wherein the camera is configured to face away from the baby and is configured to be activated and inactivated remotely.

6. The system of claim 4 wherein the camera is configured to record and send out alerts when a predetermined stimulus occurs.

7. The system of claim 4 wherein the pacifier is configured so that sensor measurements are displayed on the pacifier.

8. The system of claim 4 wherein the system further comprises one or more auxiliary sensor inputs from one or more of a camera, video, microphone, speakers, accelerometers, gyroscopes, or tracking sensors.

9. The system of claim 8 wherein the one or more auxiliary sensor inputs is configured to securely transmit sensor data through Bluetooth, GPS or other wireless means to one or more networked smart devices.

10. The system of claim 9 wherein the one or more networked smart devices comprises a controlling device to be monitored and to alert of a change in diagnostics.

11. The system of claim 10 wherein the controlling device is configured to notify one or more additional networked devices configured to be sent bio-stats and alerts.

12. The system of claim 11 wherein the one or more additional networked devices configured to be notified comprises a lockable device configured to be unlocked when authorized by a controller.

13. The system of claim 12 wherein the lockable device comprises a locking mechanism configured to allow an unauthorized third party access to information or to disallow access to information.

14. The system of claim 11 further configured to transmit information which optionally is shared with other networked devices.

15. The system of claim 4 wherein the one or more sensors is configured to be insertable within or upon the pacifier and is configured to be interchangeable or securely affixed.

16. The system of claim 15 wherein the sensor is insertable and is configured to be securely affixed to the pacifier, and is further configured to be exposed to content within the oral cavity.

17. The system of claim 4 wherein the pacifier is configured to be protected by a thin layer of material or by an air pocket and outer layer.

18. The system of claim 4 wherein a wireless transceiver chip is built into the pacifier device enabling the device to send and receive data, commands and other information.

19. The system of claim 4 wherein the system is further configured to dispense medications.

20. The system of claim 19 wherein the pacifier is calibrated for enhanced accuracy of measurement of medications.

21. The system of claim 4 wherein the pacifier comprises an insert configured to be opened and dismantled.

22. The system of claim 4 wherein the system further comprises a secure clamp or other mechanical, electronic or magnetic lock.

23. The system of claim 4 wherein the system is configured to be remotely controlled.

24. The system of claim 4 further comprising a pre-calibrated liquid cartridge configured to contain one or more medications and to be insertable into the pacifier device.

25. The system of claim 4 wherein pacifier accessories are configured to be attached to a feeding bottle to dispense and monitor medications, and to monitor feeding patterns, feeding times, durations, amounts, or liquid temperatures.

26. The system of claim 4 wherein the pacifier is configured to work in conjunction with an auxiliary device not inserted into the oral cavity.

27. The system of claim 26 wherein the auxiliary device is configured to sense when an infant has taken a full or partial dosage, and dispense medication as directed by a controller.

28. The system of claim 26 wherein the auxiliary device comprises one or more of pacifier accessories, pacifier-straps, strap-buckles, clips, pacifier-holders, clothing, or a separate attachment for a camera, motion detector, tracking sensors, video, speakers, microphones, digital storage devices, or WiFi.

29. The system of claim 28 wherein the separate attachment is configured to be fully or partially attachable to and removable from clothing, a crib, a toy, a blanket, a baby's person, a feeding-baby bottle, or a caretaker.

30. A system comprising a device configured to be inserted in an oral cavity of an animal or human comprising a smart sensor receptacle for one or more sensors wherein the receptacle is selected from the group consisting of a horse-bit, a smart thermometer, a smart gauge, smart dipstick, smart rod, smart stick, smart device to collect blood or saliva, a receptacle configured to be inserted but not swallowed, a receptacle for babies or adults with biosensors and an RFID, micro- and nano-sensors, micro- and nano-electronics, micro- and nano-enabled energy harvesting, micro- and nano-energy storage, micro- and nano-devices micro- and nano-programmable processors, micro- and nano-memory devices, micro- and nano-integrated power management devices, micro- and nano-programmable hardware, micro- and nano-wireless devices with communication capabilities across multiple frequencies located in an oral cavity or integrated outside of the oral cavity, and a device configured to be inserted into an animal or human's oral cavity, but not swallowed, one or more sensors contained within or upon the receptacle, and at least one interface with a network capable of utilizing the information obtained from the one or more sensors, one or more platforms, or one or more auxiliary devices or body integrations.

31. The system of claim 30 wherein the smart sensor receptacle comprises a pacifier and the smart sensor receptacle is configured with WiFi connectivity, the system further comprises one or more sensors for temperature or oxygen levels, the system further comprises tracking and camera functionalities, and the system further provides an alerting signal when outside a pre-set range.

32. The system of claim 30 wherein the smart sensor receptacle comprises a full or partial retainer, the system further comprises a smart mouth guard accessory, the one or more sensors comprise sensors for temperature or blood oxygen levels, the system is further configured with WiFi connectivity and is configured to provide an alerting signal when outside a pre-set range.

33. The system of claim 30 wherein the smart sensor receptacle comprises a full or partial retainer or retainer-like device, the system further comprises a smart mouth guard accessory, the system is provided with full connectivity, full server access and is configured for an analytical processing capability comprising racing performance analysis.

34. The system of claim 30 wherein the receptacle comprises a retainer or retainer-like device, a smart mouth guard accessory which attaches and detaches to the retainer or retainer-like device, and the system is provided with data storage or full connectivity capabilities.

35. The system of claim 34 further comprising full server access and wherein the system is configured to analyze individual or team sports performance as it relates to various body components and sensors.

36. The system of claim 34 further comprising a digital storage device or full connectivity capability configured to analyze individual and team sports performance as it relates to various body component sensors for post-play analysis and review.

37. The system of claim 1 wherein the network capable of utilizing the information obtained from one or more sensors comprises one or more units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, analyzing historical or previous data from same or different individual or team, visualizing or presenting development platforms.

38. The system of claim 1 wherein the network comprises a system of tracking an animal, animals, human or humans' biomedical condition integrated with an analytical or predictive capacity to determine or estimate possible points of origin, routes of travel, current location, or proximity to specific events or locations.

39. The system of claim 1 wherein the system of tracking is configured to track lost animals, humans, babies or adults.

40. The system of claim 1 wherein the system comprises at least one auxiliary sensor receptacle selected from the group consisting of a horse-bit, a mouth-bit, a bit-guard, a lip-strap, a smart thermometer, a smart gauge, smart dipstick, smart rod, smart stick, smart device to collect blood or saliva, a receptacle configured to be sucked but not swallowed, a receptacle for babies or adults with biosensors, sensors, cameras, audio speakers, an RFID inside or outside of a mouth, micro- and nano-electronics, micro- and nano-enabled energy harvesting, micro- and nano-energy storage, micro- and nano-device, micro- and nano-timer, micro- and nano-programmable processors, micro- and nano-memory, micro- and nano-integrated power management, micro- and nano-programmable hardware, micro- and nano-wireless communication capabilities across multiple frequencies, sensors located in a mouth or integrated outside of a mouth, and an animal toy which is configured to be sucked but not swallowed.

41. The system of claim 40 wherein the one or more sensors comprises sensors for a biologic, a biologically relevant molecule, temperature, blood pressure, pulse rate, blood oxygen level, respiration rate, optimum breathing, oral air-flow, gyroscopic measurement, accelerometer measurement, or kinematics, and the network capable of utilizing sensor information is configured to have the information transduced, amplified, or processed and a signal from the network transmitted through a RFID tag to an RFID reader on an accessory attached to or associated with a horse, the information comprising heart rate, oxygen level, gyroscopic measurement, accelerometer measurement and inputs for inertia-sensor, tracking sensors, camera, video, microphone, or speakers.

42. The system of claim 40 wherein the system further comprises one or more sensors placed within or upon horse-related equipment.

43. The system of claim 42 wherein the horse-related equipment is selected from the group consisting of a headstall, headgear, an ear-pom, a blinker hood, a hackamore, a noseband, a cheese-band, a bridle, blinders, winkers, ornaments, phalerae, and sallongs.

44. The system of claim 40 wherein the information utilized by the network includes one or more values integrating sensor data received by the oral bit guard sensor from a central cardiovascular system.

45. The system of claim 40 wherein information utilized by the network includes one or more of measurements of performance, measurements of health, information obtained from biosensors, kinematics information, information obtained from cameras, information from sensors placed on blinker hood, nose-pieces, information from sensors attached to nose or other facial parts, information obtained from a heart-monitoring device, heart-rate, or respiration monitoring devices attached to horse equipment associated with the horse, information monitored by placing sensors on manure catchers, information from sensors attached to or associated with diapers attached to or associated with a tail or a tailbone of the horse, information from sensors attached to or associated with a leg, torso, neck, head or other part of the horse, information from instruments used to measure performance, information from accelerometers, gyroscopic measurements, or inertia-sensors, information received from an RFID tag reader, information from sensors attached to or associated with a smart horse-rein, information received from sensors attached to or associated with inanimate objects around the horse or information from other horses.

46. The system of claim 1 wherein the system comprises one or more network units configured to carry out a functionality selected from the group consisting of signaling bi-directional transmissions to a secure server through one or more of WiFi, Bluetooth, GPS, and NFC, temporarily storing information in the smart device, bi-directionally transmitting alerts to pre-selected devices or pre-selected personnel.

47. The system of claim 1 wherein the network comprises one or more RFID components.

48. The system of claim 1 wherein the network comprises one or more cloud applications.

49. The system of claim 1 wherein the network comprises a real-time or near-time slumber to alert mode.

50. The system of claim 1 wherein the network comprises a manual control diagnosis mode.

51. The system of claim 1 wherein the network comprises a programmed automated diagnosis mode.

52. The system of claim 1 wherein the network comprises a geographic analysis mode.

53. The system of claim 1 wherein the network comprises a species classification analysis mode.

54. The system of claim 1 wherein the network comprises a disease specific or situational alerting mode.

55. The system of claim 1 wherein the network comprises a function by which the one or more oral sensors is activated by another sensor, device or remote controller.

56. The system of claim 1 wherein the network comprises transmission through WiFi or other wireless mode.

57. A system comprising a device configured to be inserted in an oral cavity of an animal or human comprising a smart sensor receptacle for one or more sensors wherein the receptacle is selected from the group consisting of a horse-bit, a smart thermometer, a smart gauge, smart dipstick, smart rod, smart stick, smart device to collect blood or saliva, and other bio-sensors, a receptacle configured to be inserted but not swallowed, a receptacle for infants or adults with biosensors on one side and an RFID on the other side which is on the outside of a mouth, micro- and nano-sensors located in a mouth or outside of a mouth, and a receptacle configured to be insert but not swallowed within an animal's oral cavity, one or more sensors contained within or upon the receptacle, and at least one interface with a network capable of utilizing the information obtained from the one or more sensors, or from one or more platforms or one or more body integration wherein the network capable of utilizing the information obtained from the one or more sensors comprises one or more units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, comparative, historical or previous from same or different individual or team, data producing, visualizing or presentation development platforms.

58. The system of claim 57 wherein the network comprises a system of tracking an animal, animals, human or humans' biomedical condition integrated with an analytical or predictive capacity to determine or estimate possible points of origin, routes of travel or proximity to specific events or locations.

59. The system of claim 57 wherein the receptacle comprises a horse-bit, the horse-bit is configured with WiFi connectivity, the one or more sensors comprise temperature, oxygen levels or respiration rate, and the system is further configured to provide an alerting function when outside a pre-set range.

60. The system of claim 57 wherein the receptacle comprises a horse-bit with full connectivity, the system also comprises full server access and is configured for an analytical processing capability comprising racing performance analysis.

61. The system of claim 57 wherein the system comprises at least one auxiliary smart sensor receptacle not configured to be inserted in an oral cavity of an animal or human.

62. The system of claim 57 comprising a network configured to analyze one or more performance parameters of a team sport or group activity.

63. The diagnostic system of claim 57 comprising a network configured to analyze one or more performance parameters of horse racing.

64. The system of claim 57 comprising a network configured to provide an electronic medical records functionality.

65. The system of claim 57 wherein the network comprises one or more RFID components, micro- and nano-electronics, micro- and nano-enabled energy harvesting, micro- and nano-energy storage, micro- and nano-devices, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple frequencies located in a mouth or integrated outside of a mouth.

66. The system of claim 57 wherein the network comprises one or more cloud applications, a real-time or near-time slumber to alert mode, a manual control diagnosis mode, a programmed automated diagnosis mode, a geographic analysis mode, a species classification analysis mode, or a disease specific or situational alerting mode.

67. The system of claim 57 wherein the network comprises one or more medical devices or medication dispensers.

68. The system of claim 62 wherein the one or more medical devices or medication dispensers comprise a fully integrated treatment facility.

69. The system of claim 57 wherein the network comprises a function by which the one or more sensors is activated by another sensor, device or remote controller.

70. The system of claim 57 wherein the network comprises transmission through WiFi or other wireless mode.

71. The system of claim 57 further comprising at least one auxiliary smart sensor receptacle for a sensor.

72. A device configured to be inserted and securely attached in an oral cavity of an animal or human comprising a smart sensor receptacle for a sensor, the receptacle being configured to serve one or more functions within the animal or human's oral cavity without being swallowed, the device further comprising one or more sensors contained within or upon the receptacle.

73. The device of claim 72 wherein the one or more functions is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological or grinding protective function, and providing a recreational or sports function, health analytics, diagnostic analytics, performance analytics; integration of body sensors, health-devices, nano-particles, and sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision and analytics.

74. The device of claim 72 wherein at least one of the one or more functions is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological or grinding protective function, and providing a recreational or sports function.

75. The device of claim 72 wherein at least one of the one or more functions comprises replacing missing teeth or parts of teeth and the receptacle is selected from the group consisting of dentures, crowns, bridges, dental implants, permanent or temporary caps, fillings, fixed prostheses, prostheses, artificial teeth, prosthodontics, sealants, dental composites and bonds.

76. The device of claim 72 wherein at least one of the one or more functions comprises repairing broken teeth and the receptacle is selected from the group consisting of inlays, onlays and crowns, dental implants, permanent and temporary caps, fillings, fixed prostheses, prostheses, artificial teeth, prosthodontics, sealants, dental composites and bonds.

77. The device of claim 72 wherein at least one of the one or more functions comprises providing an aligning, fixing of malpositioned teeth or jaws, or other corrective function and the receptacle is selected from the group consisting of retainers, braces, space maintainers, headgear, palatal expanders, fixed prostheses, braces, dental wires, partial retainers or full retainers.

78. The device of claim 72 wherein at least one of the one or more functions comprises providing a cosmetic or cleansing function and the receptacle is selected from the group consisting of veneers, whitening and cleansing strips, and professional, amateur, or lay-person cleansing tools and equipment, and whitening or cleansing traces.

79. The device of claim 72 wherein at least one of the one or more functions comprises providing a pacifying function and the receptacle is a pacifier.

80. The device of claim 72 wherein at least one of the one or more functions comprises providing a physiological or grinding protective function and the receptacle is a night guard or partial guard.

81. The device of claim 72 wherein at least one of the one or more functions comprises providing a recreational or sports function and the receptacle is a tongue piercing, bendable or flexible sensory unit, electronic insert, stretch stripes, adhesive stripes, transdermal patches or tattoos.

82. The device of claim 72 wherein the receptacle is selected from the group consisting of permanent or temporary caps, implants, night guards, partial guards, crowns, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof.

83. The device of claim 72 wherein the receptacle is insertable by micro- and nano-clips, frames, brackets, sealants, dental composites, bonds, adhesives, adhesive strips, cements, wires, bands, glues, embedment, injection, printing, tattooing, or any combination thereof.

84. The device of claim 72 wherein the receptacle is configured to be removable.

85. The device of claim 84 wherein the receptacle is configured to be self-removable.

86. The device of claim 72 wherein the receptacle is configured to be self-installable.

87. The device of claim 72 wherein the receptacle is configured to be coverable with an air-tight material.

88. The device of claim 72 wherein the one or more sensors is integral to the receptacle.

89. The device of claim 72 wherein the one or more sensors comprises sensors of blood pressure, core body temperature, heart rate, optimum breathing, oral air-flow, levels of a predetermined biologic, chemical or medication or their metabolites.

90. The device of claim 72 wherein the one or more sensors measures a physical property.

91. The device of claim 72 wherein the one or more sensors measures a physical property selected from the group consisting of temperature, blood, pressure, teeth pressure, ionic conductivity, airflow, optimum breathing, oral air-flow, images, optical density, alterations to the oral cavity, surrounding muscle tone, muscle weakness, heart rate, heart rhythms, respiration rate, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, accelerometer activity, gyroscopic activity, kinematic activity and radiation wave activity.

92. The device of claim 72 wherein the one or more sensors measures a salivary, blood, lymph node, bone, or tooth constituent.

93. The device of claim 72 wherein the one or more sensors measures a predetermined biologic.

94. The device of claim 72 wherein the one or more sensors measures a predetermined biologic selected from the group consisting of DNA, RNA, telomeres, methylated or otherwise modified DNA or RNA, proteins, immunoglobins, antibodies, histones, peptides, modified proteins, neuro-peptides, pigments, and enzymes.

95. The device of claim 79 wherein the one or more sensors measures dissolved gases.

96. The device of claim 72 wherein the dissolved gases comprise oxygen, carbon dioxide, carbon monoxide, ammonia, sulphur, or an alcohol-containing gas.

97. The device of claim 72 wherein the one or more sensors measures a lipids profile.

98. The device of claim 72 wherein the one more sensors measures a chemical molecule.

99. The device of claim 72 wherein the one or more sensors measures a salt, an alcohol, a metabolite, an anion, a cation, water, a sugar, a protein, or a lectin.

100. The device of claim 72 wherein the one or more sensors measures a drug or a medication.

101. The device of claim 72 wherein the one or more sensors measures cells, cancerous cells, biomarkers for an oral or systemic infectious disease, biomarkers for drug abuse, biomarkers for a metabolic disease, biomarkers for malnutrition, biomarkers for obesity, biomarkers for a cardiovascular disease, biomarkers for atherosclerotic, biomarkers for infection, biomarkers for auto-immune and other immune diseases, biomarkers for stroke, biomarkers for AIDs, biomarkers for multiple sclerosis, biomarkers for periodontal diseases, biomarkers for brain-function disorders, dementia, memory loss, depression, mental disease, Alzheimer's disease, mentally-challenged disorders, nervous system disorders, tracking or wandering, and other psychology and neurological disorders, biomarkers for bleeding, head and neck injuries, biomarkers for Sjogen's syndrome, biomarkers for oxidative stress, biomarkers for allergies, biomarkers for cancer, biomarkers for skeletal and muscle diseases, biomarkers for genetic diseases, biomarkers for renal diseases, biomarkers for osteoporosis, biomarkers for fatigue, biomarkers for stress, biomarkers for sleep deprivation or sleep apnea, biomarkers for fertility, pregnancy, ovulation, and reproductive system disorders, biomarkers for cystic fibrosis, biomarkers for respiratory or pulmonary diseases, biomarkers for diabetes and ketoacidosis, biomarkers for inflammation, biomarkers for age-related diseases, biomarkers for dehydration, biomarkers for halitosis, biomarkers for alcohol consumption, alcoholism or drug consumption or drug addiction, biomarkers for hypoxia, smoking-related diseases, toxins, or pollutants, biomarkers for poor-gait, biomarkers for Crohn's disease, biomarkers for dental caries, biomarkers for blood and circulatory disorders, biomarkers for ear, nose, and throat diseases, biomarkers for taste, Ageusia, Hypogeusia, or Dysgeusia, biomarkers for bad-breath related diseases biomarkers for chewing or mastication, biomarkers for digestive disorders, biomarkers for hepatic diseases, spleen, gall-bladder and pancreatic diseases, biomarkers for urinary system disorders, biomarkers for integumentary system diseases, biomarkers for endocrine, lymphatic, and excretory diseases.

102. The device of claim 72 wherein the one or more sensors measures a cell surface component or a cellular marker or component.

103. The device of claim 72 wherein the one or more sensors measures a pathogen or a microbe.

104. The device of claim 72 wherein the one or more sensors measures administered foreign materials, medications, diagnostic molecules, drugs, biologically sensitive, derived, bio-mimics, or bioengineered molecules.

105. The device of claim 72 wherein the one or more sensors measures an ingested molecule or its metabolite.

106. The device of claim 105 wherein the ingested molecule is a pathogen, a microbial, an ingested toxin, or an ingested allergen.

107. The device of claim 105 wherein the ingested molecule is an ingested food constituent.

108. The device of claim 105 wherein the ingested molecule is a nutrient, a micronutrient, a fat molecule, a carbohydrate molecule, a sugar molecule, a protein molecule, or an amino acid.

109. The device of claim 72 wherein the one or more sensors measures an ingested medication, an ingested foreign material, an ingested drug, an ingested diagnostic molecule, an ingested biologically sensitive molecule, an ingested nanoparticle, an ingested derived molecule, a biomimic, or an ingested bioengineered molecule.

110. The device of claim 72 wherein the one or more sensors interacts with at least one disease-related biomarker.

111. The device of claim 110 wherein the at least one disease-related biomarker relates to a disease diagnosable by a sensor which is selected from the group consisting of sensors of blood pressure, core body temperature, heart rate, optimum breathing, oral air-flow, levels of a predetermined biologic, chemical or medication or their metabolites.

112. The device of claim 72 wherein the means by which the receptacle is securely attached within the oral cavity of an animal or human comprises one or more of being fixedly inserted, imbedded, fitted, fixed, implanted, fastened, joined, associated, coupled, linked, banded, united, mounted, combined, glued, adhered, cemented, or firmly connected by mouth parts or hands or accessories thereto.

113. The device of claim 72 further comprising an interface with at least one sensor or nanoparticles not located within the oral cavity.

114. A method for obtaining sensor data from an animal or human, the method comprising the steps of locating a device configured to be inserted and securely attached in an oral cavity of an animal or human, wherein the device comprises a smart sensor receptacle for a sensor, the receptacle being configured to serve one or more functions within the animal or human's oral cavity without being swallowed, wherein the smart receptacle contains or receives one or more sensors capable of providing or receiving information or analysis relevant to the animal or human, activating or monitoring the one or more sensors, and transmitting or receiving at least some portion of the information or analysis to, from or among a network or networks capable of utilizing the information or analysis.

115. The method of claim 114 wherein the one or more functions is selected from the group consisting of replacing missing teeth or parts of teeth, repairing broken teeth, providing an aligning, fixing-malpositioned teeth or jaws or other function, providing a cosmetic or cleansing function, assisting in proper breathing, eating or swallowing, providing a tongue thrust dental guard function, providing a pacifying function, providing a recreational or sports function, providing a physiological or grinding protective function, or providing a function related to health analytics, diagnostic analytics, performance analytics; integration of body sensors, health-devices, nano-particles, sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, or providing functions with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement or greater precision and analytics.

116. The method of claim 114 wherein at least one of the one or more functions is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological or grinding protective function, and providing a recreational or sports function.

117. The method of claim 114 wherein the information is transmitted securely to a plurality of remote devices monitoring the animal or human.

118. The method of claim 114 wherein the information is transmitted securely to a plurality of remote devices monitoring a plurality of animals or humans.

119. The method of claim 114 wherein the network capable of utilizing the information obtained from the one or more sensors comprises one or more network units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, comparative, historical or previous from same or different individual or team, visualizing or presentation development platforms.

120. The method of claim 114 further comprising the step of inserting the device and securely attaching it in the oral cavity.

121. The method of claim 114 wherein the network utilizes preset ranges, dynamic present ranges, or degrees of alerts from present ranges for medical or performance analysis.

122. The method of claim 114 wherein the network utilizes biosensor or sensor measurements for pre-integration and post-integration analyses.

123. A method for monitoring a drug treatment, medication usage, food consumption, exercise, behavioral, or other program or activity having a durational component, the method comprising monitoring at least one smart receptacle sensor located in an oral cavity of a human or animal, or at least one smart auxiliary receptacle sensor not located in the oral cavity but in communication with the oral cavity receptacle sensor, before, during or after the durational component is administered, consumed or undertaken, or some combination of such monitoring, thereby obtaining sensor data of one or more parameters of the durational component for transmission, analysis or both.

124. The method of claim 123 wherein the method comprises monitoring at least one parameter of a drug treatment or medication regimen before, during or after, or some combination thereof, administration of the drug or medication over a course of time.

125. The method of claim 123 wherein the method comprises monitoring at least one parameter of food consumption of a human or animal wherein the method comprises monitoring at least one parameter of the food consumption before, during or after or some combination thereof, consumption of the food over a course of time.

126. A diagnostic or other system for an animal or human comprising a device configured to be inserted and securely attached in an oral cavity of an animal or human, wherein the device comprises a smart sensor receptacle for a sensor, the receptacle being configured to serve one or more functions within the animal or human's oral cavity without being swallowed, wherein the smart receptacle is further configured to contain or receive one or more sensors capable of providing or receiving information or analysis relevant to the animal or human, the system further comprises a smart auxiliary device which is wearable, attachable or insertable externally to the oral cavity, wherein the auxiliary device is configured to obtain information from, provide information to, or both, the one or more sensors contained in, on, or received by the oral cavity receptacle, and the one or more oral cavity sensors or the auxiliary device, or both, are configured to transmit or receive the information or analysis to or from a network or networks.

127. The system of claim 126 wherein the auxiliary device is configured to contain or receive one or more sensors.

128. The system of claim 126 wherein the one or more functions is selected from the group consisting of replacing missing teeth or parts of teeth, repairing broken teeth, providing an aligning, fixing-malpositioned teeth or jaws, or other corrective function, providing a cosmetic or cleansing function, assisting in proper breathing, eating or swallowing, providing a tongue thrust dental guard function, providing a pacifying function, providing a recreational or sports function, providing a physiological or grinding protective function, or providing a function related to health analytics, diagnostic analytics, performance analytics; integration of body sensors, health-devices, nano-particles, and sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement or greater precision and analytics.

129. The system of claim 126 wherein at least one of the one or more functions is selected from the group consisting of replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological or grinding protective function, and providing a recreational or sports function.

130. The system of claim 126 wherein the animal is a pet.

131. The system of claim 130 wherein the pet is a dog or cat, or other animal.

132. The system of claim 130 wherein the auxiliary device is a smart collar.

133. The system of claim 126 wherein the system further comprises a historic database of the animal or human as to one or more characteristics from which comparisons or analyses are configured to be made.

134. The system of claim 126 wherein the system further comprises a database of animals or humans having a common characteristic to the animal or human on which the smart device is located and for which a predetermined comparison is configured to be made.

135. The system of claim 126 wherein the animal is a pet, the auxiliary device comprises a smart collar which connects via WiFi to an accessible portal and wherein the system provides diagnostic information regarding the pet.

136. The system of claim 130 wherein the system comprises a database compilation of one or more of the pet's biological or physiological attributes.

137. The system of claim 130 wherein the system comprises a database compilation of one or more attributes of animals having at least one similar characteristic to the pet.

138. The system of claim 126 wherein the auxiliary device comprises a smart device configured to be associated with one or more team members of a team wherein the auxiliary smart device comprises one or more sensors configured to obtain information from the one or more team members and configured to transmit the information or analysis derived therefrom directly or indirectly to a network.

139. The system of claim 138 wherein the auxiliary device is a mouth guard and the one or more auxiliary device sensors measures oxygen levels or heart rate.

140. The system of claim 138 wherein the network is configured to analyze fatigue.

141. The system of claim 138 wherein the network is configured to analyze the fatigue of an individual team member.

142. The system of claim 138 wherein the network is configured to analyze the fatigue of a composite of a plurality of team members.

143. The system of claim 126 wherein the network is configured to analyze kinematics.

144. The system of claim 126 wherein the network is configured to analyze the kinematics of an individual member of a team or group.

145. The system of claim 126 wherein the network is configured to analyze the kinematics of a composite of a plurality of team or group members.

146. The system of claim 126 wherein the auxiliary device is configured to be associated with an individual, and comprises one or more sensors configured to obtain information from the individual and to transmit the information or analysis derived therefrom directly or indirectly to a network.

147. The system of claim 146 wherein the network interfaces with a mobile device.

148. The system of claim 147 wherein the network interfacing with the mobile device provides sensor information or analysis to a user.

149. The system of claim 148 wherein the system is configured to give the user of the mobile device information feedback regarding a physiological characteristic of a current activity.

150. The system of claim 149 wherein the current activity is selected from the group consisting of running, jogging, walking, sleeping, and a physical characteristic of playing a sport.

151. The system of claim 126 comprising a network configured to analyze one or more performance parameters of a team sport or group activity.

152. The system of claim 126 comprising a network configured to analyze one or more performance parameters of horse racing.

153. The system of claim 126 comprising a network configured to provide an electronic medical records functionality.

154. A customizable tool kit or platform for building a diagnostic or other system to provide information, analysis or alerts for an animal, animals, human or humans, comprising a kit or platform of customizable components to meet the needs of a developer, consumer or user of the system, the components comprising at least one sensor insertable within or upon an oral cavity receptacle configured for placement in the oral cavity of the animal, animals, human or humans, at least one oral cavity receptacle configured to contain or receive the sensor, optionally, at least one smart auxiliary device which is wearable, attachable or insertable externally to the oral cavity, and at least one network unit configured to receive information, analysis or alerts from or transmit information, analysis or alerts to the at least one oral cavity sensor or the at least one auxiliary device, or both, and analyze, transmit, or both, the information, analysis or alerts obtained or received, wherein components for selecting the auxiliary devices, the oral cavity sensor receptacles, the sensors, and the network units are made available to the developer, consumer or user to construct or have constructed a system configured to obtain or transmit information, analysis or alerts customized to meet the specific needs of the developer, consumer or user.

155. The tool kit or platform of claim 154 wherein the tool kit or platform comprises at least one auxiliary device.

156. The tool kit or platform of claim 154 wherein a preselected set of kit or platform components is provided in the kit or platform together with instructions for building the desired system.

157. The tool kit or platform of claim 154 wherein the system is designed for a sports function, health analytics, diagnostic analytics, performance analytics; integration of body sensors, health-devices, nano-particles, sports and performance sensors on inanimate objects and sports equipment; customizable developers' tool kit for biosensors, sensors, performance, medical analytics, oral and systemic diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision and analytics.

158. The tool kit or platform of claim 154 wherein the animal is a pet.

159. The tool kit or platform of claim 158 wherein the pet is a dog or cat.

160. The tool kit or platform of claim 154 wherein the smart auxiliary device is a smart collar.

161. The tool kit or platform of claim 154 wherein the system further comprises a historic database of the animal or human as to one or more characteristics from which comparisons or analyses are to be made.

162. The tool kit or platform of claim 154 wherein the system further comprises a database of animals or humans having a common characteristic to the animal or human on which the auxiliary device is located and for which a predetermined comparison is to be made.

163. The tool kit or platform of claim 154 wherein the animal is a pet, the auxiliary device comprises a smart collar which connects via WiFi to an accessible portal and wherein the system provides information or analysis regarding the pet obtained from or transmitted to one or more sensors located within the oral cavity of the pet.

164. The tool kit or platform of claim 154 wherein the system comprises a database compilation of one or more of the pet's biological or physiological attributes.

165. The tool kit or platform of claim 154 wherein the system comprises a database compilation of one or more attributes of animals having at least one similar characteristic to the pet.

166. The tool kit or platform of claim 154 wherein the auxiliary device is associated with one or more team or group members of a team or group, comprises one or more sensors also associated with the one or more team or group members, and the auxiliary device is configured to transmit the information from the one or more sensors to a network.

167. The tool kit or platform of claim 154 wherein the oral device is a mouth guard and the one or more sensors measures oxygen levels or heart rate.

168. The tool kit or platform of claim 154 wherein the network analyzes fatigue.

169. The tool kit or platform of claim 168 wherein the network analyzes the fatigue of an individual team member.

170. The tool kit or platform of claim 168 wherein the network analyzes the fatigue of a composite of a plurality of team members.

171. The tool kit or platform of claim 154 wherein the network analyzes an aspect of a team or group performance criteria of a team or a group.

172. The tool kit or platform of claim 154 wherein the network analyzes one or more components of horse racing performance.

173. The tool kit or platform of claim 154 wherein the auxiliary device is configured to be associated with an individual and is configured to transmit information to a network.

174. The tool kit or platform of claim 173 wherein the network interfaces with a mobile device.

175. The tool kit or platform of claim 174 wherein the network interfacing with a mobile device provides sensor information to a user.

176. The tool kit or platform of claim 175 wherein the system is configured to give the user of the mobile device information feedback regarding a biological or physiological characteristic of a current activity.

177. The tool kit or platform of claim 176 wherein the current activity is selected from the group consisting of running, jogging, walking, sleeping, and a physical characteristic of playing a sport.

178. The tool kit or platform of claim 154 wherein the network provides an electronic medical records functionality.

179. A method for monitoring blood components of an animal or human comprising the steps of inserting a device and monitoring the device or monitoring an inserted device configured to be inserted into an oral cavity of an animal or human, wherein the device comprises a smart sensor receptacle for one or more sensors, wherein the smart receptacle contains or receives one or more sensors capable of providing or receiving information or analysis related to the animal or human's blood components, using the sensors to monitor or analyze one or more aspects of the blood components and transmitting information or analysis to, from or among a network or networks capable of utilizing the information or analysis.

180. The method of claim 179 wherein the device is configured to collect blood from bleeding due to gum disease, oral trauma and injury, testing, teeth or gum cleansing, flossing, water picking, brushing, pin-pricking, or other inducement of bleeding.

181. The method of claim 179 wherein the device is configured to be inserted into the oral cavity to be bathed in blood to measure blood glucose levels, blood composition, medication, blood chemistry, or other characteristics.

182. The method of claim 179 wherein the device is configured to collect the blood from the bleeding area to detect glucose, blood chemicals, medication, or other blood-related characteristics.

183. The method of claim 179 wherein the method further comprises data storage.
